(19) United States
(12) Patent Application Publication
(10) Pub. No.: US 2024/0190864 A1
(43) Pub. Date: Jun. 13, 2024

(54) Bruton's Tyrosine Kinase (BTK) INHIBITOR AND APPLICATION THEREOF

(71) Applicant: YA THERAPEUTICS INC, Shanghai (CN)

(72) Inventors: Yonghong LIANG, Shanghai (CN); Zhiyong XU, Shanghai (CN); Zhaosen ZENG, Shanghai (CN); Wenguang YAN, Shanghai (CN); Fangjun XIONG, Shanghai (CN)

(21) Appl. No.: 18/261,883

(22) PCT Filed: Dec. 27, 2021
(86) PCT No.: PCT/CN2021/141767

§ 371 Date: Jul. 18, 2023

(30) Foreign Application Priority Data
Feb. 3, 2021 (CN) 202110152506.4
Feb. 23, 2021 (CN) 202110203074.5
Feb. 23, 2021 (CN) 202110203701.5

(51) Int. Cl. C07D 471/04; A61K 31/4545; A61K 31/4985; A61K 45/06; A61P 35/00

(52) U.S. Cl. CPC C07D 471/04; A61K 31/4545; A61K 31/4985; A61K 45/06; A61P 35/00

(57) ABSTRACT

Disclosed are a heterocyclic compound acting as a BTK (Bruton's Tyrosine Kinase) inhibitor, a preparation method and a medical application thereof. Specifically, the present disclosure relates to a compound represented by the general formula (I) and a pharmaceutically acceptable salt thereof, a pharmaceutical composition containing the compound and/or the pharmaceutically acceptable salt thereof, and a use applying the compound or the pharmaceutically acceptable salt in the treatment or prevention of BTK-related disorders, especially tumors. The present disclosure relates to a class of heterocyclic compounds and at the same time provides the preparation method for the pharmaceutical composition containing this class of compounds or the pharmaceutically acceptable salt thereof. Each substituent of the general formula (I) has the same definition as that in the specification.

### CROSS-REFERENCE TO RELATED APPLICATION

This application claims priority to International Application Nos. PCT/CN2021101525064, filed on Feb. 4, 2021, International Application Nos. PCT/CN2021102030745, filed on Feb. 23, 2021, International Application Nos. PCT/CN2021102037015, filed on Feb. 24, 2021, and the content of each of which is incorporated herein by reference in its entirety.

### TECHNICAL FIELD

The present disclosure relates to a compound acting as a Bruton's Tyrosine Kinase (BTK) inhibitor, which is useful for treating diseases capable of being treated by inhibiting BTK. The present disclosure further provides a pharmaceutical composition containing the compound and a method for preparing the compound.

### BACKGROUND

BTK is Tec family non-receptor protein kinase. As the second largest family next only to a Src family in the human non-receptor kinase Tec mainly includes BTK, BMX (etk), ITK, TEC and TXK (PLK). BTK was determined as a defective protein in XLA (X-linked agammaglobulinemia) in 1993. This protein is all expressed in each development stage of the cell B (except for the finally differentiated plasmocyte), and during a process that the pre-B lymphocyte is transited to an anaphase cell B, BKT is a necessary gene for cell differentiation and cell proliferation, and expressed in all B-cell lymphoma, ALL (Acute Lymphocytic Leukemia) and plasmacytoma. In addition, BKT also has a few expressions in bone marrow cells and erythroid progenitor cells.

At present, BTK small-molecule inhibitors, including ibrutinib, acalabrutinib and Zanubrutinib, have been approved for marketing by American FDA, and are used for treating MCL ((Mantle Cell Lymphoma) and CLL (Chronic Lymphocytic Leukemia).

Although ibrutinib, acalabrutinib and Zanubrutinib have remarkable therapeutic effect, a considerable proportion of clinical B-cell lymphoma patients are insensitive to the treatment except that some patients have drug resistance in later stages, for example, about ⅓ of MCL patients have no response to the treatment, and the response rate in DLBCL (Diffuse Large B-cell Lymphoma) is also not high. In view of the above problem, the BTK inhibitor with high activity and strong specificity still needs to be developed in this field.

### SUMMARY

In order to solve the problem, the present disclosure provides a compound or a stereoisomer thereof, a stable isotope derivative, a hydrate, a solvate and a pharmaceutically acceptable salt for a novel BTK (Bruton's Tyrosine Kinase) represented by the formula (I):

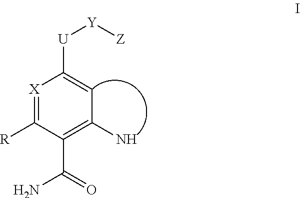

R may be independently selected from H, D,

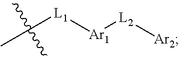

L₁ is independently selected from —C₀₋₄alkyl-, —CR¹R²—, —C₁₋₂alkyl(R¹)(OH)—, —C(O)—, —CR¹R²O—, —OCR¹R²—, —SCR¹R²—, —CR¹R²S—, —NR¹—, —NR¹C(O)—, —C(O)NR¹—, —NR¹C(O)NR²—, —CF₂—, —O—, —S—, —S(O)ₘ—, —NR¹S(O)₂—, —S(O)₂NR¹—;
L₂ is independently selected from —C₀₋₄alkyl-, —CR¹R²—, —C₁₋₂alkyl(R¹)(OH)—, —C(O)—, —CR¹R²O—, —OCR¹R²—, —SCR¹R²—, —CR¹R²S—, —NR¹—, —NR¹C(O)—, —C(O)NR¹—, —NR¹C(O)NR₂—, —CF₂—, —O—, —S—, —S(O)ₘ—, —NR¹S(O)₂—, —S(O)₂NR¹—;
Ar₁ and Ar₂ are independently selected from phenyl, 5-6 membered heteroaryl, and the phenyl and 5-6 membered heteroaryl are optionally substituted with one or more G¹;
X may be independently selected from N, CR³;

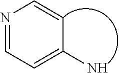

is independently selected from the structure below:

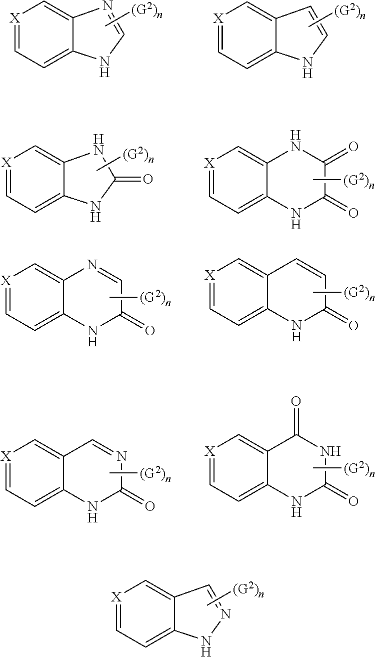

n is 1 or 2;
U is independently selected from bond, —C₁₋₄alkyl-, —CR⁴R⁵—, —C₁₋₂alkyl(R⁴)(OH)—, —C(O)—, —CR⁴R⁵O—, —OCR⁴R⁵—, —SCR⁴R⁵—, —CR⁴R⁵S—, —NR⁴—, —NR⁴C(O)—, —C(O)NR⁴—, —NR⁴C(O)NR⁵—, —CF₂—, —O—, —S—, —S(O)ₘ—, —NR⁴S(O)₂—, —S(O)₂NR⁴—;
Y is absent or selected from C₃₋₈cycloalkyl, 3-8 membered heterocycloalkyl, 5-12 membered bicycloalkyl, 5-12 membered heterobicycloalkyl, 5-12 membered spirocycloalkyl, 5-12 membered heterospirocycloalkyl, aryl or heteroaryl, and the C₃₋₈ cycloalkyl, 3-8 membered heterocycloalkyl, 5-12 membered bicycloalkyl, 5-12 membered heterobicycloalkyl, 5-12 membered spirocycloalkyl, 5-12 membered heterospirocycloalkyl, aryl or heteroaryl is optionally substituted with one or more G³;
Z is independently selected from CN, —NR¹²CN,

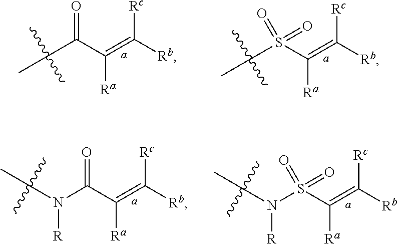

Bond a is a double bond or a triple bond;
When a is a double bond, R^{a}, R^{b}, and R^{c} are each independently selected from H, D, —CN, halogen, C₁₋₆alkyl, C₃₋₆cycloalkyl or 3-6₆heterocycloalkyl, and the C₁₋₆alkyl, C₃₋₆cycloalkyl and 3-6₆heterocycloalkyl are optionally substituted with one or more G⁴;
When a is a triple bond, R^{a} and R^{c} are absent, R^{b} is independently selected from H, D and —CN, and the halogen, C₁₋₆alkyl, C₃₋₆cycloalkyl or 3-6 heterocycloalkyl is optionally substituted with one or more G⁵;
R¹² is independently selected from H, D, C₁₋₆alkyl, C₃₋₆cycloalkyl or 3-6₆heterocycloalkyl, and the C₁₋₆alkyl, C₃₋₆cycloalkyl and 3-6 heterocycloalkyl are optionally substituted with one or more G⁶;
G¹, G², G³, G⁴, G⁵ and G⁶ are each independently selected from H, D, —CN, halogen, C₁₋₆alkyl, C₂₋₆alkenyl, C₂₋₆alkynyl, C₃₋₈cycloalkyl or 3-8 heterocycloalkyl, C₆₋₁₀ membered aryl, 5-10 membered heteroaryl, —OR⁶, —OC(O)NR⁶R⁷, —C(O)OR⁶, —C(O)NR⁶R⁷, —C(O)R⁶, —NR⁶R⁷, —NR⁶C(O)R⁷, —NR⁶C(O)NR⁷R⁸, —S(O)ₘR⁶ or —NR⁶S(O)ₘR⁷, and the C₁₋₆alkyl, C₂₋₆alkenyl, C₂₋₆alkynyl, C₃₋₈cycloalkyl, 3-8 heterocycloalkyl, C₆₋₁₀ membered aryl, 5-10 membered heteroaryl are optionally substituted with one or more —CH, halogen, C₁₋₆alkyl, C₂₋₆alkenyl, C₂₋₆alkynyl, C₃₋₈cycloalkyl or 3-8 heterocycloalkyl, C₆₋₁₀ membered aryl, 5-10 membered heteroaryl, —OC(O)NR⁹R¹⁰, —C(O)OR⁹, —C(O)NR⁹R¹⁰, —C(O)R⁹, —NR⁹R¹⁰, —NR⁹C(O)R¹⁰, —NR⁹C(O)NR¹⁰R¹¹, —S(O)ₘR⁹ or —NR⁹S(O)ᵢR¹⁰;
R³, R⁴, R⁵, R⁶, R⁷, R⁸, R⁹, R¹⁰ and R¹¹ are each independently selected from H, D, —CN, halogen, C₁₋₆alkyl-, C₃₋₈cycloalkyl or 3-8 heterocycloalkyl, aryl or heteroaryl; and
m is 1 or 2.

In some implementation modes, according to the compound represented by the general formula (I), the pharmaceutically acceptable salt or the stereoisomer thereof, the general formula (I) is further represented by the general formula Ia:

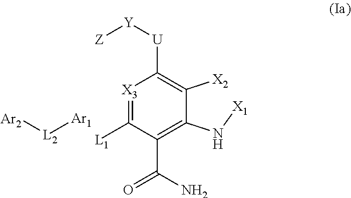

X₁, X₂ and X₃ may be independently selected from N, CR¹;
Ar₁ and Ar₂ are independently selected from phenyl, 5-6 membered heteroaryl, and the phenyl and 5-6 membered heteroaryl are optionally substituted with one or more G¹;
R¹ is independently selected from H, D, —CN, halogen, C₁₋₆alkyl, COOH, CONH₂, NHCOH, CONHR², OR² or —NHR²;
R² is independently selected from H, D, —CN, halogen, C₁₋₆alkyl, C₃₋₆cycloalkyl, 3-6 heterocycloalkyl, —OR³, —NR³R⁴ and —C(O)NR³R⁴, and the C₁₋₆alkyl, C₃₋₆cycloalkyl or 3-6 heterocycloalkyl is optionally substituted with —CN, halogen, —OR⁵, —NR⁵R⁶, C₁₋₆alkyl, C₃₋₆cycloalkyl or 3-6 heterocycloalkyl;
L₁, L₂ and U are independently selected from bond, —C₁₋₄alkyl-, —CR⁷R⁸—, —C₁₋₂alkyl(R⁷)(OH)—, —C(O)—, —CR⁷R⁸O—, —OCR⁷R⁸—, —SCR⁷R⁸—, —CR⁷R⁸S—, —NR⁷C(O)—, —C(O)NR⁷—, —NR⁷C(O)NR⁸—, —CF₂—, —O—, —S—, —S(O)ₘ—, —NR⁷S(O)₂—, —S(O)₂NR⁸—;
Y is absent or selected from C₃₋₈cycloalkyl, 3-8 membered heterocycloalkyl, 5-12 membered bicycloalkyl, 5-12 membered heterobicycloalkyl, 5-12 membered spirocycloalkyl, 5-12 membered heterospirocycloalkyl, aryl or heteroaryl, and the C₃₋₈cycloalkyl, 3-8 membered heterocycloalkyl, 5-12 membered bicycloalkyl, 5-12 membered heterobicycloalkyl, 5-12 membered spirocycloalkyl, 5-12 membered heterospirocycloalkyl, aryl or heteroaryl is optionally substituted with one or more G²;
Z is independently selected from CN, —NR⁹CN,

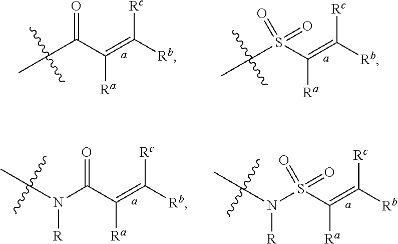

Bond a is a double bond or a triple bond;
When a is a double bond, R^{a}, R^{b} and R^{c} are independently selected from H, D, —CN, halogen, C₁₋₆alkyl, C₃₋₆cycloalkyl or 3-6 heterocycloalkyl, and the C₁₋₆alkyl, C₃₋₆cycloalkyl and 3-6 heterocycloalkyl are optionally substituted with one or more G³;
R^{a} and R^{b} or R^{b} and R^{c} optionally form a 3-6 membered ring containing heteroatoms with their connected carbon atoms;
When a is a triple bond, R^{a} and R^{c} are absent, each of R^{b} is independently selected from H, D, and —CN, and the halogen, C₁₋₆alkyl, C₃₋₆cycloalkyl or 3-6 heterocycloalkyl is optionally substituted with one or more G⁴;
R¹² is independently selected from H, D, C₁₋₆alkyl, C₃₋₆cycloalkyl or 3-6 heterocycloalkyl, and the C₁₋₆alkyl, C₃₋₆cycloalkyl and 3-6 heterocycloalkyl are optionally substituted with one or more G⁵;
G¹, G², G³, G⁴ and G⁵ are each independently selected from —CN, halogen, C₁₋₆alkyl, C₂₋₆alkenyl, C₂₋₆alkynyl, C₃₋₈cycloalkyl or 3-8 heterocycloalkyl, C₆₋₁₀ membered aryl, 5-10 membered heteroaryl, —OR¹⁰, —OC(O)NR¹⁰R¹¹, —C(O)OR¹⁰, —C(O)NR¹⁰R¹¹, —C(O)R¹⁰, —NR¹⁰R¹¹, —NR¹⁰C(O)R¹¹, —NR¹⁰C(O)NR¹¹R¹², —S(O)ₘR¹⁰ or —NR¹⁰S(O)ₘR¹¹, and the C₁₋₆alkyl, C₂₋₆alkenyl, C₂₋₆alkynyl, C₃₋₈cycloalkyl or 3-8 heterocycloalkyl, C₆₋₁₀ membered aryl, 5-10 membered heteroaryl are optionally substituted with the substituents of one or more —CN, halogen, C₁₋₆alkyl, C₂₋₆alkenyl, C₂₋₆alkynyl, C₃₋₈cycloalkyl or 3-8 heterocycloalkyl, C₆₋₁₀ membered aryl, 5-10 membered heteroaryl, —OR¹³, —OC(O)NR¹³R¹⁴, —C(O)OR¹³, —C(O)NR¹³R¹⁴, —C(O)R¹³, —NR¹³R¹⁴, —NR¹³C(O)R¹⁴, —NR¹³C(O)NR¹³R¹⁵, —S(O)ₘR¹³ or —NR¹³S(O)ᵢR¹⁴;
R³, R⁴, R⁵, R⁶, R⁷, R⁸, R⁹, R¹⁰, R¹¹, R¹³, R¹⁴ and R¹⁵ are each independently selected from H, —CN, halogen, C₁₋₆alkyl-, C₃₋₈cycloalkyl or 3-8 heterocycloalkyl, aryl or heteroaryl; and
m is 1 or 2.

In some implementation modes, according to the compound represented by the general formula (I), the pharmaceutically acceptable salt or the stereoisomer thereof, the general formula (I) is further represented by the general formula Ib:

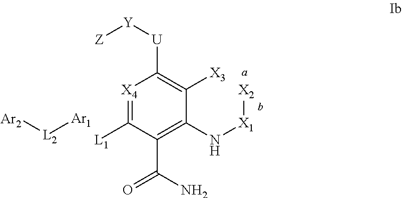

X₁, X₂, X₃ and X₄ are selected from N, CR¹;
Bonds a and b are single or double bonds;
Ar₁ and Ar₂ are independently selected from phenyl or 5-6 membered heteroaryl, and the phenyl and 5-6 membered heteroaryl are optionally substituted with one or more G¹;
R¹ is independently selected from H, D, —CN, halogen, C₁₋₆alkyl, COOH, CONH₂, NHCOH, CONHR², OR² or —NHR²;
R² is independently selected from H, D, —CN, halogen, C₁₋₆alkyl, C₃₋₆cycloalkyl, 3-6 heterocycloalkyl, —OR³, —NR³R⁴ and —C(O)NR³R⁴, and the C₁₋₆alkyl, C₃₋₆cycloalkyl or 3-6 heterocycloalkyl is optionally substituted with —CN, halogen, —OR⁵, —NR⁵R⁶, C₁₋₆alkyl, C₃₋₆cycloalkyl or 3-6 heterocycloalkyl;
L₁, L₂ and U are independently selected from —C₁₋₄alkyl-, —CR⁷R⁸—, —C₁₋₂alkyl(R⁷)(OH)—, —C(O)—, —CR⁷R⁸O—, —OCR⁷R⁸—, —SCR⁷R⁸—, —CR⁷R⁸S—, —NR⁷C(O)—, —C(O)NR⁷—, —NR⁷C(O)NR⁸—, —CF₂—, —O—, —S—, —S(O)ₘ—, —NR⁷S(O)₂—, —S(O)₂NR⁸—;
Y is absent or selected from C₃₋₈cycloalkyl, 3-8 membered heterocycloalkyl, 5-12 membered bicycloalkyl, 5-12 membered heterobicycloalkyl, 5-12 membered spirocycloalkyl, 5-12 membered heterospirocycloalkyl, aryl or heteroaryl, and the C₃₋₈cycloalkyl, 3-8 membered heterocycloalkyl, 5-12 membered bicycloalkyl, 5-12 membered heterobicycloalkyl, 5-12 membered spirocycloalkyl, 5-12 membered heterospirocycloalkyl, aryl or heteroaryl is optionally substituted with one or more G²;
Z is independently selected from CN, —NR¹²CN,

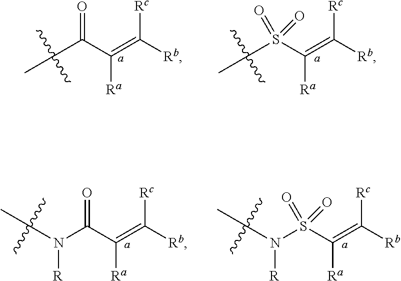

Bond c is a double bond or a triple bond;
When c is a double bond, R^{a}, R^{b}, and R^{c} are independently selected from H, D, —CN, halogen, C₁₋₆alkyl, C₃₋₆cycloalkyl or 3-6 heterocycloalkyl, and the C₁₋₆alkyl, C₃₋₆cycloalkyl and 3-6 heterocycloalkyl are optionally substituted with one or more G³;
R^{a} and R^{b} or R^{b} and R^{c} optionally form a 3-6 membered ring containing heteroatoms with their connected carbon atoms;
When c is a triple bond, R^{a} and R^{c} are absent, R^{b} is independently selected from H, D and —CN, and the halogen, C₁₋₆alkyl, C₃₋₆cycloalkyl or 3-6 heterocycloalkyl is optionally substituted with one or more G⁴;
R⁹ is independently selected from H, D, C₁₋₆alkyl, C₃₋₆cycloalkyl or 3-6 heterocycloalkyl, and the C₁₋₆alkyl, C₃₋₆cycloalkyl and 3-6 heterocycloalkyl are optionally substituted with one or more G⁵;
G¹, G², G³, G⁴ and G⁵ are each independently selected from —CN, halogen, C₁₋₆alkyl, C₂₋₆alkenyl, C₂₋₆alkynyl, C₃₋₈cycloalkyl or 3-8 heterocycloalkyl, C₆₋₁₀ membered aryl, 5-10 membered heteroaryl, —OR¹⁰, —OC(O)NR¹⁰R¹¹, —C(O)OR¹⁰, —C(O)NR¹⁰R¹¹, —C(O)R¹⁰, —NR¹⁰R¹¹, —NR¹⁰C(O)R¹¹, —NR¹⁰C(O)NR¹¹R¹², —S(O)ₘR¹⁰ or —NR¹⁰S(O)ₘR¹¹, and the C₁₋₆alkyl, C₂₋₆alkenyl, C₂₋₆alkynyl, C₃₋₈cycloalkyl or 3-8 heterocycloalkyl, C₆₋₁₀ membered aryl and 5-10 membered heteroaryl are optionally substituted with the substituents of one or more —CN, halogen, C₁₋₆alkyl, C₂₋₆alkenyl, C₂₋₆alkynyl, C₃₋₈cycloalkyl or 3-8 heterocycloalkyl, C₆₋₁₀ membered aryl, 5-10 membered heteroaryl, —OR¹³, —OC(O)NR¹³R¹⁴, —C(O)OR¹³, —C(O)NR¹³R¹⁴, —C(O)R¹³, —NR¹³R¹³, —NR¹³C(O)R¹⁴, —NR¹³C(O)NR¹⁴R¹⁵, —S(O)ₘR¹³ or —NR¹³S(O)ₘR¹⁴;
R³, R⁴, R⁵, R⁶, R⁷, R⁸, R⁹, R¹⁰, R¹¹, R¹³, R¹⁴ and R¹⁵ are each independently selected from H, —CN, halogen, C₁₋₆alkyl-, C₃₋₈cycloalkyl or 3-8 heterocycloalkyl, aryl or heteroaryl; and
m is 1 or 2.

In some implementation modes, according to the compound represented by the general formula (I), the pharmaceutically acceptable salt or the stereoisomer thereof, the general formula (I) is further represented by the general formula Ic:

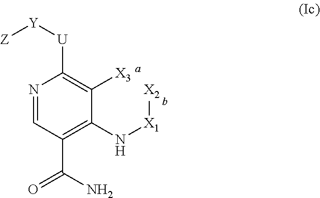

X₁, X₂ may be independently selected from N, CR¹;
X₃ may be absent or independently selected from N, CR¹;
Bonds a and b are single or double bonds;
R¹ is independently selected from H, D, —CN, halogen, C₁₋₆alkyl, COOH, CONH₂, NHCOH, CONHR², OR² or —NHR²;
R² is independently selected from H, D, —CN, halogen, C₁₋₆alkyl, C₃₋₆cycloalkyl, 3-6 heterocycloalkyl, —OR³, —NR³R⁴ and —C(O)NR³R⁴, and C₁₋₆alkyl, C₃₋₆cycloalkyl and 3-6 heterocycloalkyl is optionally substituted with —CN, halogen, —OR⁵, —NR⁵R⁶, C₁₋₆alkyl, C₃₋₆cycloalkyl, or 3-6 heterocycloalkyl;
U is independently selected from bond, —C₁₋₄alkyl-, —CR⁷R⁸—, —C₁₋₂alkyl(R⁷)(OH)—, —C(O)—, —CR⁷R⁸O—, —OCR⁷R⁸—, —SCR⁷R⁸—, —CR⁷R⁸S—, —NR⁷C(O)—, —C(O)NR⁷—, —NR⁷C(O)NR⁸—, —CF₂—, —O—, —S—, —S(O)ₘ—, —NR⁷S(O)₂—, —S(O)₂NR⁷—;
Y is absent or selected from C₃₋₈cycloalkyl, 3-8 membered heterocycloalkyl, 5-12 membered bicycloalkyl, 5-12 membered heterobicycloalkyl, 5-12 membered spirocycloalkyl, 5-12 membered heterospirocycloalkyl, aryl or heteroaryl, and the C₃₋₈cycloalkyl, 3-8 membered heterocycloalkyl, 5-12 membered bicycloalkyl, 5-12 membered heterobicycloalkyl, 5-12 membered spirocycloalkyl, 5-12 membered heterospirocycloalkyl, aryl or heteroaryl is optionally substituted with one or more G¹;
Z is independently selected from CN, —NR¹²CN,

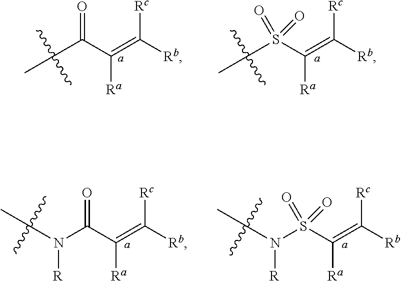

Bond c is a double bond or a triple bond;
When c is a double bond, R^{a}, R^{b}, and R^{c} are each independently selected from H, —CN, halogen, C₁₋₆alkyl, C₃₋₆cycloalkyl or 3-6 heterocycloalkyl, and the C₁₋₆alkyl, C₃₋₆cycloalkyl and 3-6 heterocycloalkyl are optionally substituted with one or more G²;
R^{a} and R^{b} or R^{b} and R^{c} optionally form a 3-6 membered ring containing heteroatoms with their connected carbon atoms;
When c is a triple bond, R^{a} and R^{c} are absent, R^{b} is independently selected from H and —CN, and the halogen, C₁₋₆alkyl, C₃₋₆cycloalkyl or 3-6 heterocycloalkyl is optionally substituted with one or more G³;
R⁹ is independently selected from H, C₁₋₆alkyl, C₃₋₆cycloalkyl or 3-6 heterocycloalkyl, and the C₁₋₆alkyl, C₃₋₆cycloalkyl and 3-6 heterocycloalkyl are optionally substituted with G⁴;
G¹, G², G³ and G⁴ are each independently selected from —CN, halogen, C₁₋₆alkyl, C₂₋₆alkenyl, C₂₋₆alkynyl, C₃₋₈cycloalkyl or 3-8 heterocycloalkyl, C₆₋₁₀ membered aryl, 5-10 membered heteroaryl, —OR¹⁰, —OC(O)NR¹⁰R¹¹, —C(O)OR¹⁰, —C(O)NR¹⁰R¹¹, —C(O)R¹⁰, —NR¹⁰R¹¹, —NR¹⁰C(O)R¹¹, —NR¹⁰C(O)NR¹¹R¹², —S(O)ₘR¹⁰ or —NR¹⁰S(O)ₘR¹¹, and the C₁₋₆alkyl, C₂₋₆alkenyl, C₂₋₆alkynyl, C₃₋₈cycloalkyl or 3-8 heterocycloalkyl, C₆₋₁₀ membered aryl, 5-10 membered heteroaryl are optionally substituted with the substituents of one or more —CN, halogen, C₁₋₆alkyl, C₂₋₆alkenyl, C₂₋₆alkynyl, C₃₋₈cycloalkyl or 3-8 heterocycloalkyl, C₆₋₁₀ membered aryl, 5-10 membered heteroaryl, —OR¹³, —OC(O)NR¹³R¹⁴, —C(O)OR¹³, —C(O)NR¹³R¹⁰, —C(O)R¹³, —NR¹³R¹⁴, —NR¹³C(O)R¹⁴, —NR¹³C(O)NR¹³R¹⁵, —S(O)ₘR¹³ or —NR¹³S(O)ᵢR¹⁴;
R³, R⁴, R₅, R⁶, R⁷, R⁸, R⁹, R¹⁰, R¹¹, R¹³, R¹⁴ and R¹⁵ are each independently selected from H, —CN, halogen, C₁₋₆alkyl-, C₃₋₈cycloalkyl or 3-8 heterocycloalkyl, aryl or heteroaryl; and
m is 1 or 2.

In some implementation modes, the compound represented by the general formula (I), or the stereoisomer, the solvate or the prodrug thereof, or the pharmaceutically acceptable salts thereof are selected from the following compound, the stereoisomer, the solvate or the prodrug thereof, or the pharmaceutically acceptable salts thereof:

| | |
|---|---|
| 1 | (S)-4-(3-(but-2-ynamido)piperidin-1-yl)-2-oxo-6-(4-phenoxyphenyl)-2,3-dihydro-1H- |
| | imidazo[4,5-c]pyridine-7-carboxamide |
| 2 | (S)-4-(3-(but-2-ynamido)piperidin-1-yl)-6-(4-phenoxyphenyl)-1H-pyrazolo[4,3-c]pyridine- |
| | 7-carboxamide |
| 3 | (S)-4-(3-acrylamidopiperidin-1-yl)-2-oxo-6-(4-phenoxyphenyl)-2,3-dihydro-1H-imidazo[4,5- |
| | c]pyridine-7-carboxamide |
| 4 | (S)-4-(3-acrylamidopyrrolidin-1-yl)-2-oxo-6-(4-phenoxyphenyl)-2,3-dihydro-1H-imidazo[4,5- |
| | c]pyridine-7-carboxamide |
| 5 | (S)-4-(3-(but-2-ynamido)pyrrolidin-1-yl)-2-oxo-6-(4-phenoxyphenyl)-2,3-dihydro-1H- |
| | imidazo[4,5-c]pyridine-7-carboxamide |
| 6 | (R)-4-(3-(but-2-ynamido)piperidin-1-yl)-2-oxo-6-(4-phenoxyphenyl)-2,3-dihydro-1H- |
| | imidazo[4,5-c]pyridine-7-carboxamide |
| 7 | (R)-4-(3-(but-2-ynamido)piperidin-1-yl)-6-(4-phenoxyphenyl)-1H-pyrazolo[4,3-c]pyridine- |
| | 7-carboxamide |
| 8 | (R)-4-(3-acrylamidopiperidin-1-yl)-2-oxo-6-(4-phenoxyphenyl)-2,3-dihydro-1H-imidazo[4,5- |
| | c]pyridine-7-carboxamide |
| 9 | (R)-4-(3-(but-2-ynamido)pyrrolidin-1-yl)-2-oxo-6-(4-phenoxyphenyl)-2,3-dihydro-1H- |
| | imidazo[4,5-c]pyridine-7-carboxamide |
| 10 | (S)-5-(3-(but-2-ynamido)piperidin-1-yl)-2,3-dioxo-7-(4-phenoxyphenyl)-1,2,3,4- |
| | tetrahydropyrido[3,4-b]pyrazine-8-carboxamide |
| 11 | (R)-5-(3-(but-2-ynamido)piperidin-1-yl)-2,3-dioxo-7-(4-phenoxyphenyl)-1,2,3,4- |
| | tetrahydropyrido[3,4-b]pyrazine-8-carboxamide |
| 12 | (S)-5-(3-acrylamidopiperidin-1-yl)-2,3-dioxo-7-(4-phenoxyphenyl)-1,2,3,4- |
| | tetrahydropyrido[3,4-b]pyrazine-8-carboxamide |
| 13 | (R)-5-(3-acrylamidopiperidin-1-yl)-2,3-dioxo-7-(4-phenoxyphenyl)-1,2,3,4- |
| | tetrahydropyrido[3,4-b]pyrazine-8-carboxamide |
| 14 | (S)-5-(3-(but-2-ynamido)pyrrolidin-1-yl)-2,3-dioxo-7-(4-phenoxyphenyl)-1,2,3,4- |
| | tetrahydropyrido[3,4-b]pyrazine-8-carboxamide |
| 15 | (R)-5-(3-(but-2-ynamido)pyrrolidin-1-yl)-2,3-dioxo-7-(4-phenoxyphenyl)-1,2,3,4- |
| | tetrahydropyrido[3,4-b]pyrazine-8-carboxamide |
| 16 | (S)-5-(3-acrylamidopyrrolidin-1-yl)-2,3-dioxo-7-(4-phenoxyphenyl)-1,2,3,4- |
| | tetrahydropyrido[3,4-b]pyrazine-8-carboxamide |
| 17 | (R)-5-(3-acrylamidopyrrolidin-1-yl)-2,3-dioxo-7-(4-phenoxyphenyl)-1,2,3,4- |
| | tetrahydropyrido[3,4-b]pyrazine-8-carboxamide |
| 18 | (S)-4-(3-(but-2-ynamido)piperidin-1-yl)-6-(4-phenoxyphenyl)-1H-imidazo[4,5-c]pyridine- |
| | 7-carboxamide |
| 19 | (S)-4-(3-(but-2-ynamido)piperidin-1-yl)-2-methyl-6-(4-phenoxyphenyl)-1H-imidazo[4,5- |
| | c]pyridine-7-carboxamide |
| 20 | 4-(1-acryloylpiperidin-4-yl)-2-oxo-6-(4-phenoxyphenyl)-2,3-dihydro-1H-imidazo[4,5- |
| | c]pyridine-7-carboxamide |
| 21 | (S)-4-(1-acryloylpiperidin-3-yl)-2-oxo-6-(4-phenoxyphenyl)-2,3-dihydro-1H-imidazo[4,5- |
| | c]pyridine-7-carboxamide |
| 22 | (S)-4-(1-acryloylpiperidin-3-yl)-2-oxo-6-(4-phenoxyphenyl)-2,3-dihydro-1H-imidazo[4,5- |
| | c]pyridine-7-carboxamide |
| 23 | (S)-4-(3-(but-2-ynamido)piperidin-1-yl)-6-(4-phenoxyphenyl)-1H-pyrrolo[3,2-c]pyridine- |
| | 7-carboxamide |
| 24 | (R)-4-(3-(but-2-ynamido)piperidin-1-yl)-6-(4-phenoxyphenyl)-1H-pyrrolo[3,2-c]pyridine- |
| | 7-carboxamide |
| 25 | 4-(1-acryloylpiperidin-4-yl)-6-(4-phenoxyphenyl)-1H-pyrrolo[3,2-c]pyridine-7-carboxamide |
| 26 | (S)-4-(1-acryloylpiperidin-3-yl)-6-(4-phenoxyphenyl)-1H-pyrrolo[3,2-c]pyridine-7- |
| | carboxamide |
| 27 | (R)-4-(1-acryloylpiperidin-3-yl)-6-(4-phenoxyphenyl)-1H-pyrrolo[3,2-c]pyridine-7- |
| | carboxamide |
| 28 | (S)-5-(3-(but-2-ynamido)piperidin-1-yl)-2,3-dioxo-1,2,3,4-tetrahydropyrido[3,4-b]pyrazine- |
| | 8-carboxamide |
| 29 | (R)-5-(3-(but-2-ynamido)piperidin-1-yl)-2,3-dioxo-1,2,3,4-tetrahydropyrido[3,4-b]pyrazine- |
| | 8-carboxamide |
| 30 | (S)-5-(3-acrylamidopiperidin-1-yl)-2,3-dioxo-1,2,3,4-tetrahydropyrido[3,4-b]pyrazine-8- |
| | carboxamide |
| 31 | (R)-5-(3-acrylamidopiperidin-1-yl)-2,3-dioxo-1,2,3,4-tetrahydropyrido[3,4-b]pyrazine-8- |
| | carboxamide |
| 32 | (S)-5-(3-(but-2-ynamido)pyrrolidin-1-yl)-2,3-dioxo-1,2,3,4-tetrahydropyrido[3,4-b]pyrazine- |
| | 8-carboxamide |
| 33 | (R)-5-(3-(but-2-ynamido)pyrrolidin-1-yl)-2,3-dioxo-1,2,3,4-tetrahydropyrido[3,4-b]pyrazine- |
| | 8-carboxamide |
| 34 | (S)-5-(3-acrylamidopyrrolidin-1-yl)-2,3-dioxo-1,2,3,4-tetrahydropyrido[3,4-b]pyrazine-8- |
| | carboxamide |
| 35 | (R)-5-(3-acrylamidopyrrolidin-1-yl)-2,3-dioxo-1,2,3,4-tetrahydropyrido[3,4-b]pyrazine-8- |
| | carboxamide |
| 36 | (S)-4-(3-(but-2-ynamido)piperidin-1-yl)-1H-imidazo[4,5-c]pyridine-7-carboxamide |
| 37 | (S)-4-(3-(but-2-ynamido)piperidin-1-yl)-2-methyl-1H-imidazo[4,5-c]pyridine-7-carboxamide |
| 38 | (S)-4-(3-(but-2-ynamido)piperidin-1-yl)-2-oxo-2,3-dihydro-1H-imidazo[4,5-c]pyridine-7- |
| | carboxamide |
| 39 | (R)-4-(3-(but-2-ynamido)piperidin-1-yl)-2-oxo-2,3-dihydro-1H-imidazo[4,5-c]pyridine-7- |
| | carboxamide |
| 40 | 4-(1-acryloylpiperidin-4-yl)-2-oxo-2,3-dihydro-1H-imidazo[4,5-c]pyridine-7-carboxamide |
| 41 | (S)-4-(1-acryloylamino |
| | piperidine-3-yl)-2--methyl-1H-imidazo[4,5-c]pyridine-7-carboxamide |
| 42 | (R)-4-(1-acryloylamino |
| | piperidine-3-yl)-2--methyl-1H-imidazo[4,5-c]pyridine-7-carboxamide |

The present disclosure provides a novel BTK inhibitor or a stereoisomer, a hydrate, a solvate, a polymorph, a pharmaceutically acceptable salt, and a use of a pharmaceutically acceptable carrier in preparing the BTK inhibitor.

Optionally, the pharmaceutical composition of this application also includes one or more medicinal excipients.

The medicinal excipients in this application refer to the excipients and additives used in the production and formulation of drugs. They refer to substances that have been reasonably evaluated in terms of safety and included in the drug formulation, except for active ingredients. In addition to shaping, serving as a carrier, and improving stability, medicinal excipients also have important functions such as solubilization, solubilization assistance, and controlled release. They are important components that may affect the quality, safety, and effectiveness of drugs. Medicinal excipients can be divided into natural, semisynthesis and total synthesis according to their sources; according to its function and use, it can be divided into: solvent, propellant, solubilizer, cosolvent, emulsifier, colorant, adhesive, disintegrant, filler, lubricant, wetting agent, osmotic pressure regulator, stabilizer, flow aid, taste correction agent, preservative, suspending agent, coating material, fragrance, anti adhesion agent, antioxidant, chelating agent, osmotic accelerator, pH regulator, buffer, plasticizer, surfactant, foaming agent, defoamer, thickener, encapsulating agent, moisturizing agent, absorbent, diluent, flocculant and anti flocculant, filter aid, release blocker, etc.; and according to its route of administration, it can be divided into oral administration, injection, mucous membrane, percutaneous or local administration, nasal or oral inhalation administration and eye administration. The same medicinal excipient can be used in medicinal preparations with different routes of administration, and has different functions and uses.

The pharmaceutical composition of this application can be made into various suitable dosage forms according to the route of administration.

When administered orally, the pharmaceutical composition can be made into any orally acceptable form, including but not limited to tablets, capsules, granules, pills, syrups, oral solutions, oral suspensions, and oral emulsions. Among them, the carrier used for tablets generally includes lactose and corn starch, and lubricants such as magnesium stearate can also be added. Diluents used in capsules generally include lactose and dried corn starch. Oral suspensions are typically used by mixing active ingredients with suitable emulsifiers and suspensions. Optionally, some sweeteners, fragrances, or colorants can be added to the above oral formulations.

When applied transdermally or locally, the pharmaceutical composition can be made into appropriate ointments, lotions, or liniments, in which the active ingredient is suspended or dissolved in one or more carriers. The carriers that can be used for ointment preparation include but are not limited to: mineral oil, liquid vaseline, white vaseline, propylene glycol, polyethylene oxide, propylene oxide, emulsified wax and water; carriers that can be used for lotions or liniments include but are not limited to: mineral oil, dehydrated sorbitol monostearate, Tween 60, hexadecane ester wax, hexadecane aryl alcohol, 2-octyldodecanol, benzyl alcohol and water.

The pharmaceutical composition can also be administered in the form of injection, including injection, sterile powder for injection, and concentrated solution for injection. Among them, the available carriers and solvents include water, Ringer's solution, and isotonic sodium chloride solution. In addition, sterilized non-volatile oils can also be used as solvents or suspension media, such as monoglycerides or diglycerides.

Optionally, the compound of the application, its pharmaceutically acceptable salt, its stereoisomer or its prodrug can be used in combination with the second therapeutic agent. Therefore, optionally, the pharmaceutical composition of this application also includes one or more second therapeutic agents. In some embodiments, the second therapeutic agent is a chemotherapy drug, a targeted anticancer drug, or an immunotherapy drug. In some embodiments, the second therapeutic agent is selected from rituximab, lenalidomide, fludarabine, Cyclophosphamide, doxorubicin, Vincristine, and prednisone.

One aspect of this application relates to the use of the compound of the application, its pharmaceutically acceptable salt, its stereoisomer or its prodrug in preparing drugs, and the drugs are used for preventing and/or treating the diseases and/or symptoms related to the excessive activity of Bruton Tyrosine kinase in the subject.

One aspect of this application relates to the use of the compound of the application, its pharmaceutically acceptable salt, its stereoisomer or its prodrug in preparing drugs, and the drugs are used for preventing and/or treating the diseases and/or symptoms related to the excessive activity of Bruton Tyrosine kinase in the subject.

In some embodiments, the disease and/or symptom related to the excessive activity of Bruton Tyrosine kinase is selected from tumors (such as blood tumors or solid tumors), inflammation or autoimmune diseases.

In certain embodiments, the blood tumor is selected from lymphoma, myeloma, lymphocytic leukemia, and acute myeloid leukemia.

In some embodiments, the solid tumor is selected from lung cancer, breast cancer, prostate cancer, stomach cancer, liver cancer, pancreatic cancer, ovarian cancer, and colon cancer.

In some embodiments, the inflammatory or autoimmune disease is selected from rheumatoid arthritis, lupus erythematosus, Lupus nephritis, Multiple sclerosis, Schogren's syndrome, and underlying disease asthma.

In some embodiments, the subject is a mammal; for example, bovine, equine, ovine, swine, canine, feline and, rodent; and a primate; for example, human.

Another aspect of this application relates to the use of the compound of this application, its pharmaceutically acceptable salt, its stereoisomer or its prodrug in preparing preparations, which are used for reducing or inhibiting the activity of Bruton Tyrosine kinase in cells.

In some embodiments, the preparation is administered to the subject (such as the mammal; including bovine, equine, ovine, swine, canine, feline and rodent; and the primate; such as human) to reduce or inhibit the activity of Bruton Tyrosine kinase in the cells of the subject; alternatively, the preparation is administered to cells in vitro (such as cell lines or cells from subjects) to reduce or inhibit the activity of Bruton Tyrosine kinase in cells in vitro.

In some embodiments, the cells are selected from tumor cells (such as solid tumor cells, including lung cancer cells, breast cancer cells, prostate cancer cells, stomach cancer cells, liver cancer cells, pancreatic cancer cells, ovarian cancer cells, and colon cancer cells).

In some embodiments, the cells are selected from myeloid cells or lymphocytes.

In some embodiments, the cells are primary cells or their cultures from the subject, or established cell lines.

Another aspect of this application relates to a method for reducing or inhibiting the activity of Bruton Tyrosine kinase in cells, including using an effective amount of the compound, its pharmaceutically acceptable salt, its stereoisomer or its prodrug in this application to the cells.

In certain embodiments, the method is performed in vivo or in vitro; preferably, the method is carried out in vivo, for example, applied to the subject (such as the mammal; including bovine, equine, ovine, swine, canine, feline and rodent; and the primate; such as human) to reduce or inhibit the activity of Bruton Tyrosine kinase in the cells of subjects; alternatively, the method is carried out in vitro, for example, applied to cells in vitro (such as cell lines or cells from subjects) to reduce or inhibit the activity of Bruton Tyrosine kinase in cells in vitro.

In some embodiments, the cells are selected from tumor cells (such as solid tumor cells, including lung cancer cells, breast cancer cells, prostate cancer cells, stomach cancer cells, liver cancer cells, pancreatic cancer cells, ovarian cancer cells, and colon cancer cells).

In some embodiments, the cells are selected from myeloid cells or lymphocytes.

In some embodiments, the cells are primary cells or their cultures from the subject, or established cell lines.

Another aspect of this application relates to a kit, which includes the compound of this application, its pharmaceutically acceptable salt, its stereoisomer or its prodrug, and optionally also includes instructions for use.

In some embodiments, the kit is used for reducing or inhibiting the activity of Bruton Tyrosine kinase in cells.

In some embodiments, the cells are selected from tumor cells (such as solid tumor cells, including lung cancer cells, breast cancer cells, prostate cancer cells, stomach cancer cells, liver cancer cells, pancreatic cancer cells, ovarian cancer cells, and colon cancer cells).

In some embodiments, the cells are selected from myeloid cells or lymphocytes.

In some embodiments, the cells are primary cells or their cultures from the subject, or established cell lines.

It is apparent that many other forms of modifications, replacements or changes may be made according to the above content of the present disclosure as well as the common technical knowledge and commonly used means in the field, without departing from the basic technical ideas of the present disclosure.

The above content of the present disclosure will be further explained in detail below through the specific implementation methods of the implementation form. However, this should not be understood as the scope of the above-mentioned subject matter of the present invention being limited to the following examples. All technologies implemented based on the above content belong to the scope of the present disclosure.

### DETAILED DESCRIPTION OF THE EMBODIMENTS

### Chemical Terms

Unless stated otherwise, the following terms are used in the description and claim.

The representation “Cx y” with the following meaning and used in this article represents the range of carbon atoms, where x and y are integers. For example, C₃₋₈ cycloalkyl represents a cycloalkyl group with 3-8 carbon atoms, that is, a cycloalkyl group with 3, 4, 5, 6, 7, or 8 carbon atoms. It should also be understood that ‘C₃₋₈’ also includes any sub range therein, such as C₃₋₇, C₃₋₆, C₄₋₇, C₄₋₆, C₅₋₆, etc.

“Alkyl” refers to a straight or branched hydrocarbon group containing 1 to 20 carbon atoms, such as 1 to 18 carbon atoms, 1 to 12 carbon atoms, 1 to 8 carbon atoms, 1 to 6 carbon atoms, or 1 to 4 carbon atoms. Non limiting examples of alkyl groups include methyl, ethyl, n-propyl, isopropyl, n-butyl, isobutyl, tert butyl, sec-butyl, n-amyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl 1,3-dimethylbutyl and 2-ethylbutyl. The alkyl group can be substituted or unsubstituted.

“Alkenyl” refers to a straight or branched hydrocarbon group containing at least one carbon-carbon double bond and typically 2 to 20 carbon atoms, such as 2 to 8 carbon atoms, 2 to 6 carbon atoms, or 2 to 4 carbon atoms. Non limiting examples of alkenyl groups include vinyl, 1-propenyl, 2-propenyl, 1-Butene, 2-butenyl, 3-butenyl, 2-methyl-2-propenyl, 1,4-pentadienyl and 1,4-butadiene. The alkenyl group can be substituted or unsubstituted.

“Alkynyl” refers to a straight or branched hydrocarbon group containing at least one carbon carbon triple bond and typically 2 to 20 carbon atoms, such as 2 to 8 carbon atoms, 2 to 6 carbon atoms, or 2 to 4 carbon atoms. Non limiting examples of alkynyl groups include acetylene, 1-propargyl, 2-propargyl, 1-butyrgyl, 2-butyrgyl, and 3-butyrgyl groups. The alkynyl group can be substituted or unsubstituted.

“Cycloalkyl” refers to a saturated cyclic hydrocarbon substituent group containing 3 to 14 carbon ring atoms. Cycloalkyl groups can be single carbon rings, typically containing 3 to 7 carbon ring atoms. Non limiting examples of monocyclic cycloalkyl groups include cyclopropyl, cyclobutyl, cyclopentyl, cyclohexyl, and cycloheptyl. The cycloalkyl group can optionally be a fused double or triple ring, such as decahydronaphthyl, and the cycloalkyl group can be substituted or unsubstituted.

“Heterocyclic group”, “heterocyclic alkyl” and “heterocyclic ring” refer to stable 3-18 unit price non aromatic ring, including 2-12 carbon atoms and 1-6 heteroatoms selected from nitrogen, oxygen and sulfur. Unless otherwise specified, heterocyclic groups can be single ring, double ring, triple ring, or quadruple ring systems, which may include fused ring, helical ring, or bridging ring systems. Nitrogen, carbon, or sulfur on heterocyclic groups can be selectively oxidized, nitrogen atoms can be selectively quaternized, and heterocyclic groups can be partially or completely saturated. Heterocyclic groups can be connected to the rest of the molecule through a single bond through carbon or heteroatoms on the ring. Heterocyclic groups containing fused rings can contain one or more aromatic rings or heteroaromatic rings, as long as the atoms on the non aromatic ring are connected with the rest of the molecule. For the purpose of this application, the heterocyclic group preferably consists of a stable 4-11 membered monovalent non aromatic single ring or two rings, comprising 1-3 heteroatoms selected from nitrogen, oxygen, and sulfur, and more preferably a stable 4-8 membered monovalent non aromatic single ring, comprising 1-3 heteroatoms selected from nitrogen, oxygen, and sulfur. Non limiting examples of heterocyclic groups include azacycloheptyl, azacyclobutyl, decahydroisoquinolinyl, dihydrofuranyl, dihydroindolyl, dioxolanyl, 1,1-dioxo-thiomorpholinyl, imidazolinyl, imidazolinyl, isothiazolyl, isoxazolyl, morpholinyl, octahydroindolyl, octahydroisoindolyl, oxazinyl, guazinyl, guaidinyl, 4-guaidinone, pyranyl, pyrazolyl, pyrrolidinyl Quinazinyl, Quinuclidine, tetrahydrofuranyl, Tetrahydropyran, etc.

“Screw heterocyclic group” refers to a multi ring heterocyclic group consisting of 5 to 20 membered rings that share an atom (called a screw atom) between single rings. One or more ring atoms are selected from nitrogen, oxygen, or heteroatoms of S(O)m (where m is an integer 0 to 2), and the remaining ring atoms are carbon. These electronic systems may contain one or more double bonds, but none of the rings have completely conjugated electronic systems, preferably ranging from 6 to 14 elements, and more preferably from 7 to 10 elements. According to the number of shared screw atoms between rings, spiroalkyl groups are divided into single spiroalkyl groups, double spiroalkyl groups, or multiple spiroalkyl groups, preferably single spiroalkyl and double spiroalkyl groups. More preferably, it is a 4-yuan/4-yuan, 4-yuan/5-yuan, 4-yuan/6-yuan, 5-yuan/5-yuan, or 5-yuan/6-yuan single helix ring base. Non limiting embodiments of spirocyclic groups include:

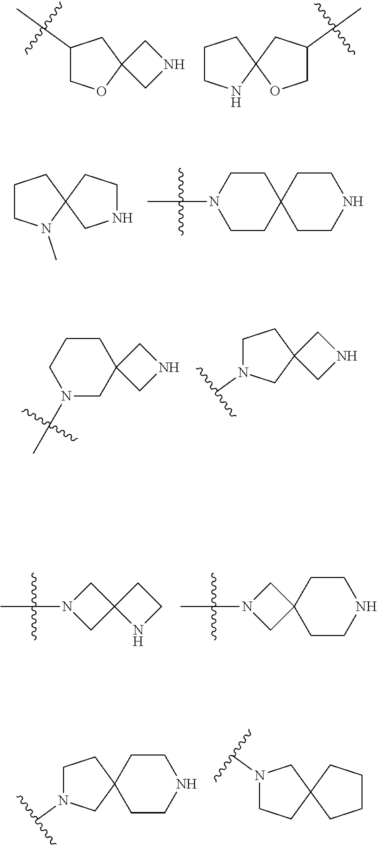

“Fused heterocyclic group” refers to 5 to 20 elements. Each ring in the system shares an adjacent pair of atomic polycyclic heterocyclic group with other rings in the system. One or more rings can contain one or more double bonds, but no ring has a fully conjugated π electronic system. One or more ring atoms are selected from nitrogen, oxygen or S(O)m (where m is an integer 0 to 2), and the remaining ring atoms are carbon. Preferably priced at 6-14 yuan, more preferably priced at 7-10 yuan. According to the number of constituent rings, they can be divided into bicyclic, tricyclic, tetracyclic, or polycyclic fused heterocyclic alkyl groups, preferably bicyclic or tricyclic, and more preferably 5-membered/5-membered or 5-membered/6-membered bicyclic fused heterocyclic groups. Non limiting embodiments of fused heterocyclic groups include:

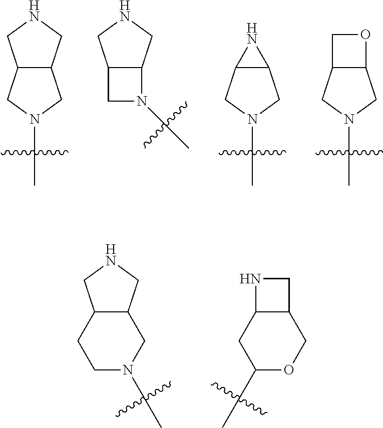

“Aromatic” or “aromatic” refers to aromatic monocyclic or fused polycyclic groups containing 6 to 14 carbon atoms, preferably 6 to 10 elements, such as phenyl and naphthyl, more preferably phenyl. The aromatic ring can be condensed onto a heteroaryl, heterocyclic, or cycloalkyl ring, where the ring connected to the parent structure is an aromatic ring.

“Heteroaryl” or “heteroaryl” refers to a 5-16 membered ring system, which contains 1-15 carbon atoms, preferably 1-10 carbon atoms, 1-4 heteroatoms selected from nitrogen, oxygen and sulfur, and at least one aromatic ring. Unless otherwise specified, heteroaryl groups can be single ring, double ring, triple ring, or four ring systems, which may include fused ring or bridging ring systems. As long as the connection point with other parts of the molecule is an aromatic ring atom, the nitrogen, carbon, and sulfur atoms on the heteroaryl ring can be selectively oxidized, and the nitrogen atoms can be selectively quaternized. For the purpose of the present disclosure, the heteroaryl group preferably is a stable 4-11-membered single aromatic ring, which contains 1-3 heteroatoms selected from nitrogen, oxygen and sulfur, and more preferably is a stable 5-8-membered single aromatic ring, which contains 1-3 heteroatoms selected from nitrogen, oxygen and sulfur. Non limiting examples of heteroaryl groups include acridine group, azapyridyl group, Benzimidazole group, benzoindolyl group, benzodioxin group, benzodioxyl group, Benzofuran ketone group, Benzofuran group, benzonaphthofuranyl group, Benzopyran ketone group, Benzopyran group, benzopyrazolyl group, benzothiadiazole group, Benzothiazole group, Benzotriazole group, furanyl group, imidazolyl group, indozolyl group, indolyl group, oxazole base, purinyl, pyrazinyl Pyrazolyl, pyridazinyl, pyridyl, pyrimidinyl, pyrrolyl, Quinazoline, quinolinyl, quininyl, tetrazolyl, thiadiazole, thiazolyl, thiophenyl, triazinyl, triazolyl, etc. In this application, the heteroaryl group is preferably 5-8-membered heteroaryl group, which includes 1-3 heteroatoms selected from nitrogen, oxygen, and sulfur, more preferably pyridine, pyrimidine, and thiazole groups. The heteroaryl group can be substituted or unsubstituted.

“Halogen” refers to fluorine, chlorine, bromine, or iodine.

“Hydroxyl” refers to —OH, “amino” refers to —NH2, “amide” refers to —NHCO—, “cyano” refers to —CN, “nitro” refers to —CN, “isocyano” refers to —NC, and “trifluoromethyl” refers to —CF3.

The terms “heteroatom” or “heteroatom” used alone or as part of other components in this article refer to atoms other than carbon and hydrogen, which are independently selected from oxygen, nitrogen, sulfur, phosphorus, silicon, selenium, and tin, but are not limited to these atoms. In the embodiments where two or more heteroatoms appear, the two or more heteroatoms may be identical to each other, or some or all of the two or more heteroatoms may be different from each other.

The term “thick” or “thick ring” used alone or in combination in this article refers to a circular structure where two or more rings share one or more bonds.

The term “screw” or “spiral ring” used alone or in combination in this article refers to a circular structure where two or more rings share one or more atoms.

“Optional” or “optionally” means that the subsequent described event or environment can but does not necessarily occur, and this description includes the occurrence or absence of the event or environment. For example, optionally substituted heterocyclic groups with alkyl groups' means that alkyl groups can but do not have to exist, including situations where heterocyclic groups are replaced by alkyl groups and situations where heterocyclic groups are not replaced by alkyl groups.

“Substituted” refers to one or more atoms in a functional group, preferably 5 or 1-3 atoms, independently replaced by a corresponding number of substituents. It goes without saying that substituents are located in their possible chemical positions, and those skilled in the art can determine (through experiments or theory) possible or impossible substitutions without excessive effort. For example, binding free amino or hydroxyl groups to carbon atoms with unsaturated (such as olefins) bonds may be unstable. The substituents include but are not limited to hydroxyl, amino, halogen, cyano, C₁₋₆ alkyl, C₁₋₆ alkoxy, C₂₋₆ alkenyl, C₂₋₆ alkynyl, C₃₋₈ cycloalkyl, etc.

“Pharmaceutical composition” refers to a composition containing one or more of the compounds described herein or their pharmaceutically active acceptable or prodrugs, as well as other components such as pharmaceutically acceptable carriers and excipients. The purpose of the pharmaceutical composition is to promote the administration of drugs to organisms, facilitate the absorption of active ingredients, and thereby exerting biological activity.

“Isomer” refer to a compound with the same molecular formula but different atomic binding properties or orders or different atomic spatial arrangement, and the isomer with different atomic spatial arrangement is called “stereoisomer”. Stereisomer includes an optical isomer, a geometric isomer and a conformational isomer. The compound of the present disclosure can exist in the form of optical isomer. According to the configuration of substituents around chiral carbon atoms, these optical isomers are in the “R” or “S” configuration. The optical isomers include enantiomer and diastereomer, and methods for preparing and separating optical isomers are known in the art.

The compounds of the present disclosure can also have geometric isomers. Various geometric isomers and mixtures thereof generated by the distribution of substituents around carbon-carbon double bonds, carbon nitrogen double bonds, cycloalkyl or heterocyclic groups are considered in the present disclosure. The substituents around carbon-carbon double bonds or carbon nitrogen bonds are designated as Z or E configurations, and the substituents around cycloalkyl or heterocycles are designated as cis or trans configurations.

The compounds of the present disclosure may also exhibit tautomerism, such as Keto-enol tautomerism.

It should be understood that the present disclosure includes any tautomeric or stereoisomeric form and its mixture, and is not limited to any tautomeric or stereoisomeric form used in the naming of compounds or chemical combinations.

“Isotopes” refer to all isotopes of atoms present in compounds of the present disclosure. Isotopes include those atoms with the same atomic number but different mass numbers. Examples of isotopes suitable for incorporation into compounds of the present invention are hydrogen, carbon, nitrogen, oxygen, phosphorus, fluorine, and chlorine, such as but not limited to ²H, ³H , ¹³C , ¹⁴C , ¹⁵N , ¹⁸O, ³¹P, ³²P, ³⁵S, ¹⁸F, and ³⁶Cl, respectively. The isotopic labeling compounds of the present disclosure can usually be prepared by using appropriate isotopic labeling reagents instead of non isotopic labeling reagents through traditional techniques known to those skilled in the art or by methods similar to those described in the attached embodiments. Such compounds have various potential applications, such as serving as standards and reagents for measuring biological activity. In the case of stable isotopes, such compounds have the potential to advantageously alter biological, pharmacological, or pharmacokinetic properties.

“Prodrug” refers to the compound of the present disclosure that can be administered in the form of a prodrug. Prodrugs refer to the derivatives of biologically active compounds invented under physiological conditions in vivo, such as through oxidation, reduction, hydrolysis, etc. (each using enzymes or without enzyme participation). Examples of prodrugs are the following compounds: the amino group in the compound of the present disclosure is acylated, alkylated or phosphorylated, such as icosane acylamino, propylamine amido, pivaloyloxymethyl amino, or the hydroxyl group is acylated, alkylated, phosphorylated or converted to borate, such as acetoxy group, palmitoxy, pivaloyloxy, succinyloxy, fumaroyloxy, propiamoyloxy or the carboxyl group is esterified or amidated, or the mercapto group forms disulfide bridge bonds with carrier molecules that selectively target and/or deliver drugs to the cytosol of cells, such as peptides, these compounds can be prepared by the compounds of the present disclosure according to the well-known methods.

“Medicinal salt” or “pharmaceutically acceptable” refers to a medicinal base or acid, including inorganic base or acid and organic base or acid. In the case where the compound of the present disclosure contains one or more acidic or alkaline groups, the present disclosure also includes their corresponding medicinal salts. Therefore, compounds of the present disclosure containing acidic groups can exist in the form of salts and can be used according to the present disclosure, for example as alkali metal salts, alkali earth metal salts, or as ammonium salts. More exact examples of such salts include sodium salt, potassium salt, calcium salt, magnesium salt or amine or organic amine, such as primary amine, secondary amine, tertiary amine, cyclic amine, etc., such as ammonia, Isopropylamine, Trimethylamine, Diethylamine, triethylamine, Tripropylamine, Ethanolamine, Diethanolamine, Ethanolamine, Dicyclohexylamine, Ethylenediamine, purine, guazine, guaidine, choline, caffeine, and other particularly preferred Organic base are Isopropylamine, Diethylamine, Ethanolamine, Trimethylamine A salt of Dicyclohexylamine, choline, and caffeine. The compounds of the present disclosure containing alkaline groups can exist in salt form and can be used in the form of their addition to inorganic or organic acids according to the present disclosure. Examples of suitable acids include hydrochloric acid, hydrobromic acid, phosphoric acid, sulfuric acid, phosphoric acid, methanesulfonic acid, p-toluenesulfonic acid, naphthalene disulfonic acid, oxalic acid, acetic acid, tartaric acid, lactic acid, salicylic acid, benzoic acid, formic acid, propionic acid, tervaleric acid, malonic acid, succinic acid, pimelic acid, fumaric acid, maleic acid, malic acid, amino sulfonic acid, phenylpropionic acid, gluconic acid, ascorbic acid, isonicotinic acid, citric acid adipic acid and other acids known to those skilled in the art. If the compound of the present disclosure contains both acidic and alkaline groups in the molecule, in addition to the salt form mentioned, the present disclosure also includes inner salts or inner ammonium salts. The salts are obtained by conventional methods known to those skilled in the art, such as by contacting these with organic or mineral acid or bases in solvents or dispersants, or by anion exchange or cation exchange with other salts.

Therefore, when referring to “compound”, “compound of the present disclosure” or “compound of the present disclosure” in this application, all the compound forms, such as its prodrug, stable isotope derivatives, medicinal salts, isomers, racemates, racemic mixture, enantiomer, diastereomers and their mixtures are included.

In this context, the term “tumor” includes benign tumor and malignant tumor (such as cancer).

In this context, the term “cancer” includes various malignant tumors in which Bruton's Tyrosine kinase is involved, including but not limited to non-small cell lung cancer, esophageal cancer, Melanoma, striated muscle pomegranate, cell cancer, Multiple myeloma, breast cancer, ovarian cancer, uterine membrane cancer, cervical cancer, gastric cancer, colon cancer, bladder cancer, pancreatic cancer, lung cancer, breast cancer, prostate cancer and liver cancer (such as hepatocellular carcinoma), more specifically liver cancer Gastric cancer and bladder cancer.

The terms “effective amount”, “therapeutic effective amount”, or “pharmaceutical effective amount” used in this article refer to the amount of at least one medication or compound that is sufficient to alleviate one or more symptoms of the treated disease or condition to some extent after administration. The result may be a reduction and/or remission of signs, symptoms or causes or any other desired change in the biological system. For example, the “effective amount” used for treatment is the amount of composition containing the compounds disclosed in this article required to provide significant symptom relief effects in clinical practice. Techniques such as dose escalation testing can be used for determining the effective amount suitable for any individual case.

The term “polycrystalline form” or “polycrystalline form (phenomenon)” used in the present disclosure refers to the compound of the present disclosure having multiple crystal lattice forms. Some compounds of the present disclosure may have more than one crystal form, and the present disclosure covers all polycrystalline forms or mixtures thereof.

The intermediate compound and its multiple forms of the compound of the present disclosure are also within the scope of the present disclosure.

Crystallization often produces solvate of the compound of the present disclosure. The term “solvate” used herein refers to a combination of one or more compound molecules of the present disclosure and one or more solvent molecules.

The solvent can be water, in this case, the solvate is hydrate. Alternatively, it can be an organic solvent. Therefore, the compounds of the present disclosure can exist as hydrates, including monohydrate, dihydrate, hemihydrate, trihydrate, tetrahydrate, etc., and corresponding solvation forms. The compounds of the present disclosure can be true solvate, but in other cases, the compounds of the present disclosure may only occasionally retain water or a mixture of water and some other solvents. The compounds of the present disclosure can react in a solvent or precipitate or crystallize in a solvent. The solvate of the compound of the present disclosure is also included in the scope of the present disclosure.

The term “acceptable” used in this article in relation to formulations, compositions, or ingredients refers to the absence of sustained harmful effects on the overall health of the treatment subject.

The term “pharmaceutically acceptable” used in this article refers to a substance (such as a carrier or diluent) that does not affect the biological activity or properties of the compound of the present disclosure and is relatively non-toxic, meaning that the substance can be applied to an individual without causing adverse biological reactions or interacting with any component contained in the composition in an adverse manner.

“Pharmaceutically acceptable carriers” include but are not limited to adjuvants, carriers, excipients, additives, deodorants, diluents, preservatives, dyes/colorants, flavor enhancers, surfactants and wetting agents, dispersants, suspensions, stabilizers, and other penetrating agents, solvents, or emulsifiers that have been approved by relevant government administrative departments for use in humans and domesticated animals.

The terms “subject,” “patient,” “object,” or “individual” used in the text refer to individuals suffering from diseases, disorders, or illnesses, including mammals and non mammals. Examples of mammals include but are not limited to any member of the mammalian class: humans, non human primates (such as chimpanzees and other apes and monkeys); livestock, such as cows, horses, sheep, goats, pigs; domestic animals, such as rabbits, dogs, and cats; laboratory animals, including rodents such as rats, mice, and guinea pigs. Examples of non-human mammals include but are not limited to birds and fish. In an embodiment of the method and composition provided in this article, the mammal is a human.

The term “treatment” used in this article refers to the treatment of related diseases and conditions in mammals, especially humans, including
(i) Prevent mammals, especially those who have been previously exposed to a disease or condition but have not been diagnosed with that disease or condition, from developing the corresponding disease or condition;
(ii) Suppress the disease or symptom, i.e. control its development;
(iii) Alleviate the disease or condition, that is, slow down the resolution of the disease or condition;
(iv) Relieve symptoms caused by diseases or symptoms.

The terms “disease” and “disease” used in this article can be substituted for each other or have different meanings, as certain specific diseases or diseases do not yet have known pathogenic factors (so the cause of the disease is not yet clear), so they cannot be recognized as diseases and can only be seen as unwanted conditions or syndromes. The specific symptoms of these syndromes have been confirmed by clinical researchers to some extent.

The terms “take”, “apply”, “administration”, etc. used in this article refer to methods that can deliver a compound or composition to the desired site for biological action, including but not limited to oral route, duodenal route, parenteral injection (including intravenous, subcutaneous, intraperitoneal, intramuscular, arterial injection or infusion), local administration, and rectal administration. In the preferred embodiment, the compounds and compositions discussed in this article are administered orally.

### Synthetic Method

The present disclosure also provides a method for preparing the compound. The preparation of the compound described in general formula (I) of the present disclosure can be achieved through the following exemplary methods and examples, but these methods and examples should not be considered in any way as limiting the scope of the present disclosure. The compounds described in the present disclosure can also be synthesized using synthesis techniques known to those skilled in the art, or by combining known methods in the art with the methods described in the present disclosure. The products obtained in each step should be obtained using known separation techniques in the field, including but not limited to extraction, filtration, distillation, crystallization, chromatographic separation, etc. The starting materials and chemical reagents required for synthesis can be conventionally synthesized or purchased according to literature (reaxys).

The compound of the present disclosure can be obtained using the following solutions:

### Solution A:

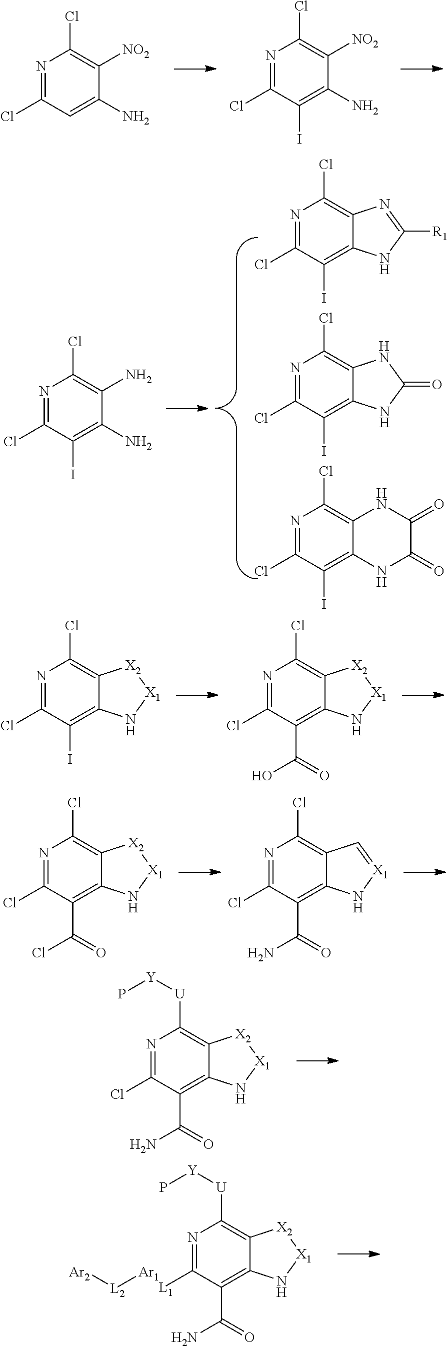

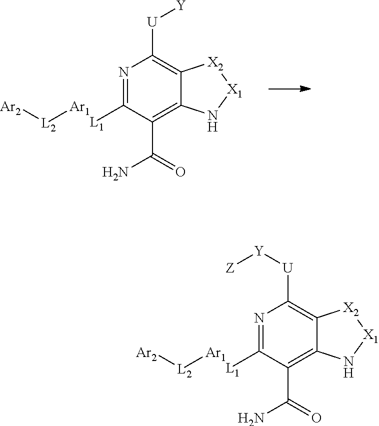

### Solution B:

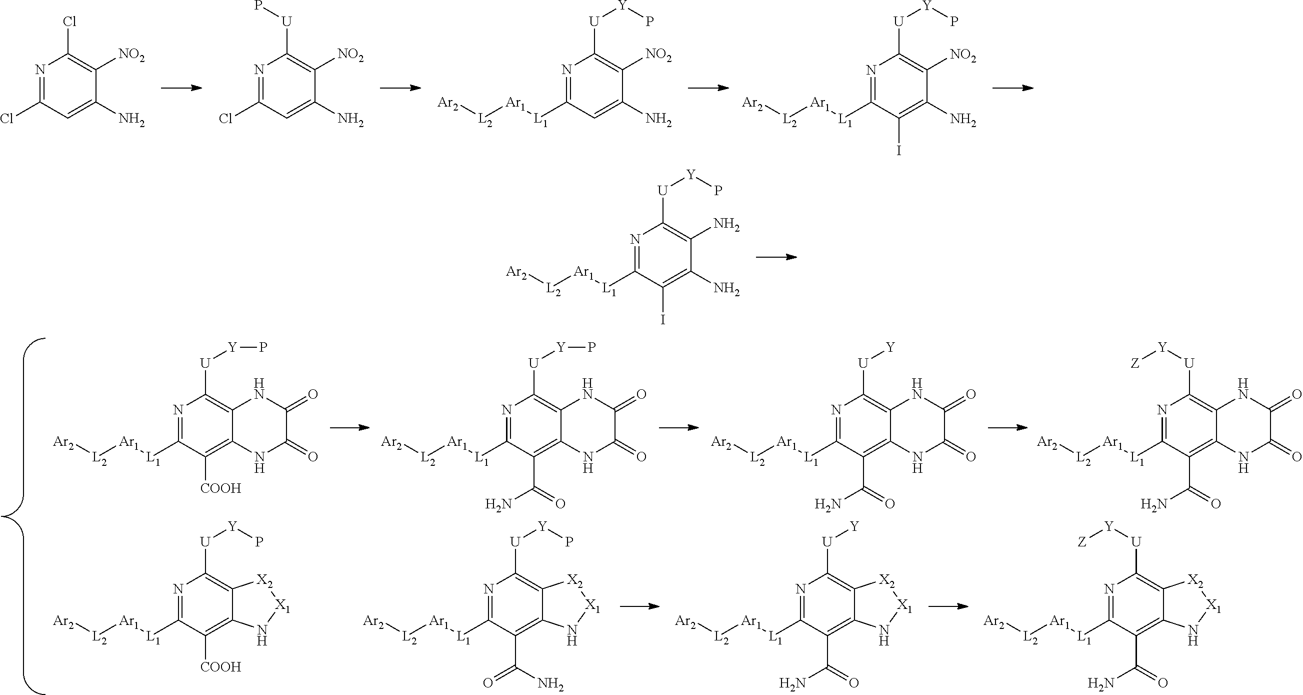

### Solution C:

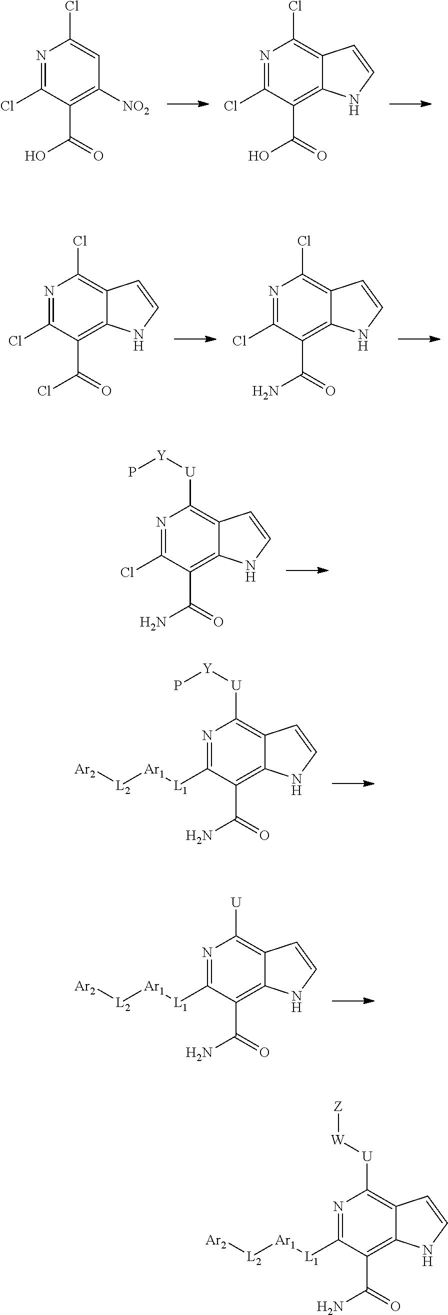

### Solution D:

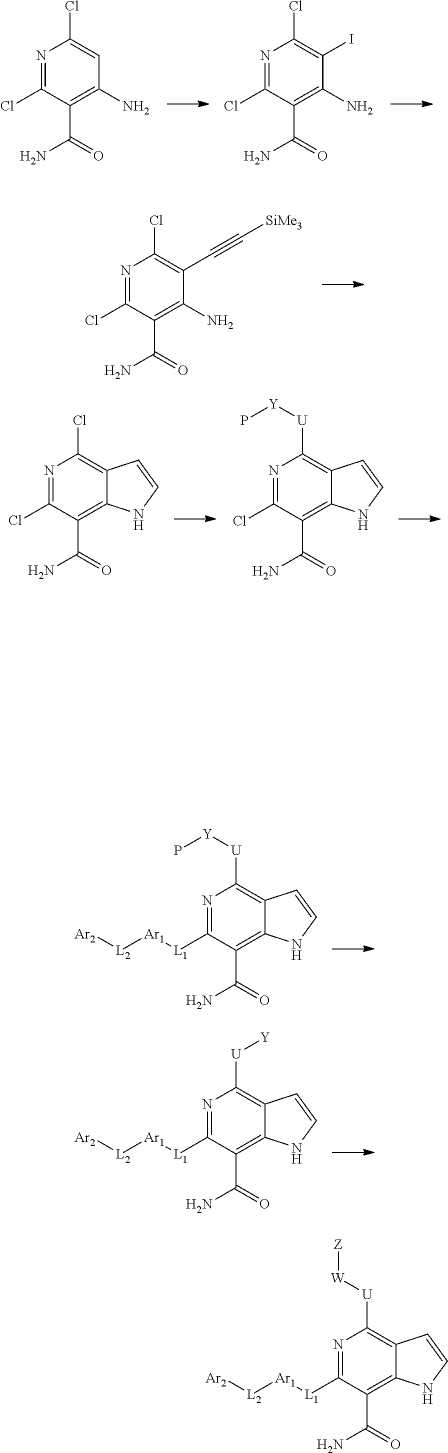

### Solution E:

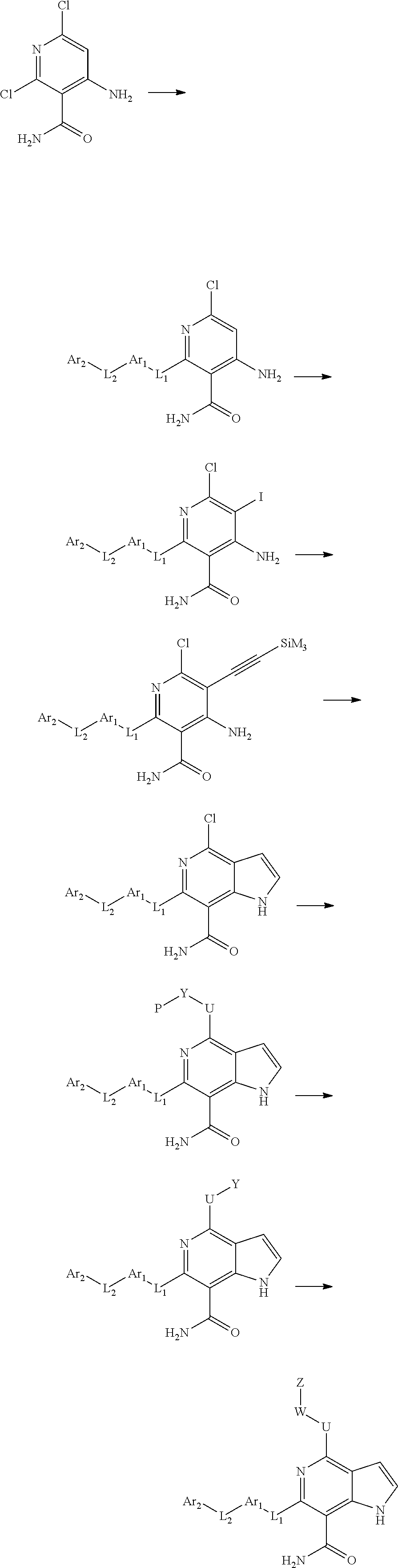

### Solution F:

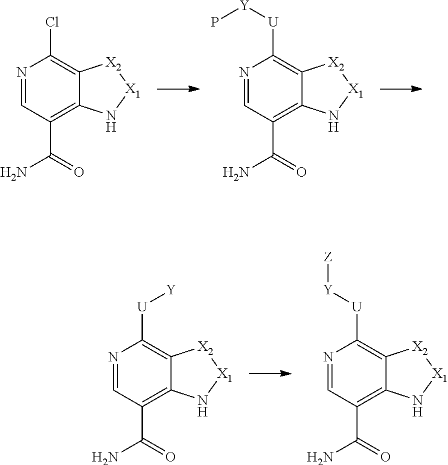

Unless otherwise specified, the temperature is in Celsius. The reagents are purchased from commercial suppliers such as Chem blocks Inc. or Sunway pharm Inc, and these reagents can be used directly without further purification, unless otherwise specified.

Unless otherwise specified, the following reactions shall be carried out at room temperature, in anhydrous solvent, under the positive pressure of nitrogen or gas or using a drying tube; dry and/or heat dry glassware.

Unless otherwise specified, column chromatography purification uses 200-300 mesh silica gel from Qingdao Ocean Chemical Plant; the thin layer chromatography silica gel prefabricated board (HSGF254) produced by Yantai Chemical Industry Research Institute is used for the preparation of thin layer chromatography; the determination of MS is performed using Therno LCD Fleet (ESI) liquid chromatography-mass spectrometry.

The Bruker Avance-400 MHz or Varian Oxford 400 Hz nuclear magnetic spectrometer is used for nuclear magnetic data (1H NMR). The solvents used for nuclear magnetic data are CDCl₃, CD₃OD, D₂O, DMS-d₆, etc., with Tetramethylsilane (0.000 ppm) as the benchmark or residual solvent as the benchmark (CDCl₃: 7.26 ppm; CD₃OD: 3.31 ppm; D₂O: 4.79 ppm; d6-DMSO: 2.50 ppm). When the diversity of peak shapes is indicated, the following abbreviations indicate different peak shapes: s (single peak), d (double peak), t (triple peak), q (quadruple peak), M (multiple peak), br (wide peak), dd (double-double peak), dt (double triple peak). If the coupling constant is given, it is in Hertz (Hz).

### Preparation for a Key Intermediate: 4,6-dichloro-2-oxo-imidazo[4,5-c]pyridine-7-carboxamide

NBS (5.2 g, 29.6 mmol) was added to a solution of 4,6-dichloro-2-oxo-imidazo[4,5-c]pyridine in acetic acid (100 mL), the acetic acid was removed under pressure reduction after stirring 2 h at 60° C., the residue was suspended in the water (60 mL), and a saturated sodium bicarbonate solution (40 mL) was added. The solid was filtered and stirred for 30 min in 80° C. of water (200 mL). After cooling to an environment temperature, the solid was filtered and dried in vacuum, so as to obtain a crude product: 7.11 g of 4,6-dichloro-6-br-2-oxo-imidazo[4,5-c]pyridine, with a yield 93%. LC/MS(ESI): m/z=283[M+H]⁺.

N,N,N′,N′-tetramethylethylenediamine (9 g, 50.1 mmol) was added to the product 4,6-dichloro-6-br-2-oxo-imidazo[4,5-c]pyridine (5.64 g, 20 mmol) in the previous step and stored in an anhydrous tetrahydrofuran (100 mL) solution, and the solution was cooled to −60° C. and stirred for 1 min under nitrogen atmosphere. ⁿBuLi (20.4 mL, 50.1 mmol, 2.5M in hexane) was added dropwise to the solution, then the mixture was stirred for 2 h. Dry carbon dioxide gas was bubbled into the solution and the mixture was stirred for 1 h at −60° C. After warming to room temperature, water (100 mL) was added. The tetrahydrofuran was removed through pressure relief, and the residue was distributed in ethyl acetate and water. The water layer was acidized to pH=1 with 1M of hydrochloric acid, the solid was filtered to obtain a crude product: 3.77 g of 4,6-dichloro-2-oxo-imidazo[4,5-c]pyridine-7-formic acid, with a yield 76%. LC/MS(ESI): m/z=249[M+H]⁺.

The 4,6-dichloro-2-oxo-imidazo[4,5-c]pyridine-7-formic acid (2.49 g, 10 mmol) was dissolved in thionyl chloride (40 ml), the mixture was heated to 75° C. and stirred for 2 h. The redundant thionyl chloride was removed in vacuum, the residue was dissolved in dry THF (40 mL). After cooling to 0° C., ammonium water (6 mL) was added, the mixture was stirred for 10 h under the environment temperature. The solid was filtered and recrystallized from ethyl (20 mL) so as to obtain 2.08 g of 4,6-dichloro-2-oxo-imidazo[4,5-c]pyridine-7-carboxamide, with a yield 84%. LC/MS(ESI): m/z=248[M+H]⁺.

### Preparation for a Key Intermediate: 4,6-dichloroprazole[4,5-c]pyridine-7-carboxamide

NBS (5.2 g, 29.6 mmol) was added to a solution of 2,6-dichloro-3-methylpyridin-4-amine (17.7 g, 0.1 mol) in acetic acid (300 mL), the acetic acid was removed under pressure reduction after stirring 2 h at 60° C., the residue was suspended in the water (180 mL), and a saturated sodium bicarbonate solution (120 mL) was added. The solid was filtered and stirred for 30 min in 80° C. of water (600 mL). After cooling to an environment temperature, the solid was filtered and dried in vacuum, so as to obtain a crude product: 23.8 g of 2,6-dichloro-3-methylpyridin-4-amino-5-picoline, with a yield 93%. LC/MS(ESI): m/z=256[M+H]⁺.

The 2,6-dichloro-3-methylpyridin-4-amino-5-picoline (12.8 g, 0.05 mol) obtained in the previous step was dissolved in acetic anhydride (50 mL) and anhydrous toluene (500 mL), the mixture was heated to 100° C. and stirred for 14 h, then the mixture was cooled to the room temperature, desolventized through pressure relief, so as to obtain the intermediate crude product: 2,6-dichloro-3-methylpyridin-4-amino-5-picoline, which can be directly used for the next reaction without purification. LC/MS(ESI): m/z=256[M+H]⁺.

The crude products: 2,6-dichloro-3-methylpyridin-4-amino-5-picoline, acetic anhydride, potassium acetate and methylbenzene in the previous step were heated to 78° C., isoamyl nitrite was added while still hot to be stirred for 18 h, the mixture was cooled to the room temperature and desolventized through pressure relief, and the intermediate crude product was obtained.

N,N,N′,N′-tetramethylethylenediamine (9 g, 50.1 mmol) was added to the product 4,6-dichloro-6-bromopyrazole[4,5-c]dipyridine (5.64 g, 20 mmol) in the previous step and stored in an anhydrous tetrahydrofuran (100 mL) solution, and the solution was cooled to −60° C. and stirred for 1 min under nitrogen atmosphere. nBuLi (20.4 mL, 50.1 mmol, 2.5M in hexane) was added dropwise to the solution, then the mixture was stirred for 2 h. Dry carbon dioxide gas was bubbled into the solution and the mixture was stirred for 1 h at −60° C. After warming to room temperature, water (100 mL) was added. The tetrahydrofuran was removed through pressure relief, and the residue was distributed in ethyl acetate and water. The water layer was acidized to pH=1 with 1M of hydrochloric acid, the solid was filtered to obtain a crude product: 3.77 g of 4,6-dichloroprazole[4,5-c]pyridine-7-formic acid, with a yield 76%. LC/MS(ESI): m/z=249[M+H]⁺.

4,6-dichloropyrazolimidazole[4,5-c]pyridine-7-formic acid (2.49 g, 10 mmol) was dissolved in thionyl chloride (40 ml), the mixture was stirred for 2 h at 75° C. The redundant thionyl chloride was removed in vacuum, the remnant was dissolved in anhydrous tetrahydrofuran (40 mL). Ammonium hydroxide (6.0 mL) was added at 0° C., and the mixture was stirred for 10 H in the environment temperature. The solid was filtered and recrystallized from ethyl alcohol (20 mL) so as to obtain 2.08 g of 4,6-dichloro-2-oxo-imidazo[4,5,c]pyridine-7-carboxamide, with a yield 84%. LC/MS(ESI): m/z=248[M+H]⁺.

### Preparation for a Key Intermediate: 4,6-dichloro-pyrrole[3,2-c]dipyridine-7-carboxamide

ⁿBuLi (27.8 mL, 69.6 mmol, 2.5M in hexane) was added to diisopropylamine (7.5 g, 74.3 mml) at −78° C. and dissolved in the solution of tetrahydrofuran (50 mL), then the mixture was stirred for 30 min at −78° C. The solution that the 2,6-dichloro-nitropyridine (19.0 g, 67.6 mmol) was stored in the tetrahydrofuran (50 mL) was added during 40 min, the mixture was stirred for 3 h at −78° C. Dry carbon dioxide gas was bubbled into the reaction mixture stirred and overnight at the room temperature. The solvent was removed under pressure relief, and the residue was distributed in the mixture of ethyl acetate (50 mL) and 10% sodium hydroxide water solution (100 mL). The concentrated hydrochloric acid made the water be acidic, extraction was carried outer through the ethyl acetate (3×150 mL). The organic layer was dried on sodium sulfate, filtered and concentrated to obtain the intermediate: 2,6-dichloro-4-nitronicotinic acid, without further purification. LC/MS(ESI): m/z=326[M+H]⁺.

2,6-dichloro-4-nitronicotinic acid (49 g, 154 mmol) was dissolved in anhydrous THF (1000 mL) and stirred for about 5 mm after cooled to −40° C. and −50° C., and then vinylmagnesium bromide (692 mL and 692 mmol in THF) was gradually added. The mixture was stirred for about 4 h between −40° C. and −50° C. The reactant was quenched by a saturated NH₄Cl water solution (20 mL). The solvent was removed under pressure relief so as to obtain the residue. The 4,6-dibromo-1H-pyrrole[3,2-c]pyrrole-7-carboxamide (5.4 g) was obtained by preparing the HPLC purification, with a yield 11%. LC/MS(ESI): m/z=321[M+H]⁺.

HOBt (2.29 g, 15 mmol) and EDCl (2.88 g, 15 mmol) were added to the solution that 4,6-dichloro-1H-pyrrole[3,2-c]pyrrole-7-carboxamide (3.2 g, 1 mmol) was stored in DMF (50 mL). After the reaction mixture was stirred for about 1 H at the room temperature, NH₃/THF (200 mL) was added, and the obtained mixture was stirred and overnight at the room temperature. The suspension liquid was filtered, and then the filtrate was depressurized and concentrated. Extraction was carried out by adding water and ethyl acetate. The combined organic phase was washed by saline and dried by Na₂SO₄, and 4,6-dibromo-1H-pyrrole[3,2-c]pyrrole-7-carboxamide (1.53 g, 48%) was provided through filtering, depressurization and concentration. LC/MS(ESI): m/z=320[M+H]⁺.

### Synthesis of a Key Intermediate: 4-chloro-2-oxyimidazole[4,5,c]dipyridine-7-formonitrile

By adopting the method of the literature (Chemistry of Heterocyclic Compounds, 1994, vol. 30, #8, p. 923-927) and when cooled by the cooling water, the solution that 7-br-2-oxyimidazole[4,5,c]dipyridine (5.35 g, 25 mmol) was dissolved in 15 mL of concentrated sulfuric acid was dropped to the solution that nitrate (7 g, 69 mmol) was dissolved in 15 mL of concentrated sulfuric acid. The mixture was heated to 100° C., stirred for 2 h, cooled to the room temperature, poured to ice water and neutralized to pH=6 with a sodium hydroxide water solution. The solid was filtered and dried in vacuum, and then crystallized with DMF to obtain a brown yellow solid 7-bromo-4-nitro-1,3-dihydro-2H-imidazo[4,5-c]pyridin-2-one (4.92 g, yield 76%). LC/MS(ESI): m/z=260[M+H]⁺.

7-bromo-4-nitro-1,3-dihydro-2H-imidazo[4,5-c]pyridin-2-one (2.6 g, 10 mmol) and 50 mL concentrated hydrochloric acid were put into a sealed tube, heated to 150-160° C., stirred for 12 h, cooled to room temperature, reduced of pressure to remove water, then 10 mL of water was added, neutralized with sodium bicarbonate aqueous solution to pH=6, stirred for half an hour, the solid is filtered and dried under vacuum to obtain a brown solid 7-bromo-4-chloro-1,3-dihydro-2H-imidazo[4,5-c]pyridin-2-one (2.0 g, yield 81%). LC/MS(ESI): m/z=249[M+H]⁺.

The compound 7-bromo-4-chloro-1,3-dihydro-2H-imidazo[4,5-c]pyridin-2-one (1.98 g, 8.0 mmol), zinc cyanide (0.56 g, 4.8 mmol), 10 wt % Pd/C (0.36 g, 0.16 mmol), 1,1′-bis (diphenylphosphine) ferrocene (0.18 g, 0.32 mmol) and N,N-dimethylacetamide (15 ml) were added to the reaction flask. Replace with nitrogen for three times, add zinc formate dihydrate (0.15 g, 0.8 mmol), replace with nitrogen again for three times, and react at 100° C. overnight. Cool to room temperature, dilute the reaction solution with ethyl acetate and water, and extract with ethyl acetate. The obtained organic phase was washed with water and saturated salt water, dried with anhydrous sodium sulfate, and evaporated to dryness under reduced pressure. The residue was purified by column chromatography to obtain the compound 4-chloro-2-oxo-2,3-dihydro-1H-imidazo[4,5-c]pyridine-7-carbonitrile (1.21 g, yield 78%) as a brown solid. LC/MS(ESI): m/z=195[M+H]⁺.

### Example 1

### (S)-4-(3-(but-2-ynamido)piperidin-1-yl)-2-oxo-6-(4-phenoxyphenyl)-2,3-dihydro-1H-imidazo[4,5-c]pyridine-7-carboxamide (Compound 1)

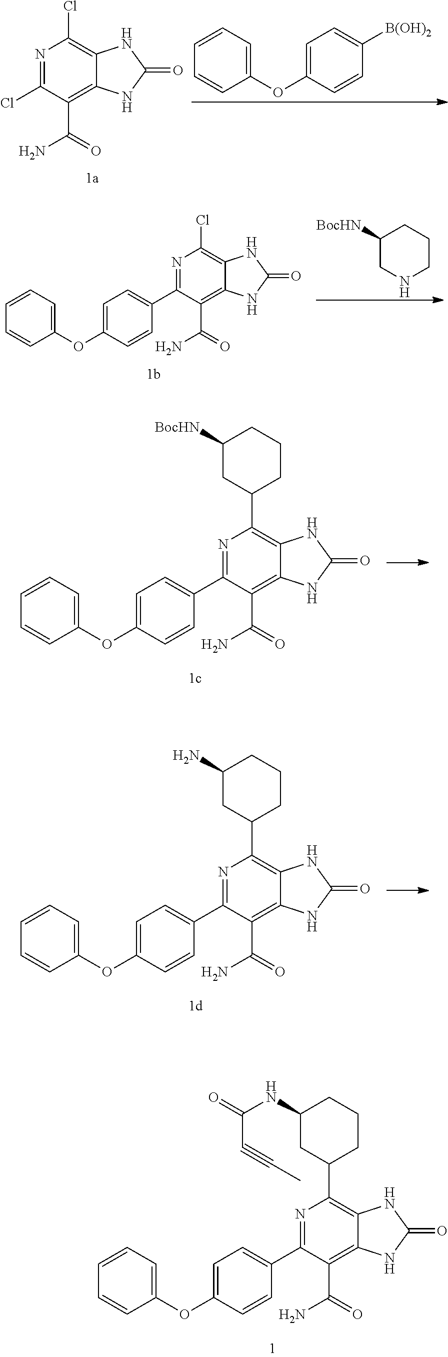

### Step 1: Synthesis of Compound 1b

4,6-dichloro-2-oxo-2,3-dihydro-1H-imidazo[4,5-c]pyridine-7-carboxamide 1a (0.74 g, 3 mmol), 4-phenoxyphenylboronic acid (1.32 g, 6 mmol) and tri-potassium phosphate monohydrate were dissolved in dioxane (10 mL) and water (4 mL), Pd(PPh₃)₄ (0.53 g, 0.45 mmol) was added after charging nitrogen for many times, the mixture was sprayed for 5 min with the nitrogen, and refluxed and heated for 24 h. The reactant was cooled to room temperature and stirred overnight to produce a yellowish precipitate, the reaction mixture was diluted with water (10 mL), and the solid was collected through filtration. The crude product was pulped with methanol (50 mL), and then an off-white solid (0.66 g, 58%) was obtained. The next reaction was carried out without further purification, LC/MS(ESI): m/z=382 [M+H]⁺.

### Step 2: Synthesis of Compound 1c

The product 1b (0.38 g, 1 mmol) obtained the previous step, (S)-3-Boc-aminopiperidine (0.22 g, 1.1 mmol), potassium carbonate (0.22 g, 2 mmol) catalytic amount of potassium iodide and DMF (30 mL) were mixed. The mixture was heated to 120° C. and stirred for 4 h. After cooling to room temperature, the mixture was depressurized and dissolved. The residue was purified by flash column to afford a yellow solid 1c (0.315 g, 58%). LC/MS(ESI): m/z=545.2[M+H]⁺.

### Step 3: Synthesis of Compound 1d

The intermediate 1c (0.27 g, 0.5 mmol) in the previous step, 2 mL of ethyl acetate and 4 ml of IN HCl 1.4-dioxane solution were added to the reaction flask. The mixture was stirred for 2 h at room temperature. The solution was neutralized with 1N aq. NaOH, extracted with ethyl acetate. The obtained organic phase was washed with saturated sodium bicarbonate and saturated saline solution, dried with anhydrous sodium sulfate, and then the organic phase was depressurized and dried by distillation so as to obtain the compound 1d (0.193 g, yield 87%), it can be directly used for the next step, LC/MS(ESI): m/z=445.2[M+H]⁺.

### Step 4: Synthesis of Compound 1

The compound 1d (133 mg, 0.3 mmol), TEA (51 mg, 0.5 mmol) and 4 ml of tetrahydrofuran were added to the reaction flask, after cooled in an ice bath, 0.5 ml of tetrahydrofuran solution of but-2-ynoyl chloride (45 mg, 0.5 mmol) was dropped. After dropping, the solution was continuously stirred for 4 h. The reaction liquid was quenched through methyl alcohol for reaction and depressurized and evaporated. The residue was purified through column chromatography, so as to obtain the compound I (49 mg, yield 32%) as a yellow solid. ¹H NMR (400 MHz, CD3OD) δ: 7.41-7.04 (m, 9H), 3.98-3.83 (m, 1H), 3.19-3.07 (m, 1H), 3.06-2.89 (m, 1H), 3.06-2.91 (m, 1H), 2.80 (m, 1H), 2.80 (br s, 1H), 1.96 (s, 3H); 1.91-1.80 (m, 1H), 1.73 (s, 2H) and 1.35 (s, 1H); LC/MS(ESI): m/z=511.2[M+H]⁺.

### Example 2

### (S)-4-(3-(but-2-ynamido)piperidin-1-yl)-6-(4-phenoxyphenyl)-1H-pyrazolo [4,5-c]pyridine-7-carboxamide (Compound 2)

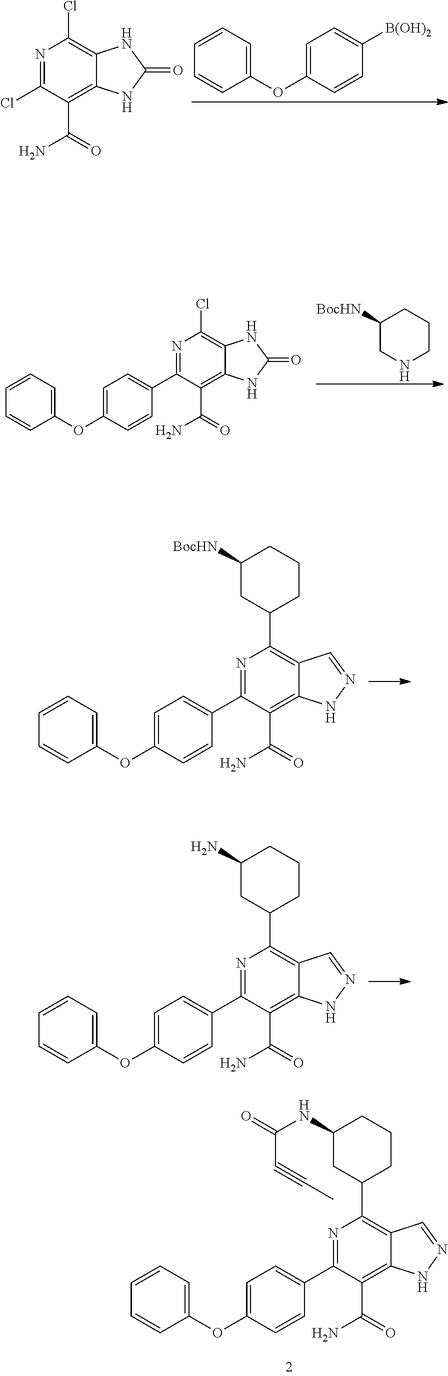

The compound 2 (53 mg, yield 36%, which was the yield of the final step, the same below) obtained by a procedure analogous to the procedure described in Example 1 (the intermediate was replaced to 4,6-dichloro-2-oxo-2,3-dihydro-1H-imidazo[4,5-c]pyridine-7-carboxamide) was a light yellow solid. ¹H NMR (400 MHz, CD3OD) δ: 7.56-7.04 (m, 9H), 3.98-3.84 (m, 1H), 3.19-3.07 (m, 1H), 3.06-2.89 (m, 1H), 3.06-2.91 (m, 1H), 2.80 (br s, 1H), 1.98 (s, 3H), 1.91-1.80 (m, 1H), 1.73 (s, 2H) and 1.36 (s, 1H); LC/MS(ESI): m/z=495[M+H]⁺.

### Example 3

### (S)-4-(3-acrylamidopiperidin-1-yl)-2-oxo-6-(4-phenoxyphenyl)-2,3-dihydro-1H-imidazo[4,5-c]pyridine-7-carboxamide (Compound 3)

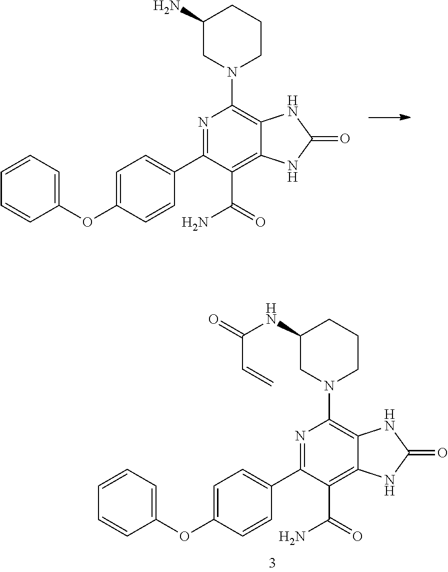 [SMILES: NC(=O)c1c(-c2ccc(Oc3ccccc3)cc2)nc(N2CCC[C@H](N)C2)c2[nH]c(=O)[nH]c12 ; C=CC(=O)N[C@H]1CCCN(c2nc(-c3ccc(Oc4ccccc4)cc3)c(C(N)=O)c3[nH]c(=O)[nH]c23)C1]

The compound 2 (48 mg, yield 32%, which was the yield of the final step, the same below) obtained by a procedure analogous to the procedure described in Example 1 (the intermediate was replaced to 4,6-dichloro-[4,5-c]pyridine-7-carboxamide) was a light yellow solid. ¹H NMR (400 MHz, CD3OD) δ: 7.41-7.04 (m, 9H), 6.27-6.17 (m, 1H), 6.13-6.03 (m, 1H), 5.59 (dd, 1H), 4.01 (br s, 1H), 3.39 (br s, 1H), 3.17 (br s, 1H), 2.64 (br s, 1H), 2.43 (br s, 1H), 1.99-1.68 (m, 3H), 1.35 (m, 1H); LC/MS(ESI): m/z=499.2 [M+H]⁺.

### Example 4

### (S)-4-(3-acrylamidopyrrolidin-1-yl)-2-oxo-6-(4-phenoxyphenyl)-2,3-dihydro-1H-imidazo[4,5-c]pyridine-7-carboxamide (Compound 4)

The compound 2 (55 mg, yield 38%, which was the yield of the final step, the same below) obtained by a procedure analogous to the procedure described in Example 1 (the intermediate was replaced to 4,6-dichloro-[4,5-c]pyridine-7-carboxamide) was a light yellow solid. ¹H NMR (400 MHz, CD3OD) δ: 7.42-7.04 (m, 9H), 6.71-6.54 (d, 1H), 6.32-6.22 (m, 1H), 5.74 (m, 1H),4.40-4.23 (m, 2H), 3.91 (dd, 1H), 3.86-3.58 (m, 3H), 2.24-2.02 (m, 2H); LC/MS(ESI): m/z=495.2[M+H]⁺.

### Example 5

### (S)-4-(3-(but-2-ynamido)pyrrolidin-1-yl)-2-oxo-6-(4-phenoxyphenyl)-2,3-dihydro-1H-imidazo[4,5-c]pyridine-7-carboxamide (Compound 5)

The compound 5 (52 mg, yield 35%, which was the yield of the final step, the same below) obtained by a procedure analogous to the procedure described in Example 1 (the intermediate was replaced to 4,6-dichloro-[4,5-c]pyridine-7-carboxamide) was a light yellow solid. LC/MS(ESI): m/z=497[M+H]⁺.

### Example 6

### (R)-4-(3-(but-2-ynamido)piperidin-1-yl)-2-oxo-6-(4-phenoxyphenyl)-2,3-dihydro-1H-imidazo[4,5-c]pyridine-7-carboxamide (Compound 6)

The compound (60 mg, 39%) was a light yellow solid. ¹H NMR (400 MHz, CD3OD) δ: 7.42-7.04 (m, 9H), 3.98-3.83 (m, 1H), 3.19-3.07 (m, 1H), 3.06-2.89 (m, 1H), 3.06-2.91 (m, 1H), 2.80 (br s, 1H), 1.93 (s, 3H), 1.91-1.80 (m, 1H), 1.73 (s, 2H), and 1.35 (s, 1H); LC/MS(ESI): m/z=511.2[M+H]⁺.

### Example 7

### (R)-4-(3-(but-2-ynamido)piperidin-1-yl)-6-(4-phenoxyphenyl)-1H-pyrazolo [4,3-c]pyridine-7-carboxamide (Compound 7)

The compound 7 (54 mg, yield 36%, which was the yield of the final step, the same below) obtained by a procedure analogous to the procedure described in Example 1 (the intermediate was replaced to 4,6-dichloro-[4,5-c]pyridine-7-carboxamide) was a light yellow solid. ¹H NMR (400 MHz, CD3OD) δ: 7.41-7.04 (m, 9H), 6.27-6.17 (m, 1H), 6.13-6.03 (m, 1H), 5.59 (dd, 1H), 4.01 (br s, 1H), 3.39 (br s, 1H), 3.17 (br s, 1H), 2.64 (br s, 1H), 2.43 (br s, 1H), 1.99-1.68 (m, 3H), 1.35 (s, 1H); LC/MS(ESI): m/z=499[M+H]⁺.

### Example 8

### (R)-4-(3-acrylamidopiperidin-1-yl)-2-oxo-6-(4-phenoxyphenyl)-2,3-dihydro-1H-imidazo[4,5-c]pyridine-7-carboxamide (Compound 8)

The compound 2 (45 mg, yield 31%, which was the yield of the final step, the same below) obtained by a procedure analogous to the procedure described in Example 1 (the intermediate was replaced to 4,6-dichloro-[4,5-c]pyridine-7-carboxamide) was a light yellow solid. ¹H NMR (400 MHz, CD3OD) δ: 7.56-7.04 (m, 9H), 6.71-6.54 (d, 1H), 6.32-6.22 (m, 1H), 5.74 (m, 1H), 4.40-4.23 (m, 2H), 3.91 (dd, 1H), 3.86-3.58 (m, 3H), 2.24-2.02 (s, 2H); LC/MS(ESI): m/z=485 [M+H]⁺.

### Example 9

### (R)-4-(3-(but-2-ynamido)pyrrolidin-1-yl)-2-oxo-6-(4-phenoxyphenyl)-2,3-dihydro-1H-imidazo[4,5-c]pyridine-7-carboxamide (Compound 9)

The compound 2 (55 mg, yield 37%, which was the yield of the final step, the same below) obtained by a procedure analogous to the procedure described in Example 1 (the intermediate was replaced to 4,6-dichloro-[4,5-c]pyridine-7-carboxamide) was a light yellow. LC/MS(ESI): m/z=497[M+H]⁺.

### Example 10

### (S)-5-(3-(but-2-ynamido)piperidin-1-yl)-2,3-dioxo-7-(4-phenoxyphenyl)-1,2,3,4-tetrahydropyrido[3,4-b]pyrazine-8-carboxamide (Compound 10)

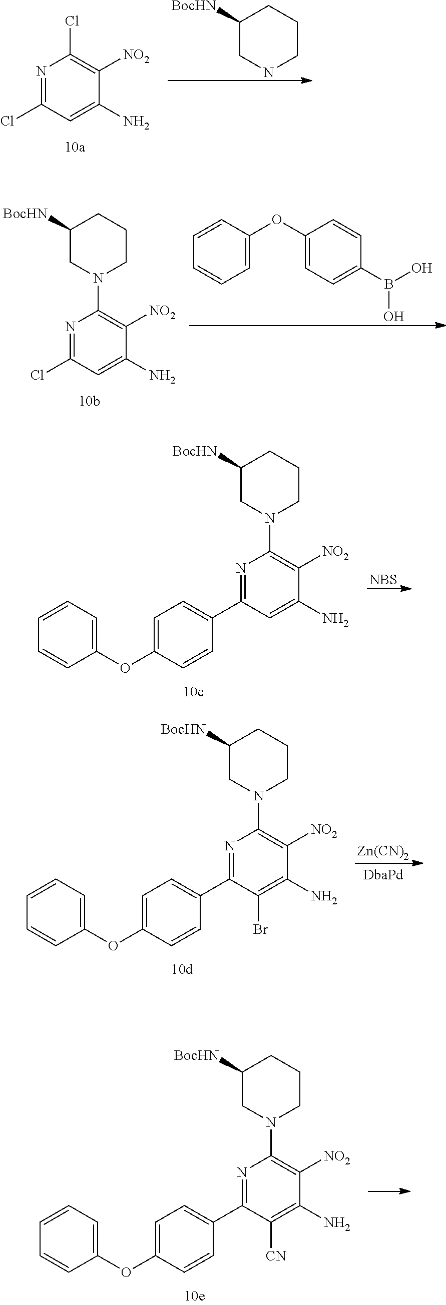

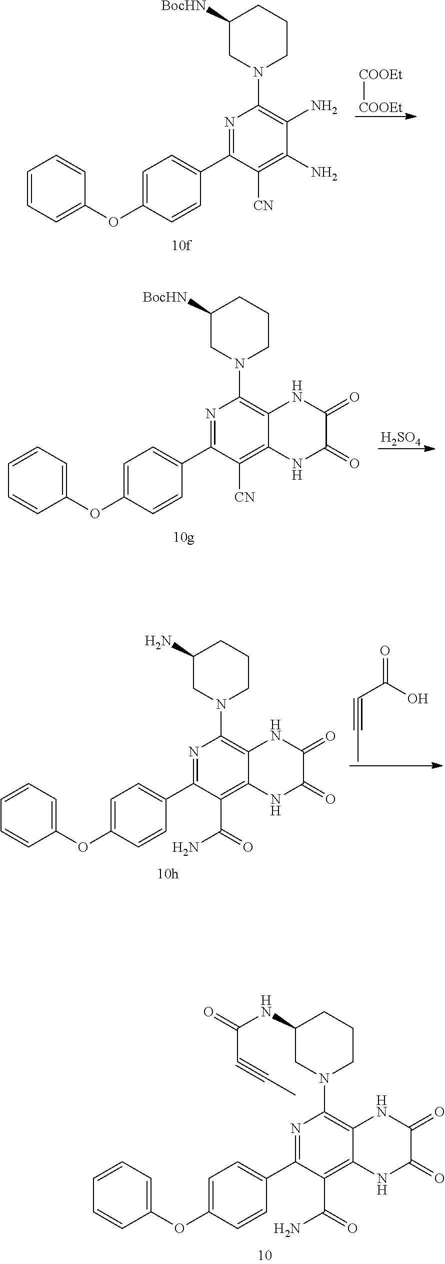

### Step 1: Synthesis of Compound 10b

The 2,6-dichloro-3-nitropyridin-4-amine 10a (20.8 g, 0.1 mol), (S)-tert-butyl piperidin-3-ylcarbamate (22 g, 0.11 mol), potassium carbonate (22 g, 0.2 mmol) catalytic amount KI and DMF (2000 mL) were mixed. The mixture was heated to 120° C. and stirred for 4 h. After cooling to room temperature, the mixture was depressurized and subjected to solvent evaporation, so as to obtain a yellow solid (20.8 g, 56%). LC/MS(ESI): m/z=373[M+H]⁺.

### Step 2: Synthesis of Compound 10c

The product 10b (18.59 g, 0.05 mol) obtained in the previous step, 4-phenoxyphenylboronic acid (10.7 g, 0.05 mol), tris(dibenzylideneacetone)dipalladium (4 g, 4.4 mmol), cesium carbonate, 1,4-dioxane (500 mL) and water (100 mL) were mixed, and then the mixture was refluxed and heated to 120° C., stirred and reacted for 16 h. The reactant was cooled to the room temperature and stirred overnight to produce a yellowish precipitate. The reaction mixture was diluted with water (10 mL), and the solid was collected through filtration. The crude product was pulped with methanol (50 mL), so as to obtain an off-white solid (12.9 g, 51%), and purification was not required for the next reaction. LC/MS(ESI): m/z=506[M+H]⁺.

### Step 3: Synthesis of Compound 10d

The NBS (5.2 g, 29.6 mmol) was added to the solution of 10c (12.1 g, 24 mmol) in acetic acid (100 mL), and the acetic acid was removed under pressure relief after the mixture was heated to 60° C. and stirred for 2 h. The residue was suspended in water (60 mL), and the saturated sodium bicarbonate solution (40 mL) was added. The solid was filtered and stirred for 30 min in 80° C. water (200 mL). After cooling to room temperature, the solid was filtered and dried in vacuum, so as to obtain a crude product yellowish-brown solid 10d (12.2 g, 87%), and purification was not required for the next reaction, LC/MS(ESI): m/z=585[M+H]⁺.

### Step 4: Synthesis of Compound 10e

The mixture of 10d (7.8 g, 13.35 mmol), Zn(CN)₂ (940 mg, 8 mmol), tris(dibenzylideneacetone)dipalladium (0.61 g, 0.65 mmol) and 1,1′-Bis(diphenylphosphino)ferrocene (0.74 g, 1.35 mmol) was added to DMF/H2O (99.1, 50 mL) under nitrogen atmosphere, and the mixture was stirred for 30 min, then heated to 120° C. and stirred for 24 h. After cooling to room temperature, the obtained mixture was precipitated with the saturated NH₄Cl solution:stronger ammonia water:H₂O (4:1:4, 10 mL). The reactant was cooled to 0° C. and filtered, the filter cake was washed by the saturated NH₄Cl solution:stronger ammonia water:H₂O (4:1:4, 2 mL), after drying in vacuum, the dark brown solid (5.38 g, 76%) was obtained. Purification was not required for the next reaction, LC/MS(ESI): m/z=531[M+H]⁺.

### Step 5: Synthesis of Compound 10f

10e (5.31 g, 10 mmol) was dissolved in 50 mL of ethyl alcohol, and hydrogenated for 4 h with raney Ni (2.0 g) in the room temperature and under 1.0 atm H₂ atmosphere. After the reaction and adding 1.6 g kieselguhr was added to the solution, the mixture was sharply stirred and filtered on a kieselguhr pad. The filtrate was purified with silica gel column chromatography, so as to obtain a dark brown solid 10f (4.70 g, 94%). Purification was not required for the next reaction, LC/MS(ESI): m/z=501[M+H]⁺.

### Step 6: Synthesis of Compound 10g

Sodium methoxide (0.63 g) was added to the solution of 10f (2.5 g, 5 mmol) and 20 ml of methyl alcohol, and the mixture was stirred for 30 min at the room temperature. The solution that diethyl oxalate (0.76 g, 5.1 mmol) was dissolved in MeOH (8 mL) was added to the mixture for 30 min. The obtained mixture was heated to reflux for 7 h. The mixture was concentrated in pressure relief and cooled in a water bath. The reaction mixture was adjusted to PH=6.5 with 10% hydrochloric acid. The settled solid was collected through filtration, washed with water and dried, so as to obtain a yellow solid compound 10g (2.6 g, 94%). Purification was not required for the next reaction, LC/MS(ESI): m/z=555[M+H]⁺.

### Step 7: Synthesis of Compound 10h

The compound 10g (1.66 g, 3 mmol) in the previous step was added in 80% of sulfuric acid (11 mL) in batches, stirred and reacted for 2.5 h at 60° C. After cooling to room temperature, the reaction mixture was added to ice water and heated to the room stirring temperature. PH was adjusted to 8 with KOH, extracted with ethyl acetate (2×). The mixture was dried with anhydrous sodium sulfate, and concentration and decompression, the brown solid intermediate 10h (1.40 g, 97%) was obtained. Purification was not required for the next reaction, LC/MS(ESI): m/z=473[M+H]⁺.

### Step 8: Synthesis of Compound 10

The intermediate 10h (237 mg, 0.5 mmol), N,N-dimethylformamide (8 mL), -2-butynoic acid (46.2 mg, 0.55 mmol), HATU (379 mg, 164 mmol) and DIPEA (275 μL) were added to 25 mL of three-necked flask (the temperature raised to 35° C.). The final solution was that it was stirred for 2 h at the room temperature, the mixture ethyl acetate (10 mL) and water washing (5 mL) were diluted with diluent. The organic phase separation and water layer were extracted (2×10 ml) with ethyl acetate. The combined organic extract was washed by washing liquid (including less sodium chloride), washed with saline (10 ml), and dried with anhydrous sodium sulfate. The crude product was obtained under decompression, and through column chromatography purification, the yellow solid compound 10 (140 mg, yield 52%) was obtained. ¹H NMR (400 MHz, CD3OD) δ: 7.41-7.04 (m, 11H), 3.98-3.83 (m, 1H), 3.19-3.07 (m, 1H), 3.06-2.89 (m, 1H), 3.06-2.91 (m, 1H), 2.80 (br s, 1H), 1.96 (s, 3H), 1.91-1.80 (m, 1H), 1.73 (s, 2H) and 1.35 (s, 1H); LC/MS(ESI): m/z=539.2[M+H]⁺.

### Example 11

### (R)-5-(3-(but-2-ynamido)piperidin-1-yl)-2,3-dioxo-7-(4-phenoxyphenyl)-1,2,3,4-tetrahydropyrido[3,4-b]pyrazine-8-carboxamide (Compound 11)

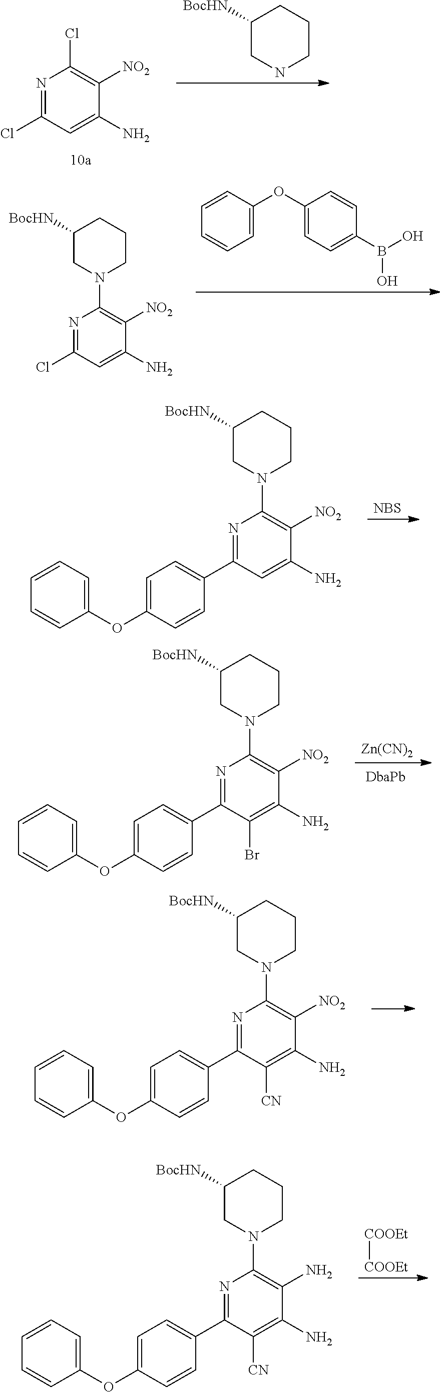

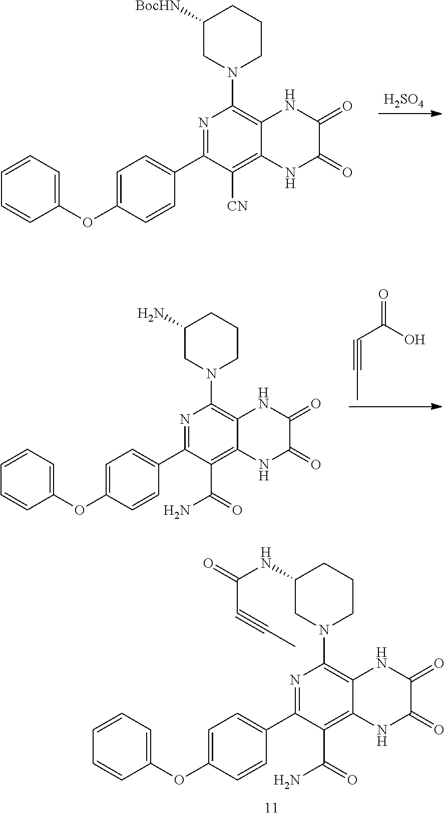

The compound 11 (129 mg, yield 48%, which was the yield of the final step, the same below) obtained by a procedure analogous to the procedure described in Example 10 (the intermediate was replaced to ((S)-tert-butyl piperidin-3-ylcarbamate) was a light yellow solid. ¹H NMR (400 MHz, CD3OD) δ: 7.41-7.04 (m, 9H), 3.98-3.84 (m, 1H), 3.19-3.06 (m, 1H), 3.06-2.89 (m, 1H), 3.08-2.90 (m, 1H), 2.81 (br s, 1H), 1.97 (s, 3H), 1.91-1.81 (m, 1H), 1.73 (s, 2H) and 1.36 (s, 1H); LC/MS(ESI): m/z=539.2[M+H]⁺.

### Example 12

### (S)-5-(3-acrylamidopiperidin-1-yl)-2,3-dioxo-7-(4-phenoxyphenyl)-1,2,3,4-tetrahydropyrido[3,4-b]pyrazine-8-carboxamide (Compound 12)

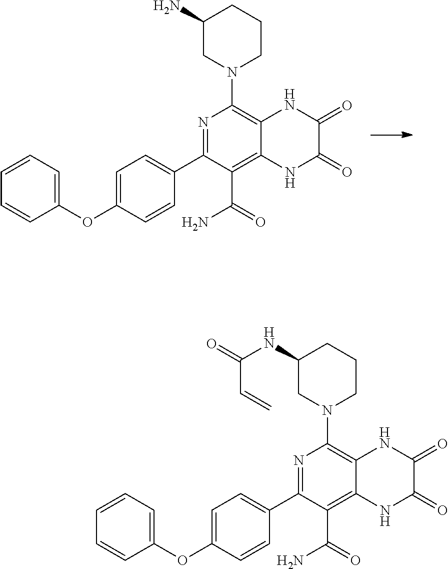 [SMILES: NC(=O)c1c(-c2ccc(Oc3ccccc3)cc2)nc(N2CCC[C@H](N)C2)c2[nH]c(=O)c(=O)[nH]c12 ; C=CC(=O)N[C@H]1CCCN(c2nc(-c3ccc(Oc4ccccc4)cc3)c(C(N)=O)c3[nH]c(=O)c(=O)[nH]c23)C1]

The compound 12 (100 mg, yield 38%, which was the yield of the final step, the same below) obtained by a procedure analogous to the procedure described in Example 10 (the intermediate was replaced to acrylic acid) was a light yellow solid. ¹H NMR (400 MHz, CD3OD) δ: 7.43-7.04 (m, 9H), 6.27-6.17 (m, 1H), 6.13-6.03 (m, 1H), 5.59 (dd, 1H), 4.01 (br s, 1H), 3.39 (br s, 1H), 3.17 (br s, 1H), 2.64 (br s, 1H), 2.43 (br s, 1H), 1.99-1.68 (m, 3H), 1.35 (m, 1H); LC/MS(ESI): m/z=527.2[M+H]⁺.

### Example 13

### (R)-5-(3-acrylamidopiperidin-1-yl)-2,3-dioxo-7-(4-phenoxyphenyl)-1,2,3,4-tetrahydropyrido[3,4-b]pyrazine-8-carboxamide (Compound 13)

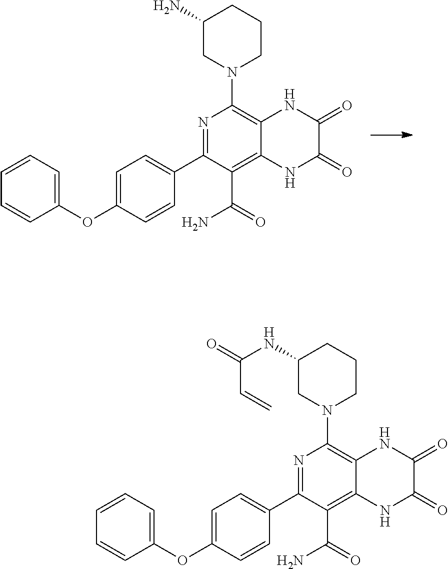 [SMILES: NC(=O)c1c(-c2ccc(Oc3ccccc3)cc2)nc(N2CCC[C@@H](N)C2)c2[nH]c(=O)c(=O)[nH]c12 ; C=CC(=O)N[C@@H]1CCCN(c2nc(-c3ccc(Oc4ccccc4)cc3)c(C(N)=O)c3[nH]c(=O)c(=O)[nH]c23)C1]

The compound 13 (84 mg, yield 32%, which was the yield of the final step, the same below) obtained by a procedure analogous to the procedure described in Example 10 (the intermediate was replaced to acrylic acid) was a light yellow solid. ¹H NMR (400 MHz, CD3OD) δ: 7.43-7.04 (m, 9H), 6.27-6.17 (m, 1H), 6.13-6.03 (m, 1H), 5.59 (dd, 1H), 4.01 (br s, 1H), 3.39 (br s, 1H), 3.17 (br s, 1H), 2.64 (br s, 1H), 2.43 (br s, 1H), 1.99-1.68 (m, 3H), 1.35 (m, 1H); LC/MS(ESI): m/z=511.2[M+H]⁺.

### Example 14

### (S)-5-(3-(but-2-ynamido)pyrrolidin-1-yl)-2,3-dioxo-7-(4-phenoxyphenyl)-1,2,3,4-tetrahydropyrido[3,4-b]pyrazine-8-carboxamide (Compound 14)

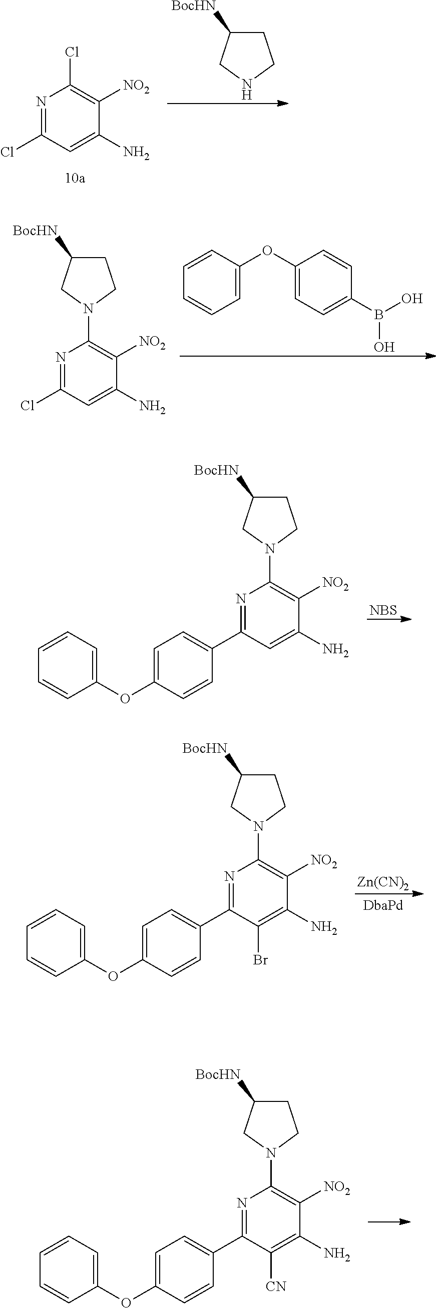

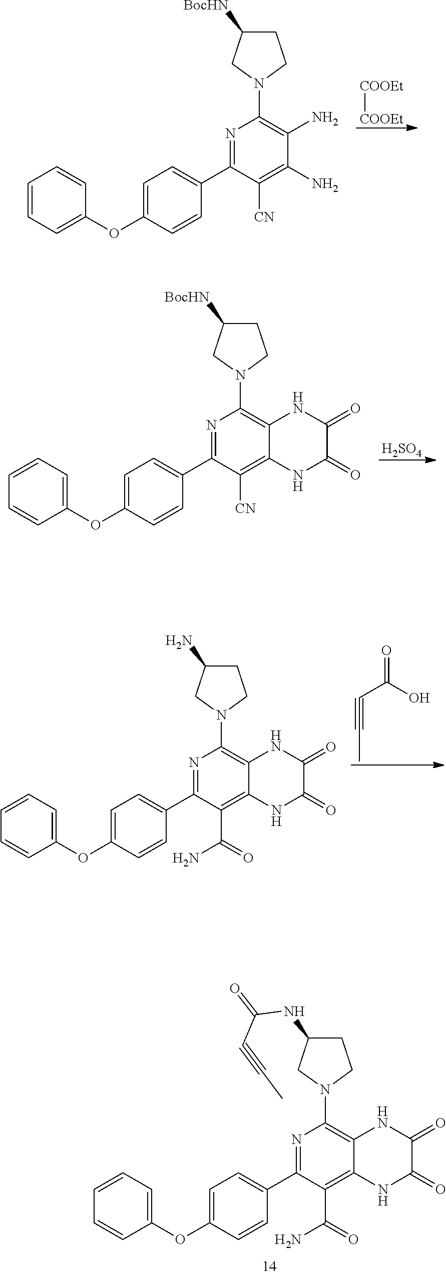

The compound 14 (147 mg, yield 56%, which was the yield of the final step, the same below) obtained by a procedure analogous to the procedure described in Example 10 (the intermediate was replaced to (S)-tert-butyl pyrrolidin-3-yl carbamate) was a light yellow solid. ¹H NMR (400 MHz, CD3OD) δ: 7.42-7.06 (m, 9H), 4.34 (m, 1H), 3.26-3.13 (m, 2H), 3.11-2.97 (m, 2H), 2.24 (m, 1H), 1.94 (s, 3H), 1.85 (m, 1H); LC/MS(ESI): m/z=525.2[M+H]⁺.

### Example 15

### (R)-5-(3-(but-2-ynamido)pyrrolidin-1-yl)-2,3-dioxo-7-(4-phenoxyphenyl)-1,2,3,4-tetrahydropyrido[3,4-b]pyrazine-8-carboxamide (Compound 15)

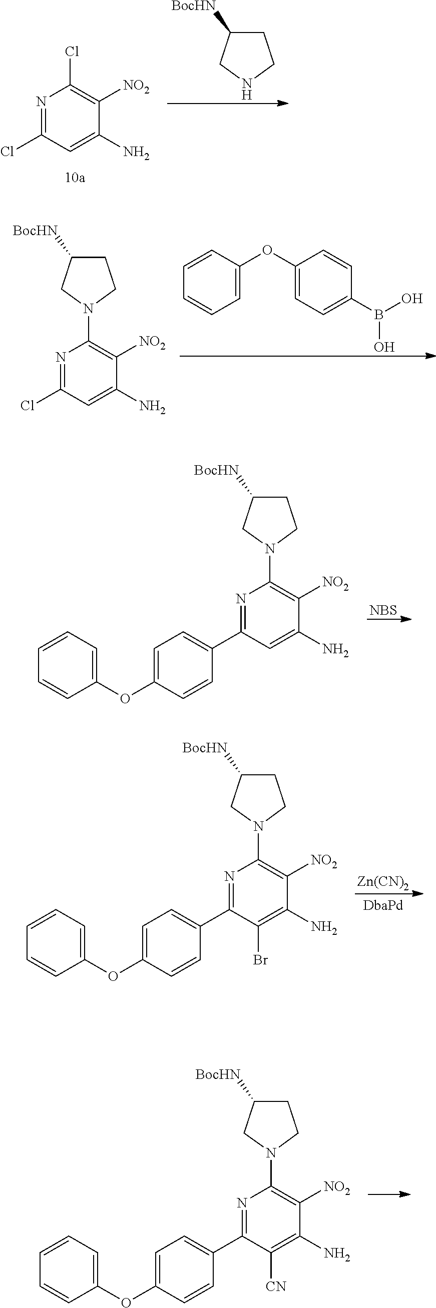

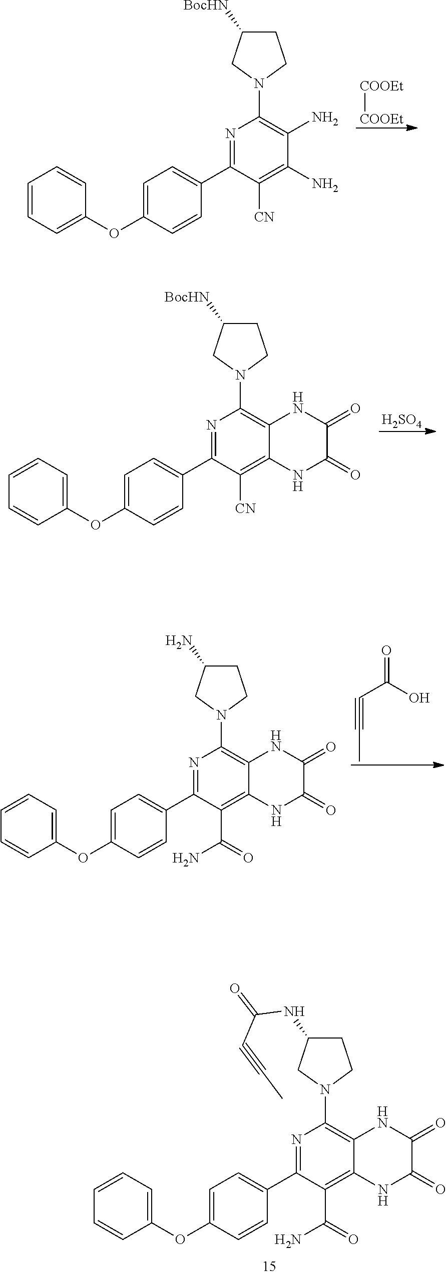

The compound 15 (160 mg, yield 61%, which was the yield of the final step, the same below) obtained by a procedure analogous to the procedure described in Example 10 (the intermediate was replaced to (R)-3-Boc-aminopyrrolidine) was a light yellow solid. ¹H NMR (400 MHz, CD3OD) δ: 7.42-7.06 (m, 9H), 4.34 (m, 1H), 3.26-3.13 (m, 2H), 3.11-2.97 (m, 2H), 2.24 (m, 1H), 1.94 (s, 3H), 1.85 (m, 1H); LC/MS(ESI): m/z=525.2[M+H]⁺.

### Example 16

### (S)-5-(3-acrylamidopyrrolidin-1-yl)-2,3-dioxo-7-(4-phenoxyphenyl)-1,2,3,4-tetrahydropyrido[3,4-b]pyrazine-8-carboxamide (Compound 16)

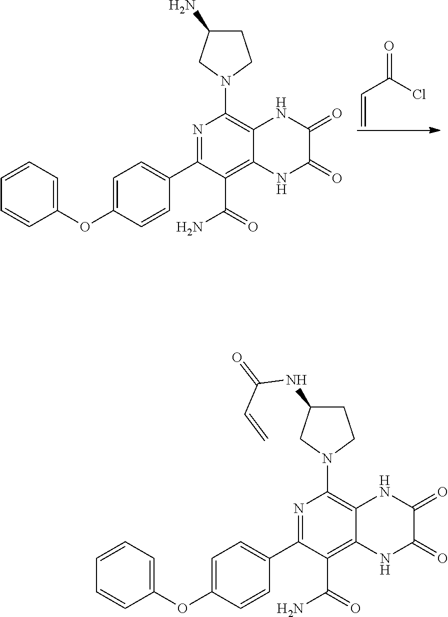

The compound 16 (97 mg, yield 38%, which was the yield of the final step, the same below) obtained by a procedure analogous to the procedure described in Example 10 (the intermediate was replaced to (S)-3-Boc-aminopyrrolidine) was a light yellow solid. ¹H NMR (400 MHz, CD3OD) δ: 7.42-7.06 (m, 9H), 6.71-6.55 (d, 1H), 6.32-6.22 (m, 1H), 5.74 (m, 1H), 4.40-4.23 (m, 2H), 3.91 (dd, 1H), 3.86-3.58 (m, 3H), 2.24-2.02 (s, 2H); LC/MS(ESI): m/z=513.2[M+H]⁺.

### Example 17

### (R)-5-(3-acrylamidopyrrolidin-1-yl)-2,3-dioxo-7-(4-phenoxyphenyl)-1,2,3,4-tetrahydropyrido[3,4-b]pyrazine-8-carboxamide (Compound 17)

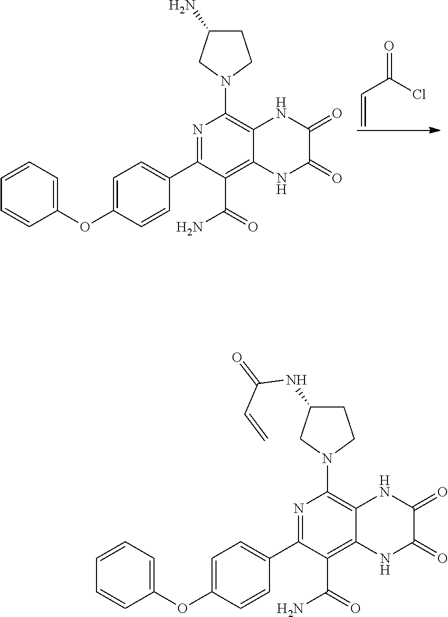

The compound 17 (92 mg, yield 36%, which was the yield of the final step, the same below) obtained by a procedure analogous to the procedure described in Example 10 (the intermediate was replaced to (R)-3-Boc-aminopyrrolidine) was a light yellow solid. ¹H NMR (400 MHz, CD3OD) δ: 7.42-7.06 (m, 9H), 6.71-6.55 (d, 1H), 6.32-6.22 (m, 1H), 5.74 (m, 1H), 4.40-4.23 (m, 2H), 3.91 (dd, 1H), 3.86-3.58 (m, 3H), 2.24-2.02 (s, 2H); LC/MS(ESI): m/z=513.2[M+H]⁺.

### Example 18

### (S)-4-(3-(but-2-ynamido)piperidin-1-yl)-6-(4-phenoxyphenyl)-1H-imidazo[4,5-c]pyridine-7-carboxamide (Compound 18)

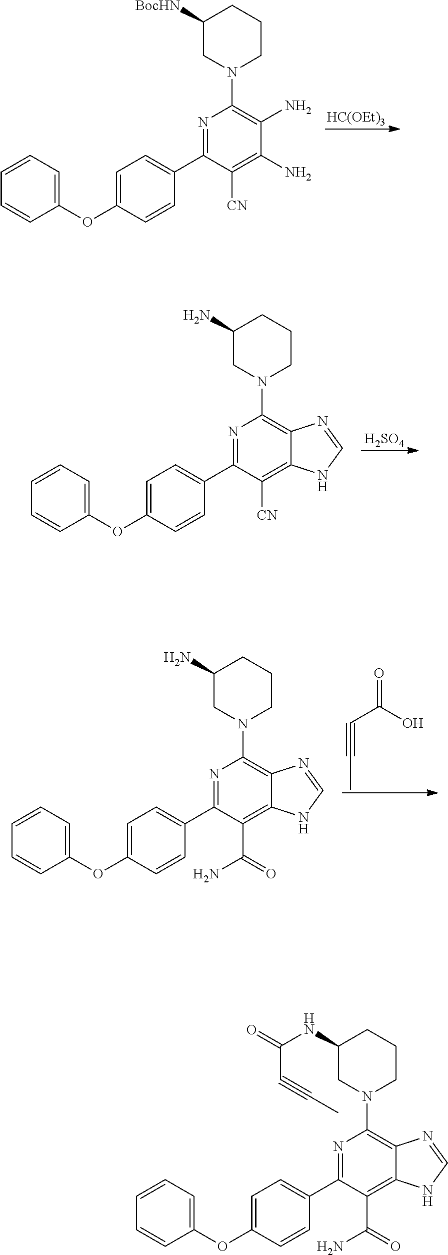

The compound 18 (123 mg, yield 52%, which was the yield of the final step, the same below) obtained by a procedure analogous to the procedure described in Example 10 (the intermediate was replaced to triethyl orthoformate) was a light yellow solid. ¹H NMR (400 MHz, CD3OD) δ: 8.13 (s, 1H), 7.41-7.06 (m, 9H), 3.98-3.83 (m, 1H), 3.19-3.07 (m, 3H), 3.06-2.89 (m, 1H), 3.06-2.91 (m, 1H), 2.80 (br s, 1H), 1.96 (s, 3H), 1.91-1.80 (m, 1H), 1.73 (m, 2H), 1.35 (m, 1H); LC/MS(ESI): m/z=495.2[M+H]⁺.

### Example 19

### (S)-4-(3-(but-2-ynamido)piperidin-1-yl)-2-methyl-6-(4-phenoxyphenyl)-1H-imidazo[4,5-c]pyridine-7-carboxamide (Compound 19)

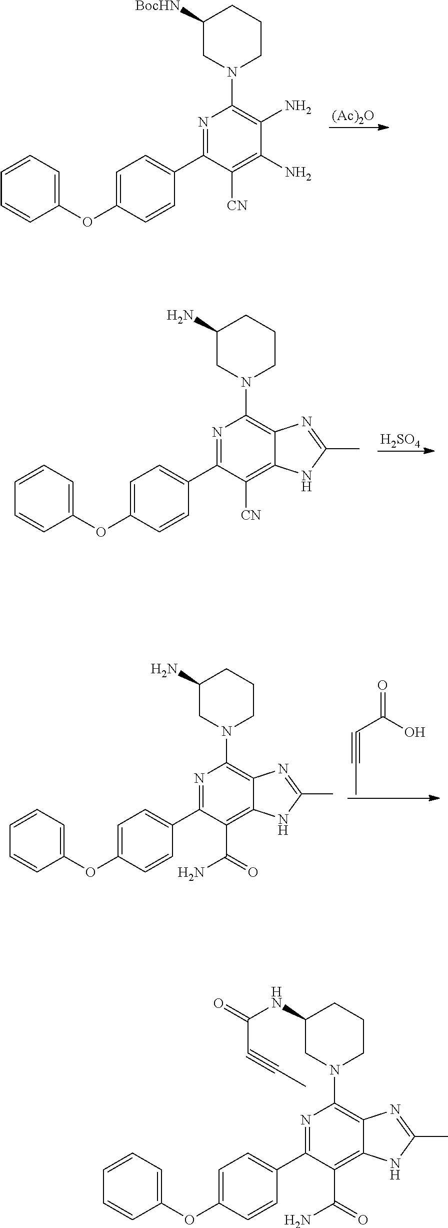

The compound 19 (124 mg, yield 49%, which was the yield of the final step, the same below) obtained by a procedure analogous to the procedure described in Example 1 (the intermediate was replaced to acetic anhydride) was a light yellow solid. ¹H NMR (400 MHz, CD3OD) δ: 7.43-7.08 (m, 9H), 3.98-3.83 (m, 1H), 3.19-3.07 (m, 1H), 3.06-2.89 (m, 1H), 3.06-2.91 (m, 1H), 2.80 (br s, 1H), 2.54 (s, 3H), 1.96 (s, 3H), 1.91-1.80 (m, 1H), 1.73 (m, 2H), 1.35 (m, 1H); LC/MS(ESI): m/z=509.2[M+H]⁺.

### Example 20

### 4-(1-acryloylpiperidin-4-yl)-2-oxo-6-(4-phenoxyphenyl)-2,3-dihydro-1H-imidazo[4,5-c]pyridine-7-carboxamide (Compound 20)

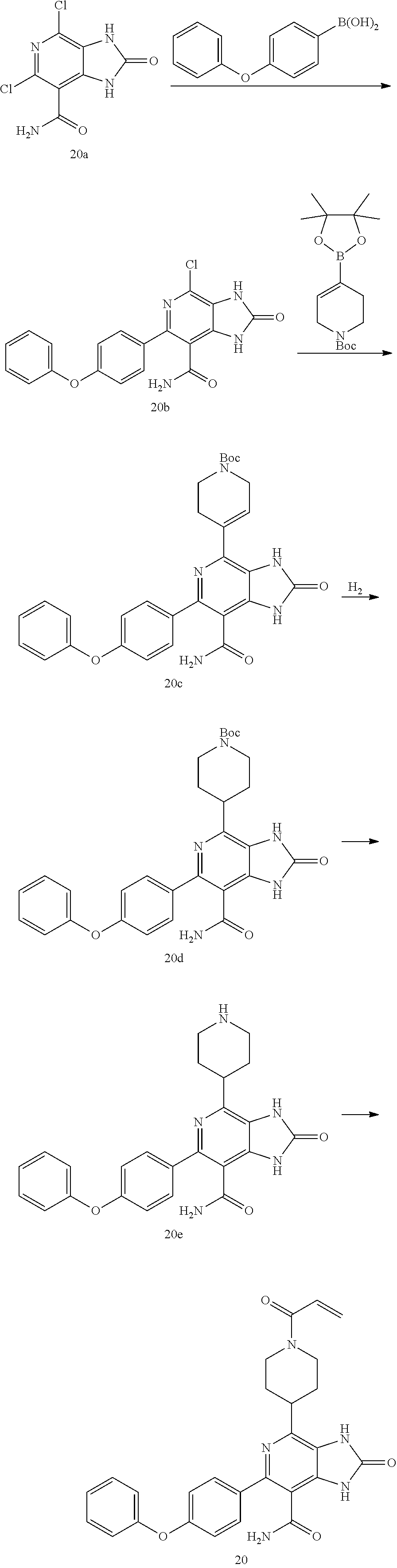

Step 1: Synthesis of 6-(4-[henoxyphenyl-2-oxo-imidazole[4,5-c]pyridine-8-formamide 20a

The raw materials 4,6-dichloro-2-oxo-imidazole[4,5-c]pyridine-7-formamide 20a (3.7 g, 15 mmol), 4-phenoxyphenylboronic acid (6.42 g, 30 mmol) and tripotassium phosphate monohydrate (10.35 g, 45 mmol) were dissolved in in dioxane (200 mL) and water (20 mL). After nitrogen charging for several times, tetrakis (triphenylphosphine) palladium (2.31 g, 2 mmol) was added. The mixture was sprayed with nitrogen for another 5 minutes, then refluxed and heated for 24 hours. The reactant was cooled to room temperature and stirred overnight to obtain a yellowish precipitate. The reaction mixture was diluted with water (10 mL) and the solid was collected by filtration. The crude product was pulped with methanol (150 mL), and then an off-white solid 20b (2.91 g, 52%) was obtained. No further purification was required for the next reaction. LC/MS (ESI): m/z=382 [M+H]⁺.

### Step 2: Synthesis of 20c

The previous intermediates: 6-(4-[henoxyphenyl-2-oxo-imidazole[4,5-c]pyridine-8-formamid 20b (1.9 g, 5 mmol), N-Boc-1,2,5,6-tetrahydropyridine-4-boric acid pinacol ester (2.31 g, 7.5 mmol), potassium carbonate (2.07 g, 15 mmol), tetrakis (triphenylphosphine) palladium, 1,4-dioxane (100 mL) and water (25 mL) were mixed, heated and refluxed under nitrogen protection, and stirred for 16 hours. The reactant was cooled to room temperature and stirred overnight. The reaction solution was evaporated under reduced pressure. The an off-white solid 20c (1.0 g, 38%) was obtained by column chromatography. No further purification was required for the next reaction. LC/MS (ESI): m/z=528 [M+H]+.

### Step 3: Synthesis of 20d

The solution that the previous compound 20c (528 mg, 1 mmol) was stored in ethyl acetate (10 mL) and methanol (10 mL) was added to 10% Pd/C (0.1 g), the reactant was degased with hydrogen for 6 times, and then stirred for 12 h at room temperature under hydrogen atmosphere. The solution was filtered, the filtrate was evaporated into the crude product of brown solid for 20d (507 mg, 96%), no further purification was required for the next reaction, LC/MS (ESI): m/z=530 [M+H]+.

### Step 4: Synthesis of 20e

The previous intermediate 20d (0.265 g, 0.5 mmol), 2 ml of ethyl acetate and 4 ml of 1,4-dioxane solution of 1N HCl were added into the reaction flask. The mixture was stirred at room temperature for 2 hours, the reaction solution was neutralized with 1N sodium hydroxide solution, and extracted with ethyl acetate. The obtained organic phase was washed with saturated sodium bicarbonate and saturated salt water, dried with anhydrous sodium sulfate, and the organic phase was evaporated to dryness under reduced pressure. Compound 20e (0.170 g, yield 79%) was obtained and directly used in the next step, LC/MS (ESI): m/z=430.2 [M+H]+.

### Step 5: Synthesis of 20

The compound 20e (129 mg, 0.3 mmol), triethylamine (51 mg, 0.5 mmol), and 4 ml tetrahydrofuran were added into the reaction flask, and slowly dropped to 0.5 ml tetrahydrofuran solution of buty-2-ethynyl chloride (45 mg, 0.5 mmol) after cooling in ice water bath. After adding, the mixture was continuously stirred for 4 hours. The reaction liquid was quenched with methanol and evaporated to dryness under reduced pressure. The residue was purified by column chromatography to obtain compound 20 (52 mg, yield 36%) as a yellow solid. ¹H NMR (400 MHz, CD₃OD) δ: 7.43-7.08 (m, 9H), 3.98-3.83 (m, 1H), 3.19-3.07 (m, 1H), 3.06-2.89 (m, 1H), 3.06-2.91 (m, 1H), 2.80 (br s, 1H), 2.54 (s, 3H), 1.96 (s, 3H), 1.91-1.80 (m, 1H), 1.73 (s, 2H), and 1.35 (s, 1H); LC/MS(ESI): m/z=484.2[M+H]⁺.

### Example 21 and Example 22

### (S)-4-(1-acryloylpiperidin-3-yl)-2-oxo-6-(4-phenoxyphenyl)-2,3-dihydro-1H-imidazo[4,5-c]pyridine-8-carboxamide (Compound 21)

### (S)-4-(1-acryloylpiperidin-3-yl)-2-oxo-6-(4-phenoxyphenyl)-2,3-dihydro-1H-imidazo[4,5-c]pyridine-8-carboxamide (Compound 22)

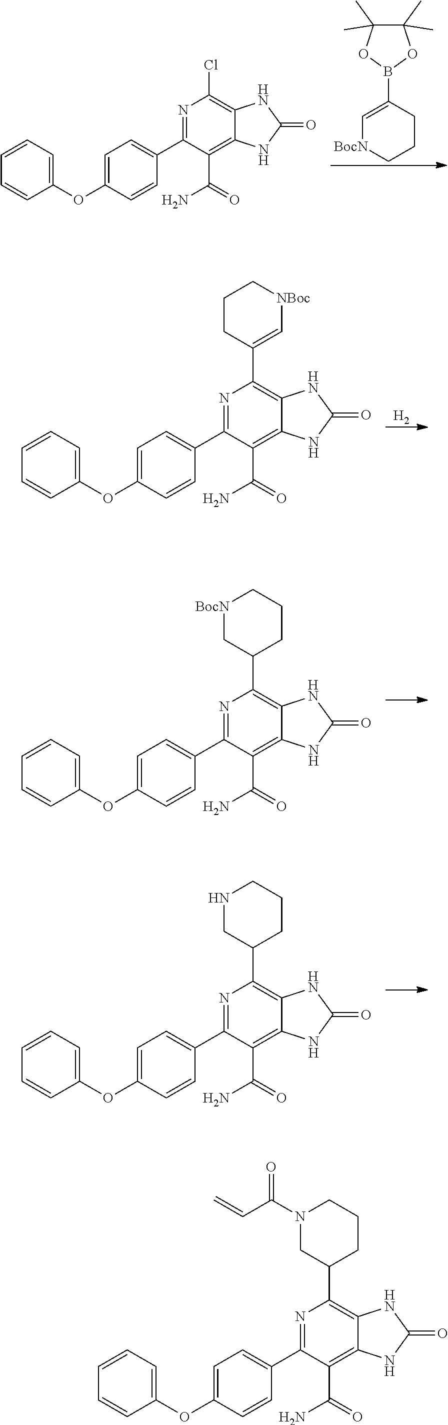

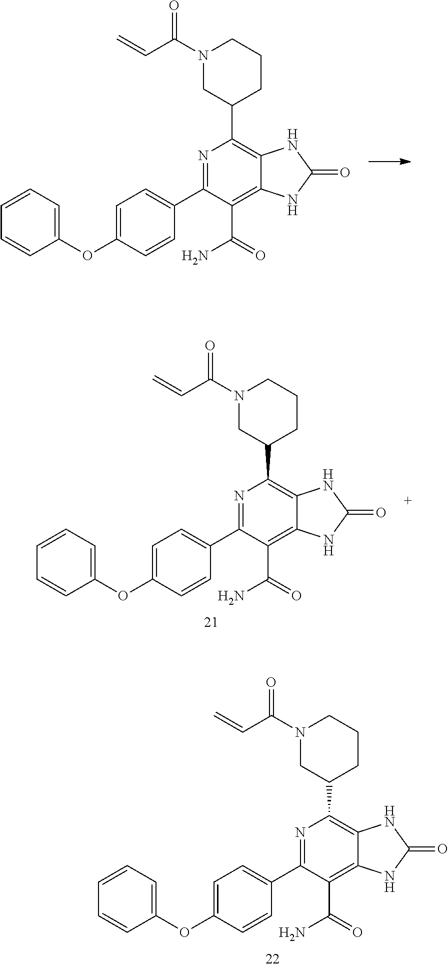

The compounds 21 and 22 (124 mg, yield 49%, which was the yield of the final step, the same below) obtained by a procedure analogous to the procedure described in Example 11 (the intermediate was replaced to 1-t-butyloxycarboryl-3,6-dihydro-2H-pyridine-5-boronic acid pinacol ester) was a light yellow solid. ¹H NMR (400 MHz, CD3OD) δ: 7.43-7.08 (m, 9H), 3.98-3.83 (m, 1H), 3.19-3.07 (m, 1H), 3.06-2.89 (m, 1H), 3.06-2.91 (m, 1H), 2.80 (br s, 1H), 2.54 (s, 3H), 1.96 (s, 3H), 1.91-1.80 (m, 1H), 1.73 (s, 2H), and 1.35 (s, 1H); LC/MS(ESI): m/z=509.2[M+H]⁺.

### Example 23

### (S)-4-(3-(but-2-ynamido)piperidin-1-yl)-6-(4-phenoxyphenyl)-1H-pyrrolo[3,2-c]pyridine-7-carboxamide (Compound 23)

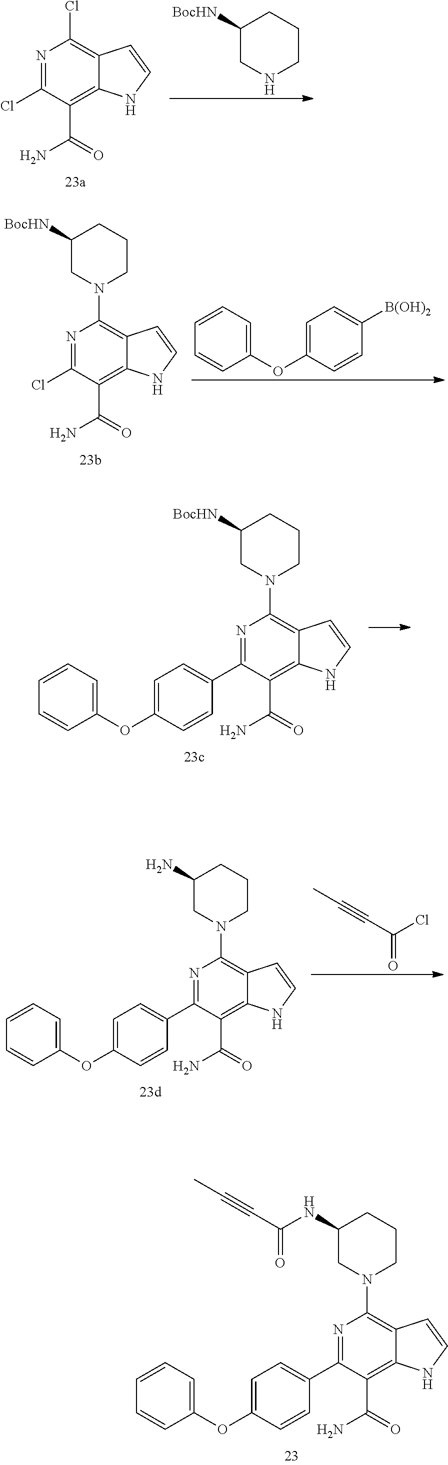

### Step 1: Synthesis of 23b

The raw material 4,6-dichloro-1H-pyrrolo[3,2-c]pyridine-7-carboxamide 23a (0.64 g, 2 mmol), (S)-3-Boc-aminopiperidine (0.44 g, 2.2 mmol), potassium carbonate (0.44 g, 4 mmol) catalytic amount of potassium iodide and DMF (40 mL) were mixed, heated to 120° C., and stirred for 4 hours. After cooling to room temperature, and evaporating under reduced pressure, yellow solid 23b (0.61 g, 78%) was obtained, LC/MS (ESI): m/z 394 [M+H]⁺.

### Step 2: Synthesis of 23c

The intermediate 23b (0.394 g, 1 mmol), 4-phenoxyphenylboronic acid (0.86 g, 4 mmol) and tripotassium phosphate monohydrate (1.38 g, 6 mmol) were dissolved in dioxane (15 mL) and water (8 mL). After multiple nitrogen charging, tetra (triphenylphosphine) palladium (0.35 g, 0.3 mmol) was added. The mixture was sprayed with nitrogen for another 5 minutes, then heated and refluxed for 24 hours. The reactant was cooled to room temperature and stirred overnight to obtain a yellowish precipitate. The reaction mixture was diluted with water (10 mL) and the solids were collected by filtration. The crude product was pulped with methanol (50 mL), and then an off-white solid 23b (0.30 g, 56%) was obtained. No further purification was required for the next reaction. LC/MS (ESI): m/z=529 [M+H]⁺.

### Step 3: Synthesis of 23d

The previous intermediate 23c (0.27 g, 0.5 mmol), 2 ml ethyl acetate and 4 ml 1,4-dioxane solution of 1N HCl were added into the reaction flask. The mixture was stirred at room temperature for 2 hours, neutralized the reaction solution with 1N sodium hydroxide solution, and extracted with ethyl acetate. The obtained organic phase was washed with saturated sodium bicarbonate and saturated salt water, dried with anhydrous sodium sulfate, and the organic phase was evaporated to dryness under reduced pressure. Compound 23d (0.187 g, yield 87%) was obtained, which was directly used in the next step, LC/MS (ESI): m/z=428.2 [M+H]⁺.

### Step 4: Synthesis of 23

The compound 23d (128 mg, 0.3 mmol), triethylamine (51 mg, 0.5 mmol), and 4 ml tetrahydrofuran were added into the reaction flask, and 0.5 ml tetrahydrofuran solution of buty-2-alkynyl chloride (45 mg, 0.5 mmol) was slowly dropped after cooling in ice water bath. After adding, it is continuously mixed for 4 hours. The reaction liquid was quenched with methanol and evaporated to dryness under reduced pressure. The residue was purified by column chromatography and compound 23 (53 mg, yield 36%) was obtained as a yellow solid. LC/MS(ESI): m/z=494.2[M+H]⁺.

### Example 24

### (R)-4-(3-(but-2-ynamido)piperidin-1-yl)-6-(4-phenoxyphenyl)-1H-pyrrolo[3,2-c]pyridine-7-carboxamide (Compound 24)

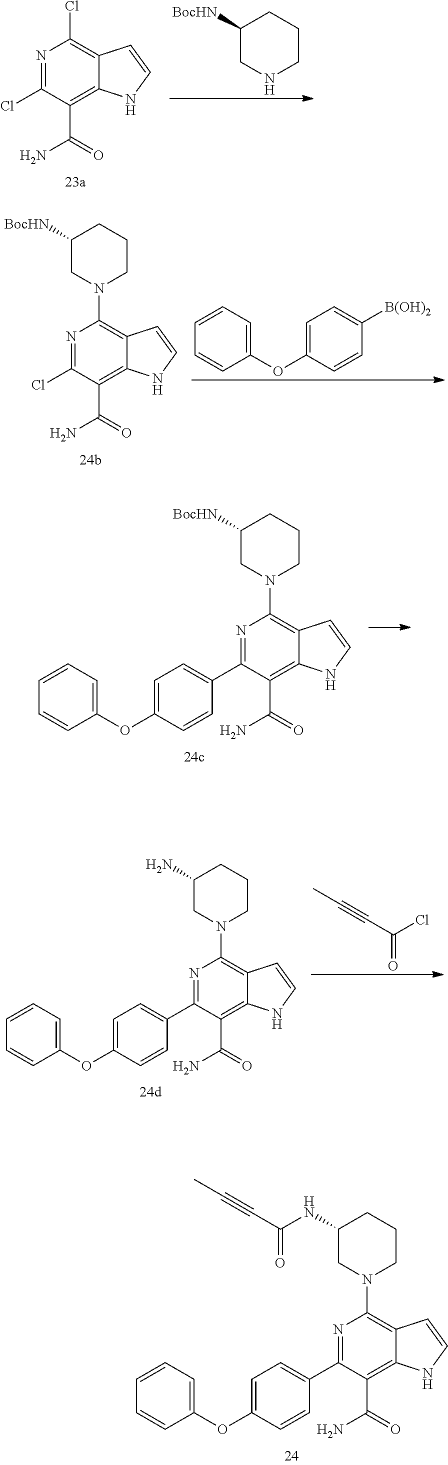

The compound 24 (68 mg, yield 46%, which was the yield of the final step, the same below) obtained by a procedure analogous to the procedure described in Example 23 (the intermediate was replaced to (R)-3-Boc-aminopiperidine) was a light yellow solid, LC/MS(ESI): m/z=494.2[M+H]⁺.

### Example 25

### 4-(1-acryloylpiperidin-4-yl)-6-(4-phenoxyphenyl)-1H-pyrrolo [3,2-c]pyridine-7-carboxamide (Compound 25)

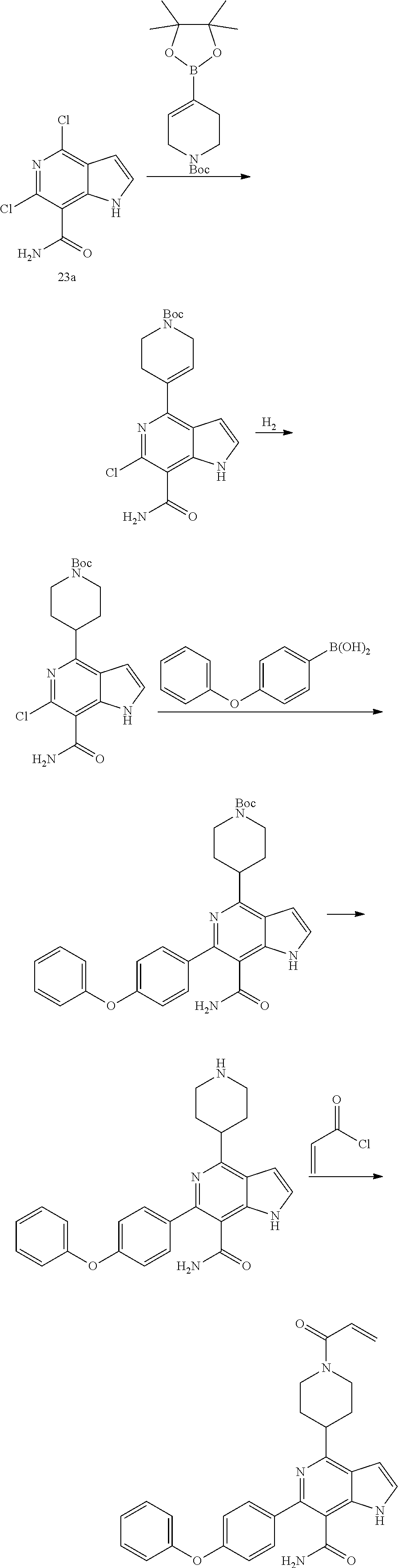

The compound 25 (59 mg, yield 42%, which was the yield of the final step, the same below) obtained by a procedure analogous to the procedure described in Example 11 (the intermediate was replaced to N-Boc-1,2,5,6-tetrahydropyridine-4-boronic acid pinacol ester) was a light yellow solid, LC/MS(ESI): m/z=467.2[M+H]⁺.

### Example 26

### (S)-4-(1-acryloylpiperidin-3-yl)-6-(4-phenoxyphenyl)-1H-pyrrolo[3,2-c]pyridine-7-carboxamide (Compound 26)

### (R)-4-(1-acryloylpiperidin-3-yl)-6-(4-phenoxyphenyl)-1H-pyrrolo[3,2-c]pyridine-7-carboxamide (Compound 27)

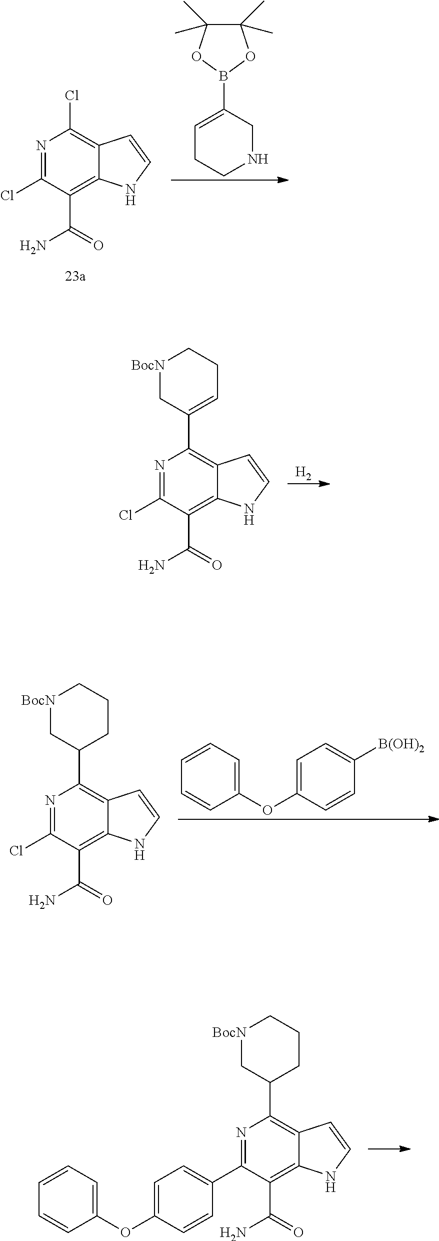

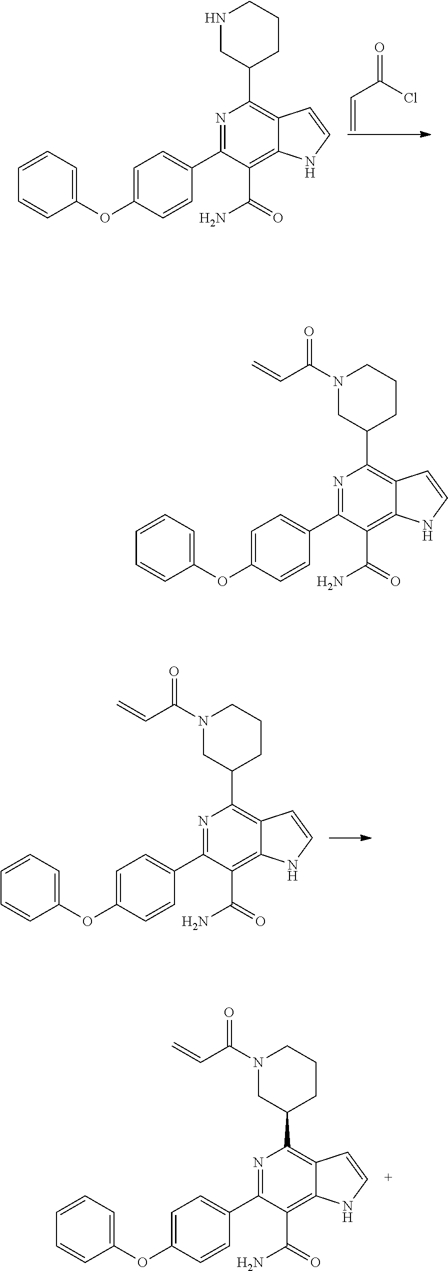

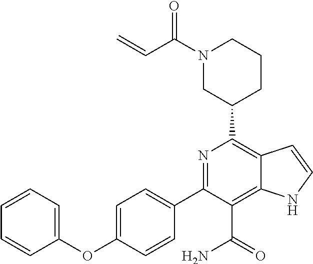 [SMILES: C=CC(=O)N1CCC[C@H](c2nc(-c3ccc(Oc4ccccc4)cc3)c(C(N)=O)c3[nH]ccc23)C1]

The racemate compound (R,S)-6-(4-phenoxyphenyl)-4-(1-acryloyl group piperidine-3-yl)-pyrrole[3,2-c)dipyridine-7-carboxamide (67 mg, yield 49%, which was the yield of the final step, the same below) obtained by a procedure analogous to the procedure described in Example 16 (the intermediate was replaced to 1-t-butyloxycarboryl-3,6-dihydro-2H-pyridine-5-boronic acid pinacol ester) was a light yellow solid, LC/MS(ESI): m/z=467.2[M+H]⁺.

### Example 28

### (S)-5-(3-(but-2-ynamido)piperidin-1-yl)-2,3-dioxo-1,2,3,4-tetrahydropyrido[3,4-b]pyrazine-8-carboxamide (Compound 28)

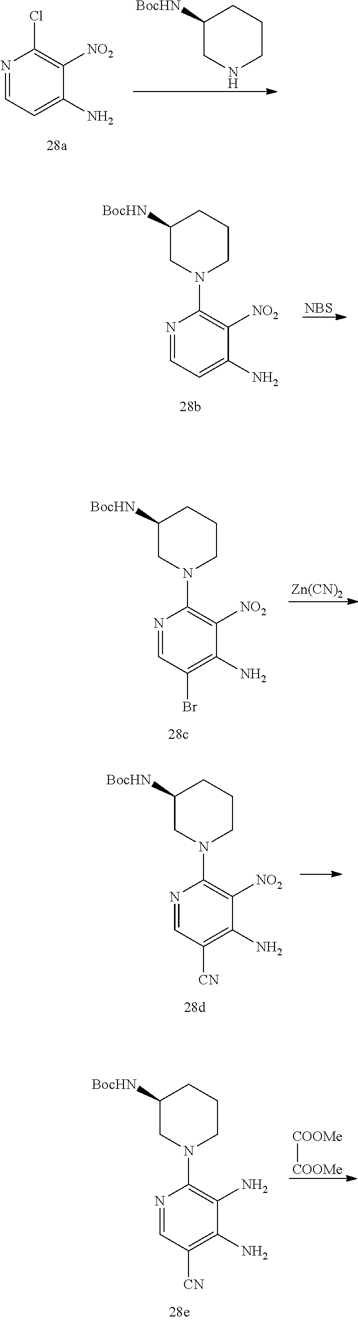

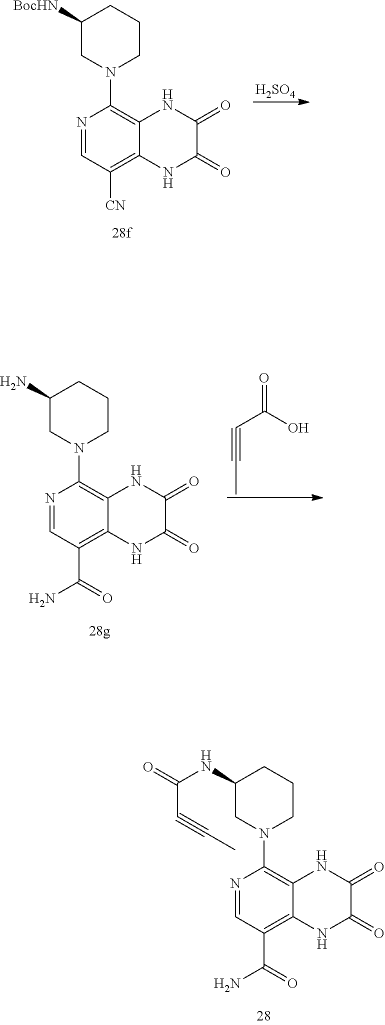

### Step 1: Synthesis of Compound 28b

The compound 28a (3.47 g, 20.0 mmol), (S)-3-t-butyloxycarboryl aminopiperidine (4.81 g, 24.0 mmol), cesium carbonate (7.82 g, 24.0 mmol) and 50 ml of N,N-dimethylformamide were added into the reaction flask. The mixture was stirred, reacted for overnight at 100° C. After cooling to room temperature, the reaction solution was diluted with ethyl acetate and water and extracted with ethyl acetate. The obtained organic phase was washed with water and saturated salt water, dried with anhydrous sodium sulfate, and evaporated to dryness under reduced pressure. The residue was purified by column chromatography to obtain the compound 28b (5.46 g, yield 81%) as a brown solid. LC/MS(ESI): m/z=238.1[M+H]⁺.

### Step 2: Synthesis of Compound 28c

The compound 28b (4.05 g, 12.0 mmol) was added into the reaction flask, 30 ml of N,N-dimethylformamide was added to NBS (3.20 g, 18.0 mmol) in batches, stirred and reacted for 24 h at 50° C. After cooling to room temperature, the reaction solution was poured to 150 ml of water and was extracted with ethyl acetate. The obtained organic phase was washed with saturated salt water, dried with anhydrous sodium sulfate, and evaporated to dryness under reduced pressure. The residue was purified by column chromatography to obtain the compound 28c (3.45 g, yield 69%) as a yellow solid. LC/MS(ESI): m/z=316.0[M+H]⁺.

### Step 3: Synthesis of Compound 28d

The compound 28c (3.33 g, 8.0 mmol), zinc cyanide (0.56 g, 4.8 mmol), 10 wt % Pd/C (0.36 g, 0.16 mmol), 1,1′-bis (diphenylphosphine) ferrocene (0.18 g, 0.32 mmol) and 15 ml of N,N-dimethylacetamide were added into the reaction flask. After replacing for 3 times with nitrogen, the zinc formate dihydrate (0.15 g, 0.8 mmol) was added, and then it was replaced for three times with nitrogen again and reacted for overnight at 100° C. After cooling to room temperature, the reaction solution was diluted with ethyl acetate and water and extracted with ethyl acetate. The obtained organic phase was washed with water and saturated salt water, dried with anhydrous sodium sulfate, and evaporated to dryness under reduced pressure. The residue was purified by column chromatography to obtain the compound 28d (2.20 g, yield 76%) as a brown solid. LC/MS(ESI): m/z=263.1[M+H]⁺.

### Step 4: Synthesis of Compound 28c

The compound 28d (3.33 g, 6.0 mmol), triacetate iron acetone (11 mg, 0.03 mmol), hydrazine hydrate (0.36 g, 7.2 mmol) and 8 ml of methyl alcohol were added into a microwave reaction tube. When stirring, the mixture was in microwave reaction for 6 h at 140° C. After cooling to room temperature, the reaction solution was evaporated to dryness under reduced pressure. The residue was purified by column chromatography to obtain the compound 28e (1.85 g, yield 93%) as a yellow solid. LC/MS(ESI): m/z=233.2[M+H]⁺.

### Step 5: Synthesis of Compound 28f

The compound 28e (1.16 g, 3.0 mmol), 2 ml of 4N hydrochloric acid and dimethyl oxalate (0.39 g, 3.3 mmol) were added into the reaction flask. When stirring, the mixture reacted for 2 h at 140° C. After cooling to room temperature, the reaction solution was evaporated to dryness under reduced pressure. The residue was purified by column chromatography to obtain the compound 28f (0.95 g, yield 82%) as a yellow solid. LC/MS(ESI): m/z=287.7[M+H]⁺.

### Step 6: Synthesis of Compound 28g

The compound 28f (0.77 g, 2.0 mmol) and 2 ml of sulfuric acid were added into the reaction flask. When stirring, the mixture reacted for 1 h at 60° C. After cooling to room temperature, the reaction solution was poured to 10 ml of ice water, neutralized with 1N of sodium hydroxide aqueous solution and extracted with ethyl acetate. The obtained organic phase was washed with saturated sodium bicarbonate and saturated salt water, dried with anhydrous sodium sulfate, and evaporated to dryness under reduced pressure. The residue was purified by column chromatography to obtain the compound 28g (0.44 g, yield 73%) as a yellow solid. LC/MS(ESI): m/z=305.1[M+H]⁺.

### Step 7: Synthesis of Compound 28

The compound 28g (304 mg, 1.0 mmol), 2 ml of N,N-dimethylformamide, 2-tetrolic acid (101 mg, 1.2 mmol), HATU (570 mg, 1.5 mmol) and N,N-diisopropylethylamine (258 mg, 2.0 mmol) were added into the reaction flask. When stirring, the mixture reacted for 2 h at the room temperature. The reaction solution was diluted with ethyl acetate and water and extracted with ethyl acetate. The obtained organic phase was washed with water and saturated salt water, dried with anhydrous sodium sulfate, and evaporated to dryness under reduced pressure. The residue was purified by column chromatography to obtain the compound 28 (230 mg, yield 62%) as a brown solid. ¹H NMR (400 MHz, CD3OD) δ: 8.5 (s, 1H), 3.99-3.83 (m, 1H), 3.19-3.06 (m, 1H), 3.06-2.89 (m, 1H), 3.08-2.89 (m, 1H), 2.81 (br s, 1H), 1.97 (s, 3H), 1.91-1.81 (s, 1H), 1.73 (s, 2H), and 1.37 (s, 1H); LC/MS(ESI): m/z=371.1[M+H]⁺.

### Example 29

### (R)-5-(3-(but-2-ynamido)piperidin-1-yl)-2,3-dioxo-1,2,3,4-tetrahydropyrido [3,4-b]pyrazine-8-carboxamide (Compound 29)

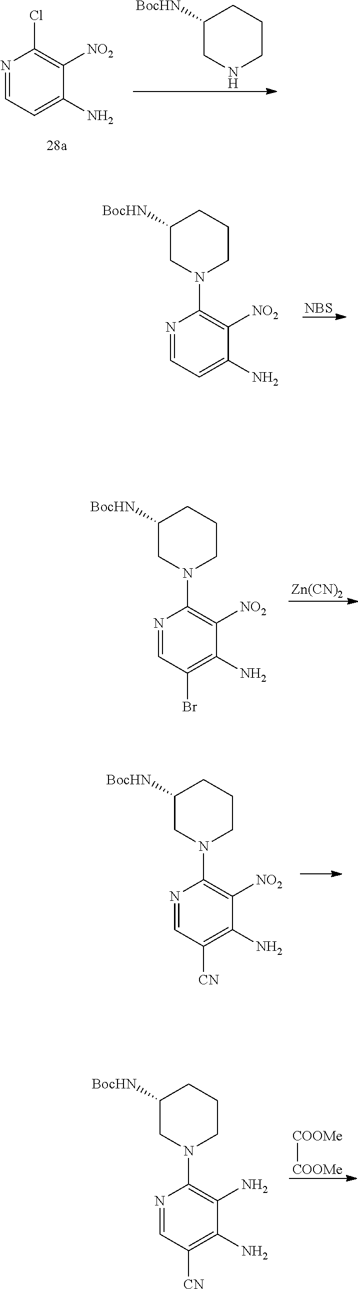

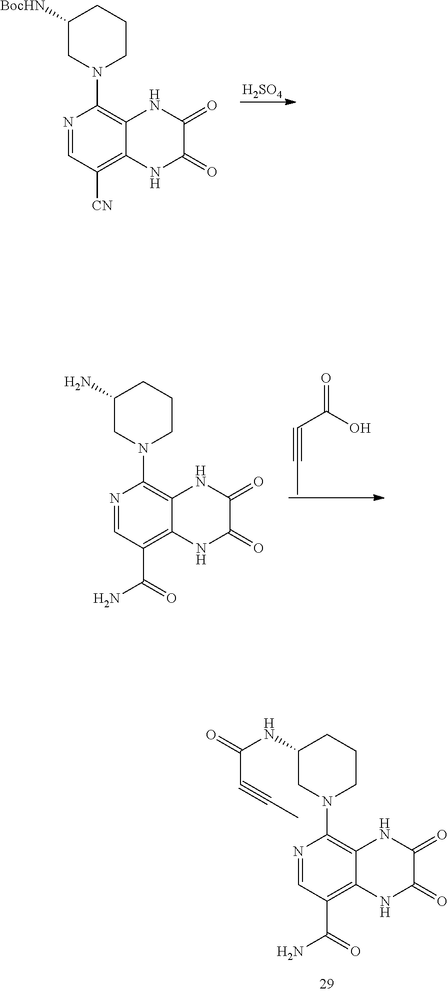

The compound 20 (215 mg, yield 58%, which was the yield of the final step, the same below) obtained by a procedure analogous to the procedure described in Example 28 (the intermediate was replaced to (R)-3-t-butyloxycarboryl aminopiperidine) was a light yellow solid. ¹H NMR (400 MHz, CD3OD) δ: 8.5 (s, 1H), 3.99-3.84 (m, 1H), 3.19-3.06 (m, 1H), 3.06-2.89 (m, 1H), 3.08-2.89 (m, 1H), 2.81 (br s, 1H), 1.97 (s, 3H), 1.91-1.81 (m, 1H), 1.73 (m, 2H), 1.37 (m, 1H); LC/MS(ESI): m/z=371.1[M+H]⁺.

### Example 30

### (S)-5-(3-acrylamidopiperidin-1-yl)-2,3-dioxo-1,2,3,4-tetrahydropyrido[3,4-b]pyrazine-8-carboxamide

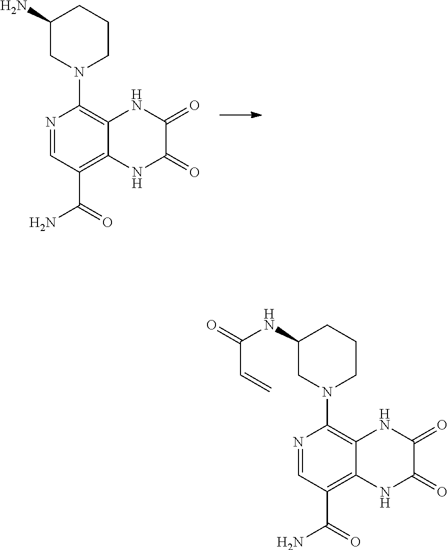 [SMILES: NC(=O)c1cnc(N2CCC[C@H](N)C2)c2[nH]c(=O)c(=O)[nH]c12 ; C=CC(=O)N[C@H]1CCCN(c2ncc(C(N)=O)c3[nH]c(=O)c(=O)[nH]c23)C1]

The compound 30 (250 mg, yield 65%) obtained by the intermediate (S)-5-(3-aminopiperidine)-2,3-dioxo-pyridine[3,4-b]quinoxaline-8-carboxamide and acrylic acid in Example 28 was a light yellow solid. ¹H NMR (400 MHz, CD3OD) δ: 8.4 (s, 1H), 6.27-6.16 (m, 1H), 6.13-6.02 (m, 1H), 5.59 (dd, 1H), 4.01 (br s, 1H), 3.39 (br s, 1H), 3.17 (br s, 1H), 2.64 (br s, 1H), 2.43 (br s, 1H), 1.99-1.67 (m, 3H), 1.35 (m, 1H); LC/MS(ESI): m/z=359.1[M+H]⁺.

### Example 31

### (R)-5-(3-acrylamidopiperidin-1-yl)-2,3-dioxo-1,2,3,4-tetrahydropyrido[3,4-b]pyrazine-8-carboxamide (Compound 31)

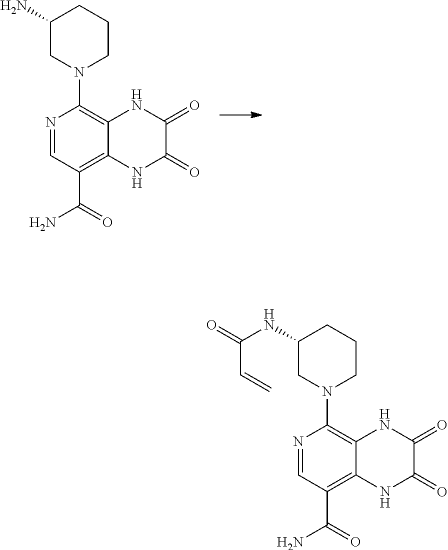 [SMILES: NC(=O)c1cnc(N2CCC[C@@H](N)C2)c2[nH]c(=O)c(=O)[nH]c12 ; C=CC(=O)N[C@@H]1CCCN(c2ncc(C(N)=O)c3[nH]c(=O)c(=O)[nH]c23)C1]

The compound 30 (250 mg, yield 65%) obtained by the intermediate (R)-5-(3-aminopiperidine)-2,3-dioxo-pyridine[3,4-b]quinoxaline-8-carboxamide and acrylic acid Example 2 was a light yellow solid. ¹H NMR (400 MHz, CD3OD) δ: 8.4 (s, 1H), 6.27-6.16 (m, 1H), 6.13-6.02(m, 1H), 5.59 (dd, 1H), 4.01 (br s, 1H), 3.39 (br s, 1H), 3.17 (br s, 1H), 2.64 (br s, 1H), 2.43 (br s, 1H), 1.99-1.67 (m, 3H), 1.35 (m, 1H); LC/MS(ESI): m/z=359.1[M+H]⁺.

### Example 32

### (S)-5-(3-(but-2-ynamido)pyrrolidin-1-yl)-2,3-dioxo-1,2,3,4-tetrahydropyrido[3,4-b]pyrazine-8-carboxamide (Compound 32)

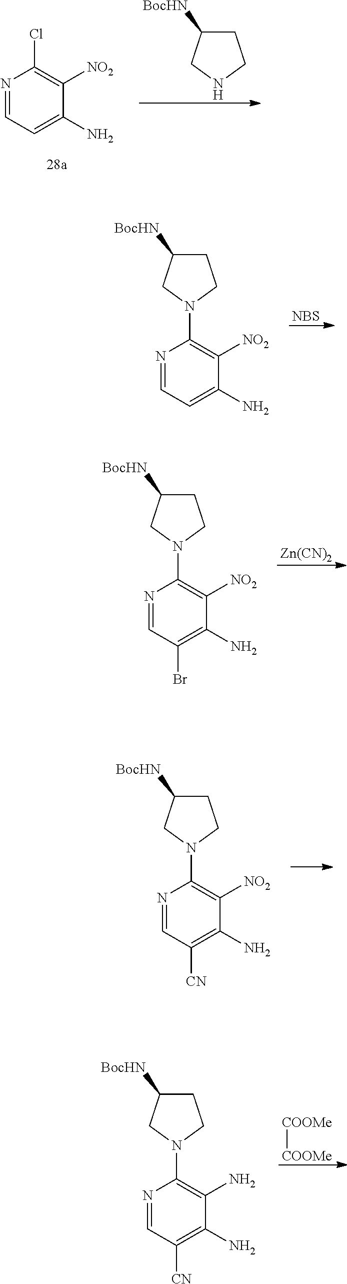

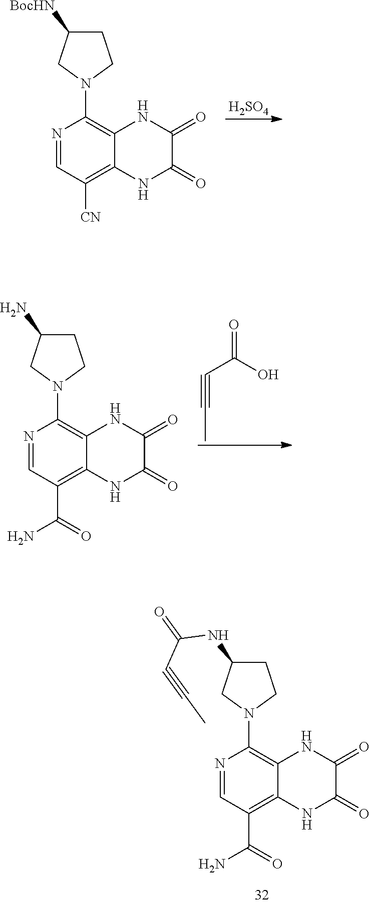

The compound 32 (224 mg, yield 58%) obtained by the method similar to example 1 (the intermediate was replaced to (S)-3-t-butyloxycarboryl aminopyrrolidine) was a light yellow solid. ¹H NMR (400 MHz, CD3OD) δ: 8.4 (s, 1H), 4.34 (m, 1H), 3.26-3.1 (m, 2H), 3.11-2.98 (m, 2H), 2.24 (m, 1H), 1.94 (s, 3H), 1.85 (m, 1H); LC/MS(ESI): m/z=357.1[M+H]⁺.

### Example 33

### (R)-5-(3-(but-2-ynamido)pyrrolidin-1-yl)-2,3-dioxo-1,2,3,4-tetrahydropyrido[3,4-b]pyrazine-8-carboxamide (Compound 32)

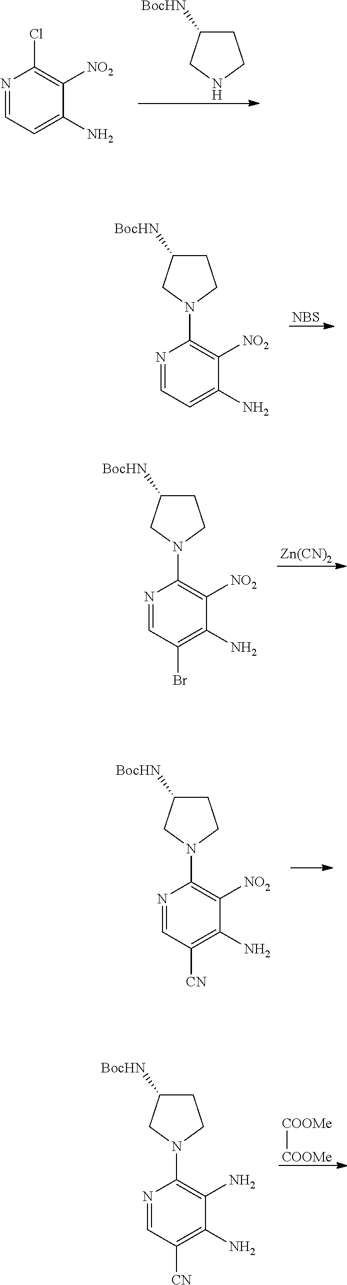

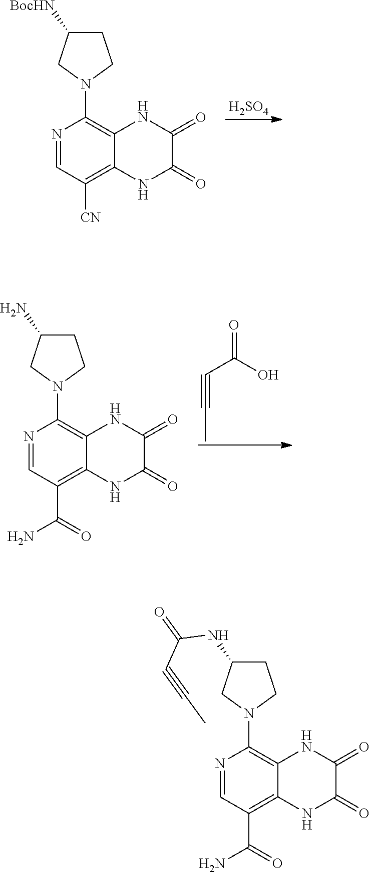

The compound 33 (259 mg, yield 67%) obtained by the method similar to example 1 (the intermediate was replaced to (R)-3-t-butyloxycarboryl aminopyrrolidine) was a light yellow solid. ¹H NMR (400 MHz, CD3OD) δ: 8.4 (s, 1H), 4.34 (m, 1H), 3.26-3.1 (m, 2H), 3.11-2.98 (m, 2H), 2.24 (m, 1H), 1.94 (s, 3H), 1.85 (m, 1H); LC/MS(ESI): m/z=357.1[M+H]⁺.

### Example 34

### (S)-5-(3-acrylamidopyrrolidin-1-yl)-2,3-dioxo-1,2,3,4-tetrahydropyrido[3,4-b]pyrazine-8-carboxamide (Compound 34)

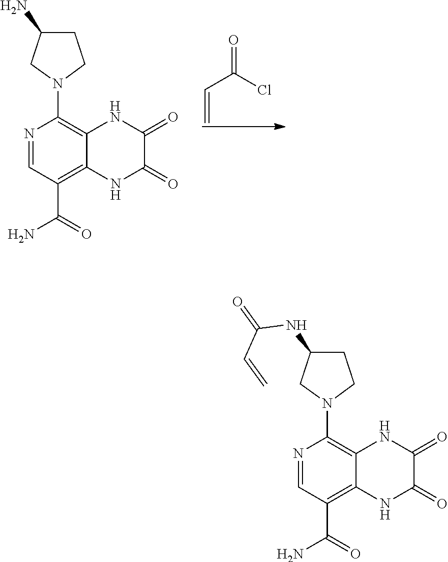

The compound 34 (221 mg, yield 59%) obtained by the intermediate (S)-5-(3-aminopiperidine)-2,3-dioxo-pyridine[3,4-b]quinoxaline-8-carboxamide and acrylic acid Example 32 was a light yellow solid. ¹H NMR (400 MHz, CD3OD) δ: 8.5 (s, 1H), 6.71-6.55 (d, 1H), 6.32-6.22 (m, 1H), 5.74 (m, 1H), 4.40-4.23 (m, 2H), 3.91 (dd, 1H), 3.86-3.58 (m, 3H), 2.24-2.02 (m, 2H); LC/MS(ESI): m/z=345.1[M+H]⁺.

### Example 35

### Preparation for (R)-5-(3-acrylamidopyrrolidin-1-yl)-2,3-dioxo-1,2,3,4-tetrahydropyrido[3,4-b]pyrazine-8-carboxamide (Compound 35)

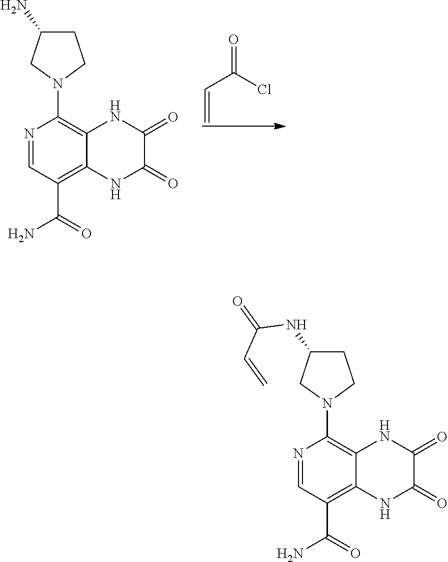

The compound 35 (221 mg, yield 59%) obtained by the intermediate (R)-5-(3-aminopiperidine)-2,3-dioxo-pyridine[3,4-b]quinoxaline-8-carboxamide and acrylic acid Example 32 was a light yellow solid. ¹H NMR (400 MHz, CD3OD) δ: 8.5 (s, 1H), 6.71-6.55 (d, 1H), 6.32-6.22 (m, 1H), 5.74 (m, 1H), 4.40-4.23 (m, 2H), 3.91 (dd, 1H), 3.86-3.58 (m, 3H), 2.24-2.02 (s, 2H); LC/MS(ESI): m/z=345.1[M+H]⁺.

### Example 36

### Preparation for (S)-4-(3-(but-2-ynamido)piperidin-1-yl)-1H-imidazo[4,5-c]pyridine-7-carboxamide (Compound 36)

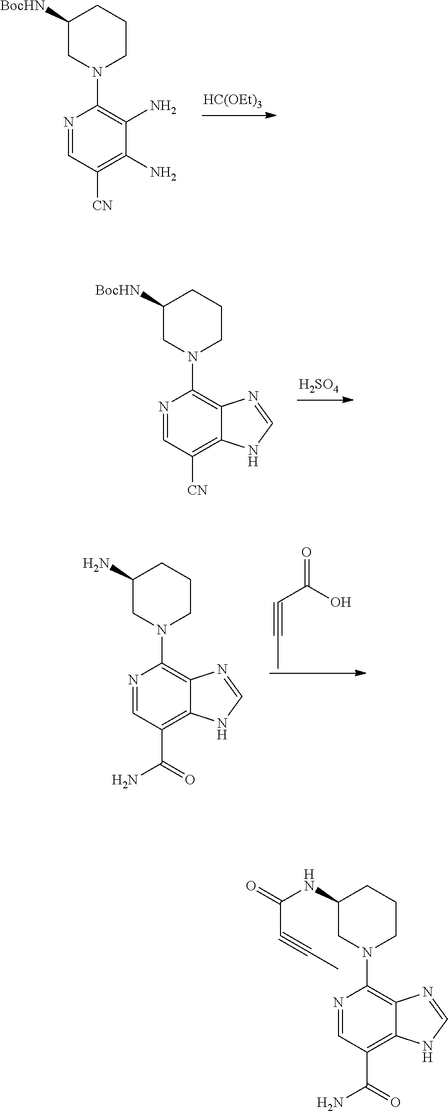

The intermediate (S)-2-(3-t-butyloxycarboryl aminopiperidine)-3,4-diamido-5 cyan-piperidine in Example 28 reacted with triethyl orthoformate, the next two steps were similar to Example 1, and the obtained compound 36 (241 mg, yield 68%) was a light yellow solid. 8.6 (s, 1H), 8.13 (s, 1H), 3.98-3.84 (m, 1H), 3.19-3.08 (m, 1H), 3.06-2.89 (m, 1H), 3.06-2.92 (m, 1H), 2.80 (br s, 1H), 1.96 (s, 3H), 1.91-1.80 (m, 1H), 1.73 (s, 2H), and 1.35 (s, 1H), LC/MS(ESI): m/z=327.1[M+H]⁺.

### Example 37

### Preparation for (S)-4-(3-(but-2-ynamido)piperidin-1-yl)-2-methyl-1H-imidazo[4,5-c]pyridine-7-carboxamide (Compound 37)

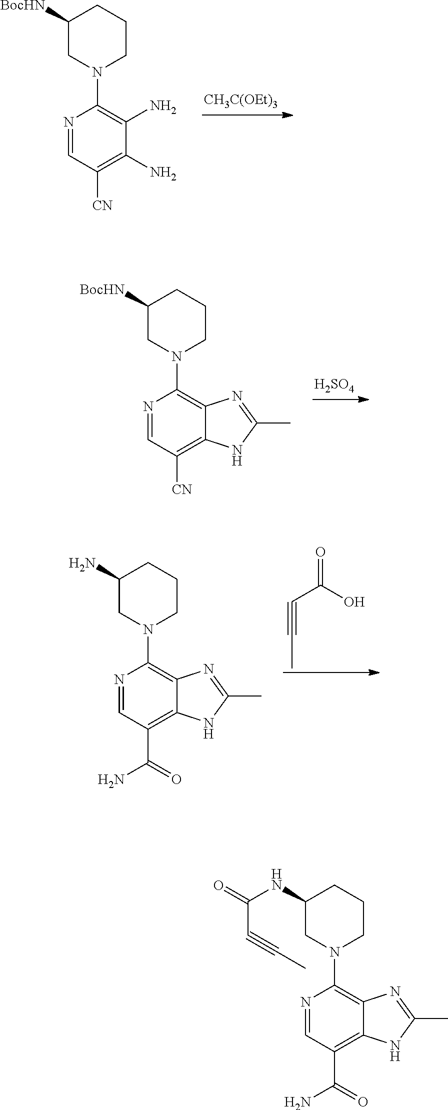

The intermediate (S)-2-(3-t-butyloxycarboryl aminopiperidine)-3,4-diamido-5 cyan-piperidine in Example 28 reacted with triethyl orthoformate, the next two steps were similar to Example 1, and the obtained compound 37 (223 mg, yield 61%) was a light yellow solid. ¹H NMR (400 MHz, CD3OD) δ: 8.7 (s, 1H), 3.98-3.84 (m, 1H), 3.20-3.07 (m, 1H), 3.07-2.89 (m, 1H), 3.05-2.91 (m, 1H), 2.80 (br s, 1H), 2.54 (s, 3H), 1.96 (s, 3H), 1.91-1.80 (m, 1H), 1.73 (s, 2H), and 1.35 (s, 1H); LC/MS(ESI): m/z=509.2[M+H]⁺.

### Example 38

### Preparation for (S)-4-(3-(but-2-ynamido)piperidin-1-yl)-2-oxo-2,3-dihydro-1H-imidazo[4,5-c]pyridine-7-carboxamide (Compound 38)

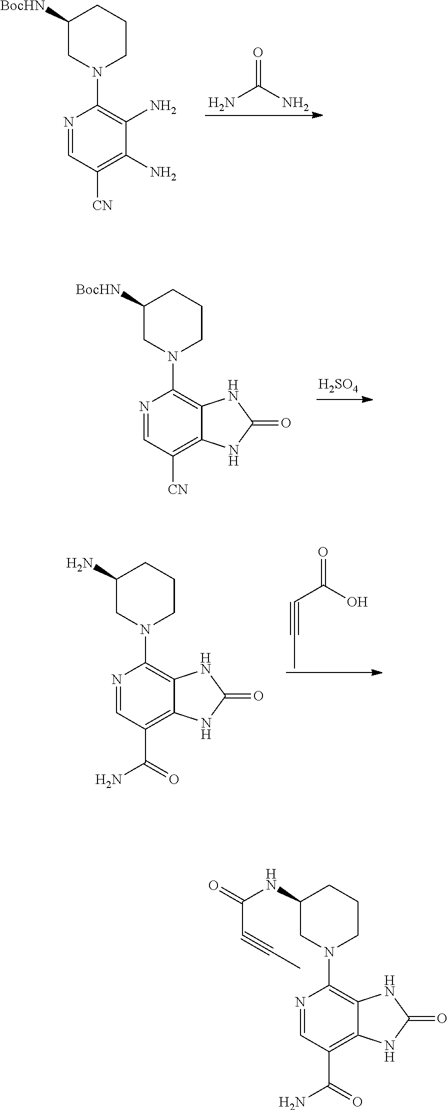

The intermediate (S)-2-(3-t-butyloxycarboryl aminopiperidine)-3,4-diamido-5 cyan-piperidine in Example 28 reacted with carbamide, the next two steps were similar to Example 1, and the obtained compound 38 (238 mg, yield 62%) was a yellow solid. ¹H NMR (400 MHz, CD3OD) δ: 8.56 (s, 1H), 3.97-3.84 (m, 1H), 3.19-3.06 (m, 1H), 3.06-2.89 (m, 1H), 3.06-2.91 (m, 1H), 2.80 (br s, 1H), 1.98 (s, 3H), 1.91-1.80 (m, 1H), 1.73 (s, 2H), and 1.36 (s, 1H); LC/MS(ESI): m/z=343.1[M+H]⁺.

### Example 39

### Preparation for (R)-4-(3-(but-2-ynamido)piperidin-1-yl)-2-oxo-2,3-dihydro-1H-imidazo[4,5-c]pyridine-7-carboxamide (Compound 39)

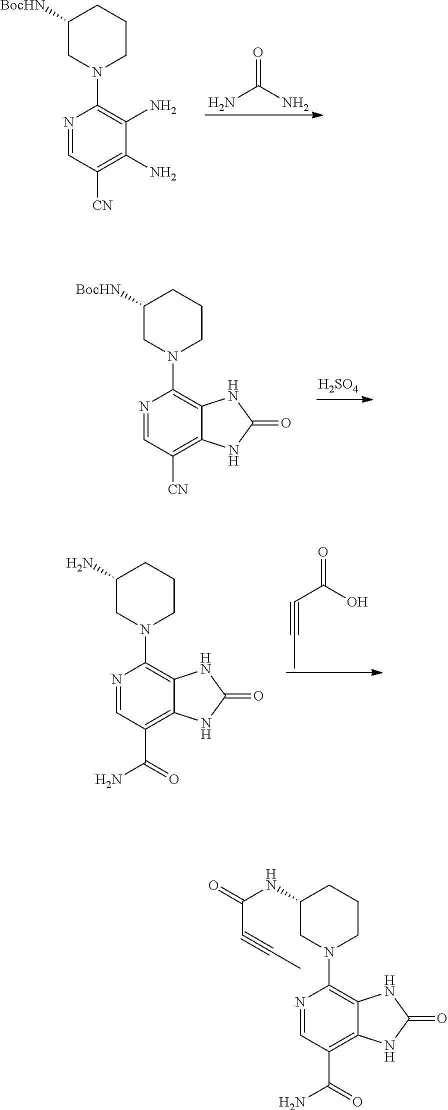

The intermediate (R)-2-(3-t-butyloxycarboryl aminopiperidine)-3,4-diamido-5 cyan-piperidine in Example 29 reacted with carbamide, the next two steps were similar to Example 1, and the obtained compound 39 (253 mg, yield 66%) was a yellow solid. ¹H NMR (400 MHz, CD3OD) δ: 8.6 (s, 1H), 3.97-3.84 (m, 1H), 3.19-3.06 (m, 1H), 3.06-2.89 (m, 1H), 3.06-2.91 (m, 1H), 2.80 (br s, 1H), 1.98 (s, 3H), 1.91-1.80 (m, 1H), 1.73 (m, 2H), 1.36 (s, 1H); LC/MS(ESI): m/z=343.1[M+H]⁺.

### Example 40

### Preparation Method 4-(1-acryloylpiperidin-4-yl)-2-oxo-2,3-dihydro-1H-imidazo[4,5-c]pyridine-7-carboxamide (Compound 40)

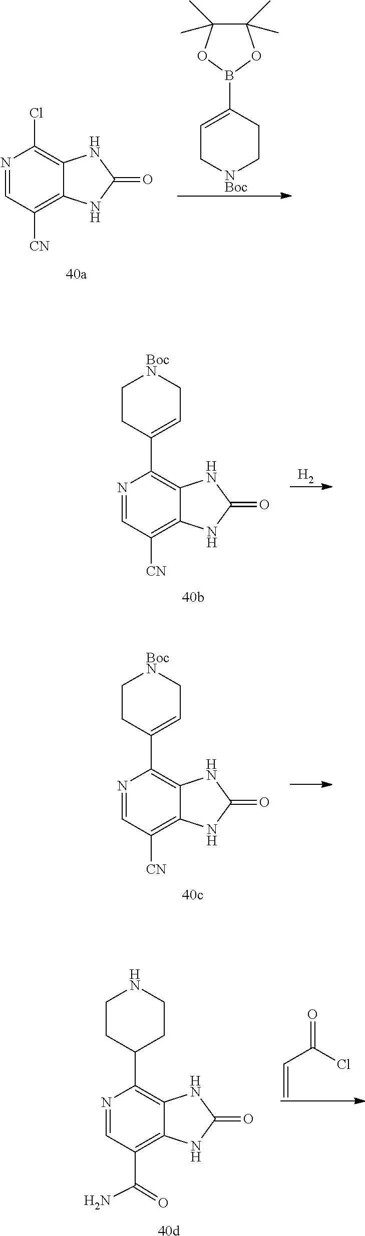

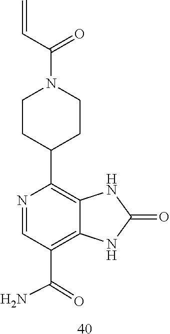

### Step 1: Synthesis of Compound 40b

The intermediate 4-chlorine-2-oxo-2,3-dihydro-1H-imidazo[4,5-c]pyridine-7-carboxamide 40b (0.975 g, 5 mmol), N-Boc-1,2,5,6-tetrahydropyridine-4-boronic acid pinacol ester (2.31 g, 7.5 mmol), potassium carbonate (2.07 g, 15 mmol), tetri (triphenylphosphine) palladium and 1,4-dioxane (100 mL) in the previous step were mixed with water (25 mL). The mixture was heated and refluxed under nitrogen protection, stirred and reacted for 16 H. The reactant was cooled to the room temperature and stirred for overnight, the reaction solution was evaporated under reduced pressure, and an off-white solid 40b (0.58 g, 35%) was obtained through column chromatography and purification. No further purification was not required for the next reaction, LC/MS(ESI): m/z=342[M+H]⁺.

### Step 2: Synthesis of Compound 40c

The solution that the compound 40b (342 mg, 1 mmol) in the previous step was stored in ethyl acetate (10 mL) and methyl alcohol (10 mL) was added with 10% Pd/C (0.1 g), and the reactant was degased for 6 times with hydrogen, and stirred for 12 h in the room temperature under the hydrogen atmosphere. The solution was filtered, the filtrate was evaporated to the brown solid crude product 40d (322 mg, 94%). No further purification was not required for the next reaction, LC/MS(ESI): m/z=344[M+H]⁺.

### Step 3: Synthesis of Compound 40d

The compound 40c (172 mg, 0.5 mmol) in the previous step was added to 80% sulfuric acid (6 mL) in batches, stirred and reacted for 2.5 H at 60° C. After cooling to the room temperature, the reaction mixture was added into ice and heated to the room stirring temperature. The pH value was regulated to 8 with KOH, it was extracted (2×15 mL) with ethyl acetate, dried with anhydrous sodium sulfate, concentrated and depressurized to obtain a brown solid intermediate 40h (1.19 g, 91%). No further purification was not required for the next reaction, LC/MS(ESI): m/z=262[M+H]⁺.

### Step 4: Synthesis of Compound 40

The compound 40d (79 mg, 0.3 mmol), triethylamine (51 mg, 0.5 mmol) and 4 ml of tetrahydrofuran were added into the reaction flask, and 0.5 ml of tetrahydrofuran solution of acryloyl chloride (45 mg, 0.5 mmol) was slowly dropped after cooling in the ice water bath. After dropping, it was continuously stirred for 4 H. The reaction solution was quenched with methyl alcohol, depressurized and dried. The residue was purified through column chromatography, and the obtained compound 40 (36 mg, yield 38%) was a yellow solid. LC/MS(ESI): m/z=316.2[M+H]⁺.

### Example 41 and Example 42

### Preparation for (S)-4-(1-acryloylamino piperidine-3-yl)-2-methyl-1H-imidazo[4,5-c]pyridine-7-carboxamide (Compound 41) and (R)-4-(1-acryloylamino piperidine-3-yl)-2-methyl-1H-imidazo[4,5-c]pyridine-7-carboxamide (Compound 42)

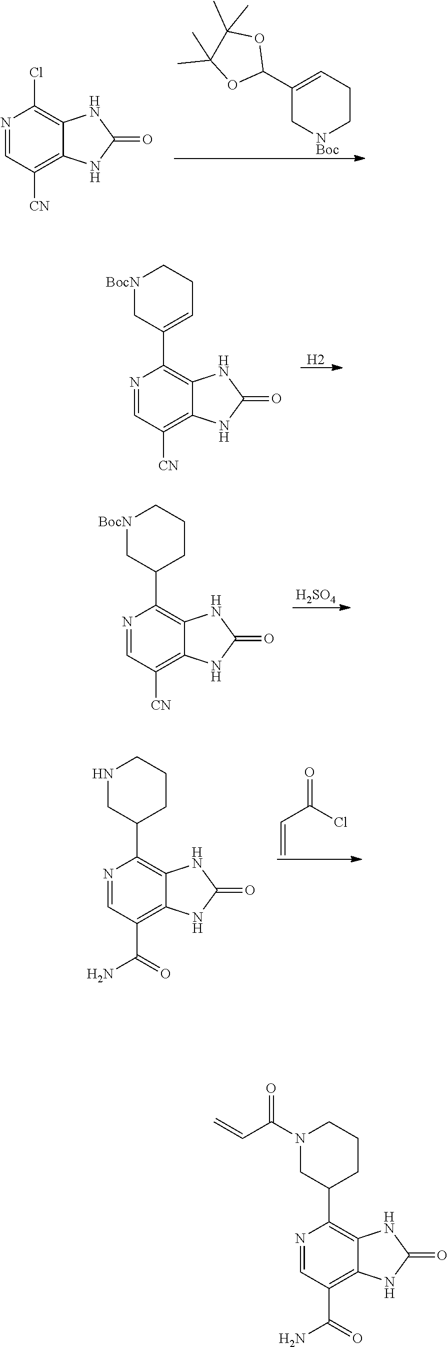

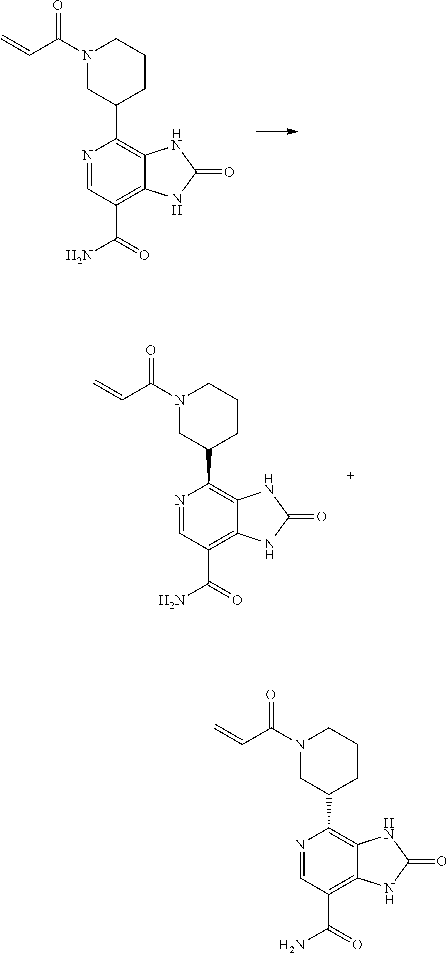

The racemate (R,S)-4-(1-acryloylamino piperidine-3-yl)-2-methyl-1H-imidazo[4,5-c]pyridine-7-carboxamide (46 mg, yield 48%, this is the last step, the same below) obtained through the method similar to Example 40 (the intermediate was replaced to 1-t-butyloxycarboryl-3,6-dihydro-2H-pyridine-5-boronic acid pinacol ester was a light yellow solid, LC/MS(ESI): m/z=316.1[M+H]⁺.

The racemate (R,S)-4-(1-acryloylamino piperidine-3-yl)-2-methyl-1H-imidazo[4,5-c]pyridine-7-carboxamide can obtain the compounds 41 and 42 through chiral column separation. The MS (ESI) of the two was consistent with the racemate.

### Example 43 Biological Activity Test

The following further describes and explains the present disclosure in combination with test examples, but these implementations do not mean to limit the scope of the present disclosure.

### Experimental Example 1: BTK Kinase Test

### 1. Assay Methods

Dilute 350 ng/uL BTK mother solution with kinase buffer solution (50 mM HEPES, 10 mM MgCl₂, 2 mM DTT, 1 mM EGTA, 0.01% Tween 20), and add 6 μL 1.67×0.134 ng/μL working solution (final concentration 0.08 ng/μL), using the nanoliter sampler, add different compounds dissolved by DMSO into the pores, so that the final concentration of the compound is 1000 nM-0.244 nM, and the final concentration of the positive drug is 50 nM-0.0122 nM. Four times the gradient, a total of seven concentrations are available. At the same time, set up blank control pores (without enzyme) and negative control pores (with enzyme, with solvent DMSO), and set up two double pores. After the enzyme reacts with compound or solvent for 30 min, 5×250 μM ATP (final concentration is 50 μM) and 5×0.5 μM Substrate (final concentration 0.1 μμM. ULight poly GT), mix at 1:1, 4 per hole μL is added into the hole; after sealing, add 5% to each hole after reacting at room temperature for 2 hours μL 4×8 nM detection reagent (final concentration is 2 nM, Ab), incubate at room temperature for 1 hour; PE instrument reading board (excitation 620 nm, emission 665 nm). Calculate the inhibition rate and calculate the IC₅₀ value.

### 2. Assay Results

The compound of the application can strongly inhibit BTK activity. Table 1 lists the activity of representative compounds of the invention in BTK detection. In these tests, the following levels are used: for IC50, “A” means IC50≤10 nM; “B” means 10<IC50≤100 nM; “C” means 100<IC50≤500 nM; “D” means 500<IC50≤2000 nM.

TABLE 1
BTK kinase inhibitory activity

| NO | BTK IC₅₀ (nM) |
|---|---|
| 1 | A |
| 2 | A |
| 3 | A |
| 4 | A |
| 5 | A |
| 6 | A |
| 7 | A |
| 8 | A |
| 9 | A |
| 18 | A |
| 19 | A |
| 20 | A |
| 21 | A |
| 22 | A |
| 23 | A |
| 24 | A |
| 25 | A |
| 26 | A |
| 27 | A |
| 28 | A |
| 36 | A |
| 38 | A |
| 40 | A |
| 41 | A |
| 42 | A |

### Experimental Example 2: Tumor Cell Proliferation Inhibition Assay

### 1. Assay Methods

Cell viability was evaluated by measuring a content of triphosadenine (ATP) by using CellTiter-Glo luminescent cell viability assay kit method (Promega, #G7572, Madison, WI). Diffuse large B cell lymphoma cell strain TMD-8 cell strain is purchased from American Type Culture Collection (ATCC). After the cells were digested from a cell culture dish by pancreatin and re-suspended by DPBS culture medium, a Scepter automatic cell counter (Millipore, #PHCC00000) was used for counting to measure a cell density. The cells were diluted to be a solution containing 44,000 cells per milliliter. The cell solution with the adjusted density was added into a cell assay plate with 90 μl per well. The well plate was placed to an incubator with 5% CO2 at 37° C. to be incubated for 24 hours and then added with the compound to be tested with different concentrations. The cells and the compound were together incubated for 72 hours in the presence of 10% fetal calf serum. The content of ATP was measured by using CellTiter-Glo® Luminescent Cell Viability Assay kit (referring to manufacturer's instruction) to evaluate the inhibition to the cell growth. Briefly speaking, each well was added with 30 μl of CellTiter-Glo® reagent, the plate was shaken for 10 minutes to induce the cells to be lysed, and fluorescence signal was detected and recorded by fluorescence/chemiluminescence analyzer Fluoroskan Ascent FL (Thermo Scientific Fluoroskan Ascent FL). The maximal signal value was obtained from cells processed by dimethyl sulfoxide (DMSO) for 72 or 120 hours. The minimal signal value obtained from separate culture medium (wherein the number of cells was zero) was defined as 0. Inhibition ratio %=(maximal signal value−compound signal value)/(maximal signal value−minimal signal value)×100%. The data was processed by using GraphPad Prism V5.0 (GraphPad Software, San Diego, CA) software. IC₅₀ value was fitted and calculated via S-shaped dose-response curve.

### 2. Assay Results

The compound of the application can strongly inhibit BTK activity. Table 1 lists the activity of representative compounds of the invention in BTK detection. In these tests, the following levels are used: for IC50, “A” means IC50≤10 nM; “B” means 10<IC50≤100 nM; “C” means 100<IC50≤500 nM; “D” means 500<IC50≤2000 nM.

TABLE 2
tumor cell proliferation inhibition results

| NO | TMD-8 IC₅₀ (nM) |
|---|---|
| 23 | A |
| 25 | A |
| 26 | A |
| 27 | A |
| 38 | A |
| 40 | A |
| 41 | A |
| 42 | A |

Although the present disclosure is described in detail above, those skilled in the art may understand that various modifications and changes may be made to the present disclosure under a premise of without deviating from the spirt and scope of the present disclosure. The claim scope of the present disclosure is not limited to the above detail description, but belongs to the claims.

1. A compound represented by a general formula (I), a stereoisomer, a pharmaceutically acceptable salt, a polymorph or an isomer thereof, wherein the compound represented by the general formula (I) is as follows: 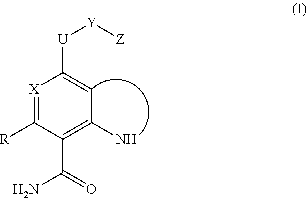
in the formula:
R is independently selected from 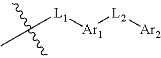
H or D;
L₁ is independently selected from —C₀₋₄alkyl-, —CR¹R²—, —C₁₋₂alkyl(R¹)(OH)—, —C(O)—, —CR¹R²O—, —OCR¹R²—, —SCR¹R²—, —CR¹R²S—, —NR¹—, —NR¹C(O)—, —C(O)NR¹—, —NR¹C(O)NR²—, —CF₂—, —O—, —S—, —S(O)ₘ—, —NR¹S(O)₂—, —S(O)₂NR¹—;
L₂ is independently selected from —C₀₋₄alkyl-, —CR¹R²—, —C₁₋₂alkyl(R¹)(OH)—, —C(O)—, —CR¹R²O—, —OCR¹R²—, —SCR¹R²—, —CR¹R²S—, —NR¹—, —NR¹C(O)—, —C(O)NR¹—, —NR¹C(O)NR²—, —CF₂—, —O—, —S—, —S(O)ₘ—, —NR¹S(O)₂—, —S(O)₂NR¹—;
Ar₁ and Ar₂ are independently selected from phenyl, 5-6 membered heteroaryl, and the phenyl and the 5-6 membered heteroaryl are optionally substituted with one or more G¹;
X is independently selected from N, CR³; 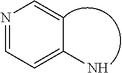
is independently selected from the structure below: 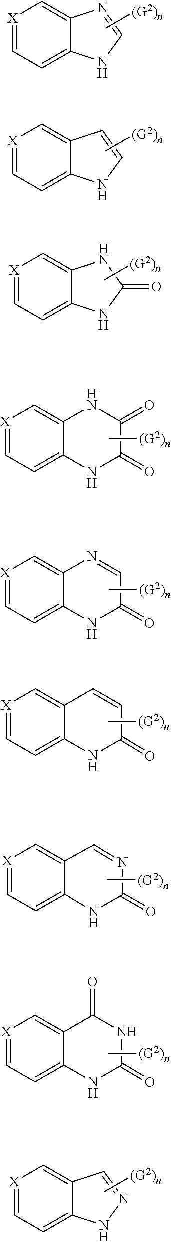
n is 1 or 2;
U is independently selected from bond, —C₁₋₄alkyl-, —CR⁴R⁵—, —C₁₋₂alkyl(R⁴)(OH)—, —C(O)—, —CR⁴R⁵O—, —OCR⁴R⁵—, —SCR⁴R⁵—, —CR⁴R⁵S—, —NR⁴—, —NR⁴C(O)—, —C(O)NR⁴—, —NR⁴C(O)NR⁵—, —CF₂—, —O—, —S—, —S(O)ₘ—, —NR⁴S(O)₂—, —S(O)₂NR⁴—;
Y is absent or selected from C₃₋₈cycloalkyl, 3-8 membered heterocycloalkyl, 5-12 membered bicycloalkyl, 5-12 membered heterobicycloalkyl, 5-12 membered spirocycloalkyl, 5-12 membered heterospirocycloalkyl, aryl or heteroaryl, and the C₃₋₈cycloalkyl, 3-8 membered heterocycloalkyl, 5-12 membered bicycloalkyl, 5-12 membered heterobicycloalkyl, 5-12 membered spirocycloalkyl, 5-12 membered heterospirocycloalkyl, aryl and heteroaryl are optionally substituted with one or more G³;
Z is independently selected from CN, —NR¹²CN, 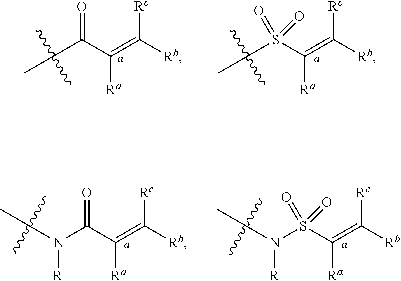
a is a double bond or a triple bond;
when a is a double bond, R^{a}, R^{b}, and R^{c} are independently selected from H, D, —CN, halogen, C₁₋₆alkyl, C₃₋₆cycloalkyl or C₃₋₆heterocycloalkyl, and the C₁₋₆alkyl, C₃₋₆cycloalkyl and C₃₋₆heterocycloalkyl are optionally substituted with one or more G⁴;
when a is a triple bond, R^{a} and R^{c} are absent, R^{b} is independently selected from H, D, —CN, halogen, C₁₋₆alkyl, C₃₋₆cycloalkyl or 3-6 heterocycloalkyl, and the C₁₋₆alkyl, C₃₋₆cycloalkyl and 3-6 heterocycloalkyl are optionally substituted with one or more G⁵;
R¹² is independently selected from H, D, C₁₋₆alkyl, C₃₋₆cycloalkyl or C₃₋₆heterocycloalkyl, and C₁₋₆alkyl, C₃₋₆cycloalkyl and C₃₋₆heterocycloalkyl are optionally substituted with one or more G⁶;
G¹, G², G³, G⁴, G⁵, and G⁶ are each independently selected from H, D, —CN, halogen, C₁₋₆alkyl, C₂₋₆alkenyl, C₂₋₆alkynyl, C₃₋₈cycloalkyl or 3-8 heterocycloalkyl, C₆₋₁₀ membered aryl, 5-10 membered heteroaryl, —OR⁶, —OC(O)NR⁶R⁷, —C(O)OR⁶, —C(O)NR⁶R⁷, —C(O)R⁶, —NR⁶R⁷, —NR⁶C(O)R⁷, —NR⁶C(O)NR⁷R⁸, —S(O)ₘR⁶ or —NR⁶S(O)ₘR⁷, C₁₋₆alkyl, C₂₋₆alkenyl, C₂₋₆alkynyl, C₃₋₈cycloalkyl, 3-8 heterocycloalkyl, C₆₋₁₀ membered aryl and 5-10 membered heteroaryl are optionally substituted with the substituent of one or more CNs, halogen, C₁₋₆alkyl, C₂₋₆alkenyl, C₂₋₆alkynyl, C₃₋₈cycloalkyl or 3-8 heterocycloalkyl, C₆₋₁₀ membered aryl, 5-10 membered heteroaryl, —OR⁹, —OC(O)NR⁹R¹⁰, —C(O)OR⁹, —C(O)NR⁹R¹⁰, —C(O)R⁹, —NR⁹R¹⁰, —NR⁹C(O)R¹⁰, —NR⁹C(O)NR¹⁰R¹¹, —S(O)ₘR⁹ or —NR⁹S(O)ᵢR¹⁰;
R³, R⁴, R⁵, R⁶, R⁷, R⁸, R⁹, R¹⁰ and R¹¹ are each independently selected from H, D, —CN, halogen, C₁₋₆alkyl-, C₃₋₈cycloalkyl or 3-8 heterocycloalkyl, aryl or heteroaryl; and
m is 1 or 2.

2. The compound represented by the general formula (I), the pharmaceutically acceptable salt or the stereoisomer thereof according to claim 1, wherein the general formula (I) is further represented by the general formula Ia: 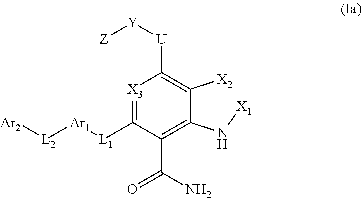
in the formula:
X₁, X₂, X₃ are independently selected from N, CR¹;
Ar₁ and Ar₂ are independently selected from phenyl, 5-6 membered heteroaryl, and the phenyl and 5-6 membered heteroaryl are optionally substituted with one or more G¹;
R¹ is independently selected from H, D, —CN, halogen, C₁₋₆alkyl, COOH, CONH₂, NHCOH, CONHR², OR² or —NHR²;
R² is independently selected from H, D, —CN, halogen, C₁₋₆alkyl, C₃₋₆cycloalkyl, 3-6 heterocycloalkyl, —OR³, —NR³R⁴ and —C(O)NR³R⁴, and the C₁₋₆alkyl, C₃₋₆cycloalkyl or 3-6 heterocycloalkyl is optionally substituted with —CN, halogen, —OR⁵, —NR⁵R⁶, C₁₋₆alkyl, C₃₋₆cycloalkyl, or 3-6 heterocycloalkyl;
L₁, L₂ and U are independently selected from bond, —C₁₋₄alkyl-, —CR⁷R⁸—, —C₁₋₂alkyl(R⁷)(OH)—, —C(O)—, —CR⁷R⁸O—, —OCR⁷R⁸—, —SCR⁷R⁸—, —CR⁷R⁸S—, —NR⁷—, —NR⁷C(O)—, —C(O)NR⁷—, —NR⁷C(O)NR⁸—, —CF₂—, —O—, —S—, —S(O)ₘ—, —NR⁷S(O)₂—, —S(O)₂NR⁸—;
Y is absent or selected from C₃₋₈cycloalkyl, 3-8 membered heterocycloalkyl, 5-12 membered bicycloalkyl, 5-12 membered heterobicycloalkyl, 5-12 membered spirocycloalkyl, 5-12 membered heterospirocycloalkyl, aryl or heteroaryl, and the C₃₋₈cycloalkyl, 3-8 membered heterocycloalkyl, 5-12 membered bicycloalkyl, 5-12 membered heterobicycloalkyl, 5-12 membered spirocycloalkyl, 5-12 membered heterospirocycloalkyl, aryl or heteroaryl is optionally substituted with one or more G²;
Z is independently selected from CN, —NR¹²CN, 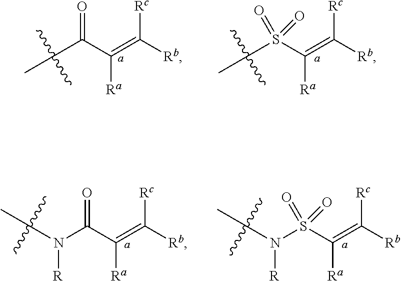
bond c is a double bond or a triple bond;
when c is a double bond, R^{a}, R^{b}, and R^{c} are each independently selected from H, —CN, halogen, C₁₋₆alkyl, C₃₋₆cycloalkyl or 3-6 heterocycloalkyl, and the C₁₋₆alkyl, C₃₋₆cycloalkyl and 3-6 heterocycloalkyl are optionally substituted with one or more G³;
R^{a} and R^{b} or R^{b} and R^{c} optionally form a 3-6 membered ring containing heteroatoms with their connected carbon atoms;
when the bond c is a triple bond, R^{a} and R^{c} are absent, R^{b} is independently selected from H and —CN, and the halogen, C₁₋₆alkyl, C₃₋₆cycloalkyl or 3-6 heterocycloalkyl is optionally substituted with one or more G⁴;
R⁹ is independently selected from H, C₁₋₆alkyl, C₃₋₆cycloalkyl or 3-6 heterocycloalkyl, and the C₁₋₆alkyl, C₃₋₆cycloalkyl and 3-6 heterocycloalkyl are optionally substituted with one or more G⁵;
G¹, G², G³, G⁴ and G⁵ are each independently selected from —CN, halogen, C₁₋₆alkyl, C₂₋₆alkenyl, C₂₋₆alkynyl, C₃₋₈cycloalkyl or 3-8 heterocycloalkyl, C₆₋₁₀ membered aryl, 5-10 membered heteroaryl, —OR¹⁰, —OC(O)NR¹⁰R¹¹, —C(O)OR¹⁰, —C(O)NR¹⁰R¹¹, —C(O)R¹⁰, —NR¹⁰R¹¹, —NR¹⁰C(O)R¹¹, —NR¹⁰C(O)NR¹¹R¹², —S(O)ₘR¹⁰ or —NR¹⁰S(O)ₘR¹¹, and the C₁₋₆alkyl, C₂₋₆alkenyl, C₂₋₆alkynyl, C₃₋₈cycloalkyl or 3-8 heterocycloalkyl, C₆₋₁₀ membered aryl, 5-10 membered heteroaryl are optionally substituted with the substituents of one or more CNs, halogen, C₁₋₆alkyl, C₂₋₆alkenyl, C₂₋₆alkynyl, C₃₋₈cycloalkyl or 3-8 heterocycloalkyl, C₆₋₁₀ membered aryl, 5-10 membered heteroaryl, —OR¹³, —OC(O)NR¹³R¹⁴, —C(O)OR¹³, —C(O)NR¹³R¹⁴, —C(O)R¹³, —NR¹³R¹⁴, —NR¹³C(O)R¹⁴, —NR¹³C(O)NR¹⁴R¹⁵, —S(O)ₘR¹³ or —NR¹³S(O)ᵢR¹⁴;
R³, R⁴, R⁵, R⁶, R⁷, R⁸, R⁹, R¹⁰, R¹¹, R¹³, R¹⁴ and R¹⁵ are each independently selected from H, —CN, halogen, C₁₋₆alkyl-, C₃₋₈cycloalkyl or 3-8 heterocycloalkyl, aryl or heteroaryl; and
m is 1 or 2;

3. The compound represented by the general formula (I), the pharmaceutically acceptable salt or the stereoisomer thereof according to claim 1, wherein the general formula (I) is further represented by the general formula Ib: 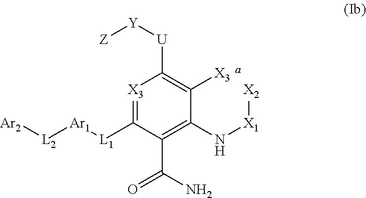
in the formula:
X₁, X₂, X₃, X₄ are independently selected from N, CR¹;
Bonds a and b are single or double bonds;
Ar₁ and Ar₂ are independently selected from phenyl, 5-6 membered heteroaryl, and the phenyl and 5-6 membered heteroaryl are optionally substituted with one or more G¹;
R¹ is independently selected from H, D, —CN, halogen, C₁₋₆alkyl, COOH, CONH₂, NHCOH, CONHR², OR² or —NHR²;
R² is independently selected from H, D, —CN, halogen, C₁₋₆alkyl, C₃₋₆cycloalkyl, 3-6 heterocycloalkyl, —OR³, —NR³R⁴ and —C(O)NR³R⁴, and the C₁₋₆alkyl, C₃₋₆cycloalkyl or 3-6 heterocycloalkyl is optionally substituted with —CN, halogen, —OR⁵, —NR⁵R⁶, C₁₋₆alkyl, C₃₋₆cycloalkyl, or 3-6 heterocycloalkyl;
L₁, L₂ and U are independently selected from bond, —C₁₋₄alkyl-, —CR⁷R⁸—, —C₁₋₂alkyl(R⁷)(OH)—, —C(O)—, —CR⁷R⁸O—, —OCR⁷R⁸—, —SCR⁷R⁸—, —CR⁷R⁸S—, —NR⁷—, —NR⁷C(O)—, —C(O)NR⁷—, —NR⁷C(O)NR⁸—, —CF₂—, —O—, —S—, —S(O)ₘ—, —NR⁷S(O)₂—, —S(O)₂NR⁸—;
Y is absent or selected from C₃₋₈cycloalkyl, 3-8 membered heterocycloalkyl, 5-12 membered bicycloalkyl, 5-12 membered heterobicycloalkyl, 5-12 membered spirocycloalkyl, 5-12 membered heterospirocycloalkyl, aryl or heteroaryl, and the C₃₋₈cycloalkyl, 3-8 membered heterocycloalkyl, 5-12 membered bicycloalkyl, 5-12 membered heterobicycloalkyl, 5-12 membered spirocycloalkyl, 5-12 membered heterospirocycloalkyl, aryl or heteroaryl is optionally substituted with one or more G²;
Z is independently selected from CN, —NR¹²CN, 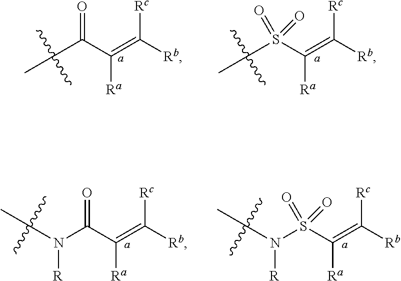
Bond c is a double bond or a triple bond;
when c is double bond, R^{a}, R^{b}, and R^{c} are each independently selected from H, —CN, halogen, C₁₋₆alkyl, C₃₋₆cycloalkyl or 3-6 heterocycloalkyl, and the C₁₋₆alkyl, C₃₋₆cycloalkyl and 3-6 heterocycloalkyl are optionally substituted with one or more G³;
R^{a} and R^{b} or R^{b} and R^{c} optionally form a 3-6 membered ring containing heteroatoms with their connected carbon atoms;
when c is a triple bond, R^{a} and R^{c} are absent, R^{b} is independently selected from H and —CN, and the halogen, C₁₋₆alkyl, C₃₋₆cycloalkyl or 3-6 heterocycloalkyl is optionally substituted with one or more G⁴;
R⁹ is independently selected from H, C₁₋₆alkyl, C₃₋₆cycloalkyl or 3-6₆heterocycloalkyl, and the C₁₋₆alkyl, C₃₋₆cycloalkyl and 3-6 heterocycloalkyl are optionally substituted with one or more G⁵;
G¹, G², G³, G⁴ and G⁵ are each independently selected from —CN, halogen, C₁₋₆alkyl, C₂₋₆alkenyl, C₂₋₆alkynyl, C₃₋₈cycloalkyl or 3-8 heterocycloalkyl, C₆₋₁₀ membered aryl, 5-10 membered heteroaryl, —OR¹⁰, —OC(O)NR¹⁰R¹¹, —C(O)OR¹⁰, —C(O)NR¹⁰R¹¹, —C(O)R¹⁰, —NR¹⁰R¹¹, —NR¹⁰C(O)R¹¹, —NR¹⁰C(O)NR¹¹R¹², —S(O)ₘR¹⁰ or —NR¹⁰S(O)ₘR¹¹, and the C₁₋₆alkyl, C₂₋₆alkenyl, C₂₋₆alkynyl, C₃₋₈cycloalkyl, 3-8 heterocycloalkyl, C₆₋₁₀ membered aryl and 5-10 membered heteroaryl are optionally substituted with substituents of one or more —CN, halogen, C₁₋₁₀alkyl, C₂₋₁₀alkenyl, C₂₋₁₀alkynyl, C₃₋₁₂cycloalkyl or 3-8 heterocycloalkyl, C₆₋₁₀ membered aryl, 5-10 membered heteroaryl, —OR¹³, —OC(O)NR¹³R¹⁴, —C(O)OR¹³, —C(O)NR¹³R¹⁴, —C(O)R¹³, —NR¹³R¹⁴, —NR¹³C(O)R¹⁴, —NR¹³C(O)NR¹⁴R¹⁵, —S(O)ₘR¹³ or —NR¹³S(O)ᵢR¹⁴;
R³, R⁴, R⁵, R⁶, R⁷, R⁸, R⁹, R¹⁰, R¹¹, R¹³, R¹⁴ and R¹⁵ are each independently selected from H, —CN, halogen, C₁₋₆alkyl-, C₃₋₈cycloalkyl or 3-8 heterocycloalkyl, aryl or heteroaryl; and
m is 1 or 2.

4. The compound represented by the general formula (I), the pharmaceutically acceptable salt or the stereoisomer thereof according to claim 1, wherein the general formula (I) is further represented by the general formula Ic: 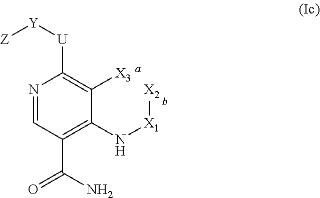
in the formula:
X₁, X₂ are independently selected from N, CR¹;
X₃ is absent or independently selected from N, CR¹;
bonds a and b are single or double bonds;
R¹ is independently selected from H, D, —CN, halogen, C₁₋₆alkyl, COOH, CONH₂, NHCOH, CONHR², OR² or —NHR²;
R² is independently selected from H, D, —CN, halogen, C₁₋₆alkyl, C₃₋₆cycloalkyl, 3-6 heterocycloalkyl, —OR³, —NR³R⁴ and —C(O)NR³R⁴, and the C₁₋₆alkyl, C₃₋₆cycloalkyl or 3-6 heterocycloalkyl is optionally substituted with —CN, halogen, —OR⁵, —NR⁵R⁶, C₁₋₆alkyl, C₃₋₆cycloalkyl or 3-6 heterocycloalkyl;
U is independently selected from bond, —C₁₋₄alkyl-, —CR⁷R⁸—, —C₁₋₂alkyl(R⁷)(OH)—, —C(O)—, —CR⁷R⁸O—, —OCR⁷R⁸—, —SCR⁷R⁸—, —CR⁷R⁸S—, —NR⁷—, —NR⁷C(O)—, —C(O)NR⁷—, —NR⁷C(O)NR⁸—, —CF₂—, —O—, —S—, —S(O)ₘ—, —NR⁷S(O)₂—, —S(O)₂NR⁸—;
Y is absent or selected from C₃₋₈cycloalkyl, 3-8 membered heterocycloalkyl, 5-12 membered bicycloalkyl, 5-12 membered heterobicycloalkyl, 5-12 membered spirocycloalkyl, 5-12 membered heterospirocycloalkyl, aryl or heteroaryl, and the C₃₋₈cycloalkyl, 3-8 membered heterocycloalkyl, 5-12 membered bicycloalkyl, 5-12 membered heterobicycloalkyl, 5-12 membered spirocycloalkyl, 5-12 membered heterospirocycloalkyl, aryl or heteroaryl is optionally substituted with one or more G¹;
Z is independently selected from CN, —NR¹²CN, 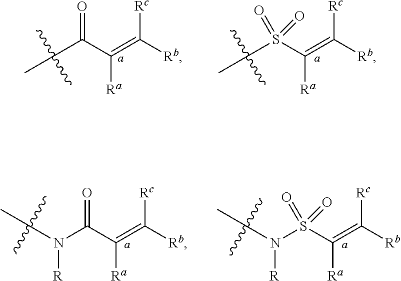
bond c is a double bond or a triple bond;
when c is a double bond, R^{a}, R^{b}, and R^{c} are each independently selected from H, —CN, halogen, C₁₋₆alkyl, C₃₋₆cycloalkyl or 3-6 heterocycloalkyl, and the C₁₋₆alkyl, C₃₋₆cycloalkyl and 3-6 heterocycloalkyl are optionally substituted with one or more G²;
R^{a} and R^{b} or R^{b} and R^{c} optionally form a 3-6 membered ring containing heteroatoms with their connected carbon atoms;
when bond c is a triple bond, R^{a} and R^{c} are absent, R^{b} is independently selected from H and —CN, and the halogen, C₁₋₆alkyl, C₃₋₆cycloalkyl or 3-6 heterocycloalkyl is optionally substituted with one or more G³;
R⁹ is independently selected from H, C₁₋₆alkyl, C₃₋₆cycloalkyl or 3-6 heterocycloalkyl, and the C₁₋₆alkyl, C₃₋₆cycloalkyl and 3-6 heterocycloalkyl are optionally substituted with one or more G⁴;
G¹, G², G³ and G⁴ are each independently selected from —CN, halogen, C₁₋₆alkyl, C₂₋₆alkenyl, C₂₋₆alkynyl, C₃₋₈cycloalkyl or 3-8 heterocycloalkyl, C₆₋₁₀ membered aryl, 5-10 membered heteroaryl, —OR¹⁰, —OC(O)NR¹⁰R¹¹, —C(O)OR¹⁰, —C(O)NR¹⁰R¹¹, —C(O)R¹⁰, —NR¹⁰R¹¹, —NR¹⁰C(O)R¹¹, —NR¹⁰C(O)NR¹¹R¹², —S(O)ₘR¹⁰ or —NR¹⁰S(O)ₘR¹¹, and the C₁₋₆alkyl, C₂₋₆alkenyl, C₂₋₆alkynyl, C₃₋₈cycloalkyl, 3-8 heterocycloalkyl, C₆₋₁₀ membered aryl and 5-10 membered heteroaryl are optionally substituted with the substituents of one or more —CN, halogen, C₁₋₆alkyl, C₂₋₆alkenyl, C₂₋₆alkynyl, C₃₋₈cycloalkyl or 3-8 heterocycloalkyl, C₆₋₁₀ membered aryl, 5-10 membered heteroary, —OR¹³, —OC(O)NR¹³R¹⁴, —C(O)OR¹³, —C(O)NR¹³R¹⁴, —C(O)R¹³, —NR¹³R¹⁴, —NR¹³C(O)R¹⁴, —NR¹³C(O)NR¹⁴R¹⁵, —S(O)ₘR¹³ or —NR¹³S(O)ₘR¹⁴;
R³, R⁴, R⁵, R⁶, R⁷, R⁸, R⁹, R¹⁰, R¹¹, R¹³, R¹⁴ and R¹⁵ are each independently selected from H, —CN, halogen, C₁₋₆alkyl-, C₃₋₈cycloalkyl or 3-8 heterocycloalkyl, aryl or heteroaryl; and
m is 1 or 2;

5. The compound, the pharmaceutically acceptable salt or the stereoisomer thereof according to claim 1, wherein the compound is selected from:

| | |
|---|---|
| 1 | (S)-4-(3-(but-2-ynamido)piperidin-1-yl)-2-oxo-6-(4-phenoxyphenyl)-2,3-dihydro- |
| | 1H-imidazo[4,5-c]pyridine-7-carboxamide |
| 2 | (S)-4-(3-(but-2-ynamido)piperidin-1-yl)-6-(4-phenoxyphenyl)-1H-pyrazolo[4,3- |
| | c]pyridine-7-carboxamide |
| 3 | (S)-4-(3-acrylamidopiperidin-1-yl)-2-oxo-6-(4-phenoxyphenyl)-2,3-dihydro-1H- |
| | imidazo[4,5-c]pyridine-7-carboxamide |
| 4 | (S)-4-(3-acrylamidopyrrolidin-1-yl)-2-oxo-6-(4-phenoxyphenyl)-2,3-dihydro-1H- |
| | imidazo[4,5-c]pyridine-7-carboxamide |
| 5 | (S)-4-(3-(but-2-ynamido)pyrrolidin-1-yl)-2-oxo-6-(4-phenoxyphenyl)-2,3- |
| | dihydro-1H-imidazo[4,5-c]pyridine-7-carboxamide |
| 6 | (R)-4-(3-(but-2-ynamido)piperidin-1-yl)-2-oxo-6-(4-phenoxyphenyl)-2,3-dihydro- |
| | 1H-imidazo[4,5-c]pyridine-7-carboxamide |
| 7 | (R)-4-(3-(but-2-ynamido)piperidin-1-yl)-6-(4-phenoxyphenyl)-1H-pyrazolo[4,3- |
| | c]pyridine-7-carboxamide |
| 8 | (R)-4-(3-acrylamidopiperidin-1-yl)-2-oxo-6-(4-phenoxyphenyl)-2,3-dihydro-1H- |
| | imidazo[4,5-c]pyridine-7-carboxamide |
| 9 | (R)-4-(3-(but-2-ynamido)pyrrolidin-1-yl)-2-oxo-6-(4-phenoxyphenyl)-2,3- |
| | dihydro-1H-imidazo[4,5-c]pyridine-7-carboxamide |
| 10 | (S)-5-(3-(but-2-ynamido)piperidin-1-yl)-2,3-dioxo-7-(4-phenoxyphenyl)-1,2,3,4- |
| | tetrahydropyrido[3,4-b]pyrazine-8-carboxamide |
| 11 | (R)-5-(3-(but-2-ynamido)piperidin-1-yl)-2,3-dioxo-7-(4-phenoxyphenyl)-1,2,3,4- |
| | tetrahydropyrido[3,4-b]pyrazine-8-carboxamide |
| 12 | (S)-5-(3-acrylamidopiperidin-1-yl)-2,3-dioxo-7-(4-phenoxyphenyl)-1,2,3,4- |
| | tetrahydropyrido[3,4-b]pyrazine-8-carboxamide |
| 13 | (R)-5-(3-acrylamidopiperidin-1-yl)-2,3-dioxo-7-(4-phenoxyphenyl)-1,2,3,4- |
| | tetrahydropyrido[3,4-b]pyrazine-8-carboxamide |
| 14 | (S)-5-(3-(but-2-ynamido)pyrrolidin-1-yl)-2,3-dioxo-7-(4-phenoxyphenyl)-1,2,3,4- |
| | tetrahydropyrido[3,4-b]pyrazine-8-carboxamide |
| 15 | (R)-5-(3-(but-2-ynamido)pyrrolidin-1-yl)-2,3-dioxo-7-(4-phenoxyphenyl)-1,2,3,4- |
| | tetrahydropyrido[3,4-b]pyrazine-8-carboxamide |
| 16 | (S)-5-(3-acrylamidopyrrolidin-1-yl)-2,3-dioxo-7-(4-phenoxyphenyl)-1,2,3,4- |
| | tetrahydropyrido[3,4-b]pyrazine-8-carboxamide |
| 17 | (R)-5-(3-acrylamidopyrrolidin-1-yl)-2,3-dioxo-7-(4-phenoxyphenyl)-1,2,3,4- |
| | tetrahydropyrido[3,4-b]pyrazine-8-carboxamide |
| 18 | (S)-4-(3-(but-2-ynamido)piperidin-1-yl)-6-(4-phenoxyphenyl)-1H-imidazo[4,5- |
| | c]pyridine-7-carboxamide |
| 19 | (S)-4-(3-(but-2-ynamido)piperidin-1-yl)-2-methyl-6-(4-phenoxyphenyl)-1H- |
| | imidazo[4,5-c]pyridine-7-carboxamide |
| 20 | 4-(1-acryloylpiperidin-4-yl)-2-oxo-6-(4-phenoxyphenyl)-2,3-dihydro-1H- |
| | imidazo[4,5-c]pyridine-7-carboxamide |
| 21 | (S)-4-(1-acryloylpiperidin-3-yl)-2-oxo-6-(4-phenoxyphenyl)-2,3-dihydro-1H- |
| | imidazo[4,5-c]pyridine-7-carboxamide |
| 22 | (S)-4-(1-acryloylpiperidin-3-yl)-2-oxo-6-(4-phenoxyphenyl)-2,3-dihydro-1H- |
| | imidazo[4,5-c]pyridine-7-carboxamide |
| 23 | (S)-4-(3-(but-2-ynamido)piperidin-1-yl)-6-(4-phenoxyphenyl)-1H-pyrrolo[3,2- |
| | c]pyridine-7-carboxamide |
| 24 | (R)-4-(3-(but-2-ynamido)piperidin-1-yl)-6-(4-phenoxyphenyl)-1H-pyrrolo[3,2- |
| | c]pyridine-7-carboxamide |
| 25 | 4-(1-acryloylpiperidin-4-yl)-6-(4-phenoxyphenyl)-1H-pyrrolo[3,2-c]pyridine-7- |
| | carboxamide |
| 26 | (S)-4-(1-acryloylpiperidin-3-yl)-6-(4-phenoxyphenyl)-1H-pyrrolo[3,2-c]pyridine- |
| | 7-carboxamide |
| 27 | (R)-4-(1-acryloylpiperidin-3-yl)-6-(4-phenoxyphenyl)-1H-pyrrolo[3,2-c]pyridine- |
| | 7-carboxamide |
| 28 | (S)-5-(3-(but-2-ynamido)piperidin-1-yl)-2,3-dioxo-1,2,3,4-tetrahydropyrido[3,4- |
| | b]pyrazine-8-carboxamide |
| 29 | (R)-5-(3-(but-2-ynamido)piperidin-1-yl)-2,3-dioxo-1,2,3,4-tetrahydropyrido[3,4- |
| | b]pyrazine-8-carboxamide |
| 30 | (S)-5-(3-acrylamidopiperidin-1-yl)-2,3-dioxo-1,2,3,4-tetrahydropyrido[3,4- |
| | b]pyrazine-8-carboxamide |
| 31 | (R)-5-(3-acrylamidopiperidin-1-yl)-2,3-dioxo-1,2,3,4-tetrahydropyrido[3,4- |
| | b]pyrazine-8-carboxamide |
| 32 | (S)-5-(3-(but-2-ynamido)pyrrolidin-1-yl)-2,3-dioxo-1,2,3,4-tetrahydropyrido[3,4- |
| | b]pyrazine-8-carboxamide |
| 33 | (R)-5-(3-(but-2-ynamido)pyrrolidin-1-yl)-2,3-dioxo-1,2,3,4-tetrahydropyrido[3,4- |
| | b]pyrazine-8-carboxamide |
| 34 | (S)-5-(3-acrylamidopyrrolidin-1-yl)-2,3-dioxo-1,2,3,4-tetrahydropyrido[3,4- |
| | b]pyrazine-8-carboxamide |
| 35 | (R)-5-(3-acrylamidopyrrolidin-1-yl)-2,3-dioxo-1,2,3,4-tetrahydropyrido[3,4- |
| | b]pyrazine-8-carboxamide |
| 36 | (S)-4-(3-(but-2-ynamido)piperidin-1-yl)-1H-imidazo[4,5-c]pyridine-7- |
| | carboxamide |
| 37 | (S)-4-(3-(but-2-ynamido)piperidin-1-yl)-2-methyl-1H-imidazo[4,5-c]pyridine-7- |
| | carboxamide |
| 38 | (S)-4-(3-(but-2-ynamido)piperidin-1-yl)-2-oxo-2,3-dihydro-1H-imidazo[4,5- |
| | c]pyridine-7-carboxamide |
| 39 | (R)-4-(3-(but-2-ynamido)piperidin-1-yl)-2-oxo-2,3-dihydro-1H-imidazo[4,5- |
| | c]pyridine-7-carboxamide |
| 40 | 4-(1-acryloylpiperidin-4-yl)-2-oxo-2,3-dihydro-1H-imidazo[4,5-c]pyridine-7- |
| | carboxamide |
| 41 | (S)-4-(1-acryloylamino piperidine-3-yl)-2--methyl-1H-imidazo[4,5-c]pyridine-7- |
| | carboxamide |
| 42 | (R)-4-(1-acryloylamino piperidine-3-yl)-2--methyl-1H-imidazo[4,5-c]pyridine-7- |
| | carboxamide |

or prodrugs thereof, stable nuclide derivatives, pharmaceutically acceptable salts, isomers and mixtures and forms thereof.

6. A pharmaceutical composition, comprising compounds, pharmaceutically acceptable salts, isomers or prodrugs thereof according to claim 1, optionally, the pharmaceutical composition of the application further comprises one or more pharmaceutical excipients;
optionally, the pharmaceutical composition further comprises one or more second therapeutic agents;
preferably, the second therapeutic agent is selected from a chemotherapy drug, a targeted anticancer drug, or an immunotherapy drug; and
preferably, the second therapeutic agent is selected from rituximab, lenalidomide, fludarabine, Cyclophosphamide, doxorubicin, Vincristine and prednisone.

7. The compound, the pharmaceutically acceptable salt, the stereoisomer thereof and the use of the prodrugs thereof in preparing drugs according to claim 1, wherein the drugs are used for preventing and/or treating the diseases and/or symptoms related to the excessive activity of Bruton Tyrosine kinase in the subject;
preferably, the diseases and/or symptoms related to the excessive activity of Bruton Tyrosine kinase are selected from tumors (such as blood tumors or solid tumors), inflammation or autoimmune diseases;
preferably, the blood tumor is selected from lymphoma, myeloma, lymphocytic leukemia, and acute myeloid leukemia;
preferably, the solid tumor is selected from lung cancer, breast cancer, prostate cancer, stomach cancer, liver cancer, pancreatic cancer, ovarian cancer, and colon cancer;
preferably, the inflammation or autoimmune disease is selected from rheumatoid arthritis, lupus erythematosus, Lupus nephritis, Multiple sclerosis, Schogren's syndrome and underlying disease asthma;
preferably, a subject is a mammal; for example, bovine, equine, ovine, swine, canine, feline and, rodent; and a primate, for example, human;
preferably, the drug further comprises one or more second therapeutic agents;
preferably, the second therapeutic agent is a chemotherapy drug, a targeted anticancer drug, or an immunotherapy drug; and
preferably, the second therapeutic agent is selected from rituximab, lenalidomide, fludarabine, Cyclophosphamide, doxorubicin, Vincristine and prednisone.

8. The compound, the pharmaceutically acceptable salt, the stereoisomer thereof and the use of the prodrugs thereof in preparing drugs according to claim 1, wherein the preparation is used for reducing or inhibiting the activity of Bruton Tyrosine kinase in cells;
preferably, the preparation is administered to the body of the subject (such as a mammal:
including bovine, equine, ovine, swine, canine, feline and rodent, and a primate: including human) to reduce or inhibit the activity of Bruton Tyrosine kinase in the cells of the subject;
alternatively, the preparation is applied to cells in vitro (such as cell lines or cells from the subject) to reduce or inhibit the activity of Bruton Tyrosine kinase in cells in vitro;
preferably, the cells are selected from tumor cells (such as solid tumor cells, including lung cancer cells, breast cancer cells, prostate cancer cells, stomach cancer cells, liver cancer cells, pancreatic cancer cells, ovarian cancer cells, colon cancer cells);
preferably, the cells are selected from myeloid cells or lymphocytes; and
preferably, the cells are primary cells from the subject or the culture thereof, or established cell lines.

9. A kit, comprising the compound, the pharmaceutically acceptable salt, the stereoisomer or the prodrug thereof according to claim 1, and optionally the kit further comprises an instruction for use;
preferably, the kit is used for reducing or inhibiting the activity of Bruton Tyrosine kinase in cells;
preferably, the cells are selected from tumor cells (such as solid tumor cells, including lung cancer cells, breast cancer cells, prostate cancer cells, stomach cancer cells, liver cancer cells, pancreatic cancer cells, ovarian cancer cells, colon cancer cells);
preferably, the cells are selected from myeloid cells or lymphocytes; and
preferably, the cells are primary cells from the subject or the culture thereof, or established cell lines.
